(19) United States
(12) Patent Application Publication
(10) Pub. No.: US 2022/0047603 A1
(43) Pub. Date: Feb. 17, 2022

(54) SUBSTITUTED PYRROLOPYRIDINE-DERIVATIVES

(71) Applicants: BAYER AKTIENGESELLSCHAFT, Leverkusen (DE); DEUTSCHES KREBSFORSCHUNGSZENTRUM, Heidelberg (DE)

(72) Inventors: Jeffrey Stuart MOWAT, Antwerp (BE); Bernd BUCHMANN, Hohen Neuendorf (DE); Nuria AIGUABELLA FONT, Berlin (DE); Roland NEUHAUS, Berlin (DE); Gabriele LEDER, Berlin (DE); Rafael CARRETERO, Heidelberg (DE); Sandra BERNDT, Hohen Neuendorf (DE); Rienk OFFRINGA, Heidelberg (DE)

(73) Assignees: BAYER AKTIENGESELLSCHAFT, Leverkusen (DE); DEUTSCHES KREBSFORSCHUNGSZENTRUM, Heidelberg (DE)

(21) Appl. No.: 17/312,776

(22) PCT Filed: Dec. 4, 2019
(86) PCT No.: PCT/EP2019/083660

§ 371 Date: Jun. 10, 2021

(30) Foreign Application Priority Data
Dec. 11, 2018 (EP) 18211627.7

(51) Int. Cl. A61K 31/537; A61K 39/395; C07D 471/04; C07D 519/00; A61K 31/5355

(52) U.S. Cl. CPC A61K 31/537; A61K 39/3955; A61K 31/5355; C07D 519/00; C07D 471/04

(57) ABSTRACT

The present invention relates to protein-inhibitory substituted pyrrolopyridine derivatives of formula (I) in which A, X, R^{1a}, R^{1b}, R², R^{3a}, R^{3b}, R^{4a} and R^{4b} are as defined herein, to pharmaceutical compositions and combinations comprising the compounds according to the invention, and to the prophylactic and therapeutic use of the inventive compounds, respectively to the use of said compounds for manufacturing pharmaceutical compositions for the treatment or prophylaxis of diseases, in particular for neoplastic disorders, respectively cancer or conditions with dysregulated immune responses or other disorders associated with aberrant MAP4K1 signaling, as a sole agent or in combination with other active ingredients. The present invention further relates to the use, respectively to the use of said compounds for manufacturing pharmaceutical compositions for the treatment or prophylaxis of protein inhibitors in benign hyperplasias, atherosclerotic disorders, sepsis, autoimmune disorders, vascular disorders, viral infections, in neurodegenerative disorders, in inflammatory disorders, in atherosclerotic disorders and in male fertility control.

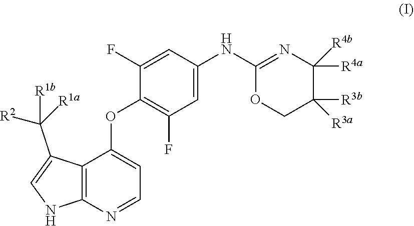

The present invention relates to protein-inhibitory substituted pyrrolopyridine derivatives, to pharmaceutical compositions and combinations comprising the compounds according to the invention, and to the prophylactic and therapeutic use of the inventive compounds, respectively to the use of said compounds for manufacturing pharmaceutical compositions for the treatment or prophylaxis of diseases, in particular for neoplastic disorders, respectively cancer or conditions with dysregulated immune responses or other disorders associated with aberrant MAP4K1 signaling, as a sole agent or in combination with other active ingredients.

The present invention further relates to the use, respectively to the use of said compounds for manufacturing pharmaceutical compositions for the treatment or prophylaxis of protein inhibitors in benign hyperplasias, atherosclerotic disorders, sepsis, autoimmune disorders, vascular disorders, viral infections, in neurodegenerative disorders, in inflammatory disorders, in atherosclerotic disorders and in male fertility control.

### BACKGROUND

Although cancer cell commonly can be recognize by the adaptive immune system, the response generated is evidently not capable of eliminating the tumor. A major reason for this is the presence of immunosuppressive mechanisms in the tumor microenvironment. In this respect, inhibitors of T-cell immune checkpoint such as CTLA-4, PD-1 or PD-L1 were recently shown to result in a remarkable clinical efficacy in subsets of cancer patients. Besides cell surface receptors that act as negative immune regulators, several mediators of intracellular signaling have been identified that also represent potential immunoevasive mechanisms utilized by the tumor. One of these is MAP4K1, also known as hematopoietic progenitor kinase 1 (HPK1). MAP4K1 (GeneID11184) is a serine/threonine kinase and member of the Germinal Center Kinase family. In the adult organism MAP4K1 expression is restricted to hematopoietic cell types. The MAP4K1 protein consist of a N-terminal kinase domain, followed by a proline-rich domain that can interact with adaptor molecules through SH2 and SH3 domains, and a C-terminal citron homology domain of which the exact function remains to be identified. Through its proline-rich domain, MAP4K1 is capable of binding to a diversity of adaptors in hematopoietic cells, including those involved in T-cell receptor (TCR), B-cell receptor (BCR) and cytokine signaling (Hu et al., Genes Dev. 1996 Sep. 15; 10(18):2251-64, 2.; Ling et al., J Biol Chem. 2001 Jun. 1; 276(22), Sauer et al., J Biol Chem. 2001 Nov. 30; 276(48):45207-16., Tsuji et al., J Exp Med. 2001 Aug. 20; 194(4):529-39, Boomer et al., J Cell Biochem. 2005 May 1; 95(1):34-44). The function of MAP4K1 has been studied in greatest detail in the context of TCR signaling. Upon TCR stimulation, MAP4K1 is phosphorylated on tyrosine 381 (Y-381; Y-379 in mouse) (Di Bartolo et al., J Exp Med. 2007 Mar. 19; 204(3):681-91). Consequently, MAP4K1 is recruited to the TCR-signaling complex where it induces dissociation of this complex through its serine/threonine kinase function. In particular MAP4K1 phosphorylates the SLP-76 adaptor protein at Serine-376, resulting in downregulation of AP-1 and Erk2 pathways. As, such, MAPK1 acts as a negative feedback on TCR-signaling (Liou et al., Immunity. 2000 April; 12(4):399-408; Lasserre et al., J Cell Biol. 2011 Nov. 28; 195(5):839-53.). Alternatively, MAP4K1 can be triggered to suppress T cell function by prostaglandin E2 (PGE2), and possibly also by transforming growth factor beta (TGF-beta), factors that are commonly found in the tumor microenvironment. Notably, MAP4K1 activation by these mediators involves protein kinase A (PKA)-dependent phosphorylation of Serine 171 (S-171; also in mouse) (Alzabin et al., Cancer Immunol Immunother. 2010 March; 59(3):419-29; Sawasdikosol et al., J Biol Chem. 2007 Nov. 30; 282(48):34693-9.).

Further important insights into the function of MAP4K1 in the regulation of T cell immunity stem from in vivo and in vitro experiments respectively with MAP4K1 deficient mice produced by two laboratories and with immune cells isolated from these mice (Shui et al., Nat Immunol. 2007 January; 8(1):84-91; Alzabin et al., Cancer Immunol Immunother. 2010 March; 59(3):419-29). MAP4K1-deficient mice show an apparent normal phenotype, are fertile and exhibit normal lymphocyte development. These animals are prone to develop T-cell dependent autoimmune reactivity as indicated by development of a more severe disease score in the EAE (experimental autoimmune encephalomyelitis) model of multiple sclerosis (Shui et al., Nat Immunol. 2007 Jan.; 8(1):84-91). In case of the second strain, a dysregulation of immune function was observed when, at the age of approximately 6 months, MAP4K1-deficient mice develop a spontaneous autoimmune phenotype (Alzabin et al., Cancer Immunol Immunother. 2010 March; 59(3):419-29). In vitro studies showed that MAP4K1−/− T-cells display hyper-responsiveness upon TCR-stimulation. These cells proliferate and secrete pro-inflammatory cytokines like IL-2 or IFNg to a significantly greater extent than their wild-type counterparts (Shui et al., Nat Immunol. 2007 January; 8(1):84-91). Furthermore, MAP4K1−/− T-cells are resistant to PGE2-mediated suppression of T cell proliferation, suppression of IL-2 production and induction of apoptosis (Alzabin et al Cancer Immunol Immunother. 2010 March; 59(3):419-29).

In the context of tumor immunology, in vivo experiments revealed that MAP4K1−/− mice are much more resistant to tumorigenesis by PGE2-producing Lewis lung carcinoma than wild type mice, which correlated with increased T-lymphocyte infiltration in the tumor areas. The crucial role of T-cells in tumor rejection was supported by experiments in which MAP4K1−/− T-cells adoptively transferred into T-cell-deficient mice were able to eradicate tumors more efficiently than wild-type T-cells (Alzabin et al., Cancer Immunol Immunother. 2010 March; 59(3):419-29). The important role of the kinase enzymatic activity was demonstrated by studies were only wild type MAP4K1, but not the MAP4K1 kinase-dead mutant, could mediate serine-phosphorylation of the TCR-signaling complex component SLP-76 and subsequent binding of SLP-76 to the negative regulator of TCR-signaling 14-3-3-t (Shui et al., Nat Immunol. 2007 January; 8(1):84-91). MAP4K1 also regulates the stimulation and activation of dendritic cells. MAP4K1 deficient Bone marrow derived cells (BMDC) express after maturation and stimulation higher level of costimulatory molecules and produce more proinflammatory cytokines. Also elimination of tumors was observed to be more efficient by MAP4K1 −/− BMDC compared to their wildtype counterparts (Alzabin et al., J Immunol. 2009 May 15; 182(10):6187-94).

### PRIOR ART

In WO 2018/215668, MAP4K1 (HPK1) inhibitors and methods for their use in diseases including hyperproliterative diseases, diseases of immune system dysfunction, inflammatory disorders, neurological diseases, and cardiovascular diseases are described. These compounds differ from the instant compounds in their chemical structure.

In WO 2018/049214, HPK1 modulators and methods for their use in cancer treatment are described. These compounds differ from the instant compounds in their chemical structure.

In WO 2018/049200, HPK1 modulators and methods for their use in cancer treatment are described. These compounds differ from the instant compounds in their chemical structure.

In WO 2018/049152, HPK1 modulators and methods for their use in cancer treatment are described. These compounds differ from the instant compounds in their chemical structure.

In WO 2018/049119, HPK1 modulators and methods for their use in cancer treatment are described. These compounds differ from the instant compounds in their chemical structure.

In WO 2018/102366, HPK1 inhibitors and methods for their use in the treatment of cancer are described. These compounds differ from the instant compounds in their chemical structure.

In WO 2018/183956, HPK1 inhibitors and use ot such compounds in treating HPK1-dependent disorders and enhancing immune response are described. These compounds differ from the instant compounds in their chemical structure.

In WO 2018/183964, HPK1 inhibitors and use ot such compounds in treating HPK1-dependent disorders and enhancing immune response are described. These compounds differ from the instant compounds in their chemical structure.

In WO 2018/167147, HPK1 inhibitors and use ot such compounds in treating HPK1-dependent disorders and enhancing immune response are described. These compounds differ from the instant compounds in their chemical structure. In WO 2016/205942

HPK1, respectively inhibitors and methods of their use in cancer treatment are described. Especially, the application concerns thieno-pyridinones that can be used in anti-cancer therapy. These compounds differ from the instant compounds in their chemical structure.

In WO 2016/195776 inhibitors and methods for leukemia, cancer and diabetes treatment dependent on inhibition the interaction of menin with of MLL1, MLL2 and MLL-fusion oncoproteins are described. These compounds differ from the instant compounds in their chemical structure.

In WO 2006/014325 C-MET modulators and their use in cancer treatment are described. These compounds differ from the instant compounds in their chemical structure.

In WO 2005/058891 Rho kinase inhibitors and their use in cardiovascular and cancer treatment are described. These compounds differ from the instant compounds in their chemical structure.

In WO 2015/089479 several inhibitors are described that show inhibition of several kinases (e.g., BTK, HCK, TAK1 and HPK1). These compounds differ from the instant compounds in their chemical structure.

In WO2016/004272 BTK inhibitors and methods of their use in cancer treatment are described. No specific example is disclosed which falls in the group of compounds as defined according to the present invention.

In WO 2011/090738 Type II RAF kinase inhibitors and their use in various diseases are described. No specific example is disclosed which falls in the group of compounds as defined according to the present invention.

In CN102086211 and WO2006116713 protein kinase inhibitors and their use in prophylaxis and treatment of diseases including cancer are described. No specific example is disclosed which falls in the group of compounds as defined according to the present invention.

In WO 2010/045095 protein tyrosin kinase modulators and their use in the treatment of hyperproliferative disorders are described. No specific example is disclosed which falls in the group of compounds as defined according to the present invention.

In WO 2008/089307 compounds and methods of their use in the treatment of pain, inflammation and cancer are described. No specific example is disclosed which falls in the group of compounds as defined according to the present invention.

In WO 2006/114180 kinase inhibitors for treating diseases, particularly tumors are described. No specific example is disclosed which falls in the group of compounds as defined according to the present invention.

In WO 2006/014325 c-Met modulators and their methods of use to treat kinase-dependent diseases and conditions are described. No specific example is disclosed which falls in the group of compounds as defined according to the present invention.

In US 2003/0055049 compounds for treating disorders with abnormal cell growth in mammals are described. No specific example is disclosed which falls in the group of compounds as defined according to the present invention.

In WO 2001/23389 antagonists of NPY receptors compositions and methods of the treatment of physiological disorders associated with an excess of neuropeptide Y are described. No specific example is disclosed which falls in the group of compounds as defined according to the present invention.

It would therefore be desirable to provide novel compounds having prophylactic and therapeutic properties.

Accordingly, it is an object of the present invention to provide compounds and pharmaceutical compositions comprising these compounds used for prophylactic and therapeutic applications for hyperproliferative disorders, in particular for cancer, respectively tumour disorders, and conditions with dysregulated immune responses, as a sole agent or in combination with other active ingredients.

A further object of the present invention is to provide compounds and pharmaceutical compositions comprising these compounds for manufacturing pharmaceutical compositions for the treatment or prophylaxis of protein inhibitors in benign hyperplasias, atherosclerotic disorders, sepsis, autoimmune disorders, vascular disorders, viral infections, in neurodegenerative disorders, in inflammatory disorders, in atherosclerotic disorders and in male fertility control.

A further object of the present invention is to provide compounds that inhibit the MAP4K1 protein and inhibit the growth of cancer cells.

A further object of the present invention is to provide compounds that inhibit selectively the MAP4K1 protein and inhibit the growth of cancer cells, especially are selective against the Rock II kinase i.e. inhibit the Rock II kinase at least 30 times less than the MAP4K1 protein.

Preferably, the Rock II kinase shall be inhibited 50 times, more preferably 100 times less than the MAP4K1 protein.

Inhibition of the Rock II kinase is associated with potential tolerability and safety issues. For example, Rock II kinase is described to play several roles in diverse cellular processes such as cytoskeletal re-organization, and gene expression regulation, the latter of which functions in cell migration, contraction and cell proliferation. Additionally, Rock II inhibition can potentially lead to effects on haemodynamic functions i.e. effects on blood pressure and heart rate. Moreover, Rock II kinase inhibition carries the risk to interfere with cytokinesis during cell division and consequently perturb proper daughter cell formation. Therefore there is a high need for MAP4K1 inhibitors that are selective to the Rock-II kinase.

Surprisingly, the compounds according to the invention inhibit the MAP4K1 protein and inhibit the growth of cancer cells. Accordingly, they provide novel structures for the therapy of human and animal disorders, in particular of cancers.

The present invention relates to compounds of formula (I)

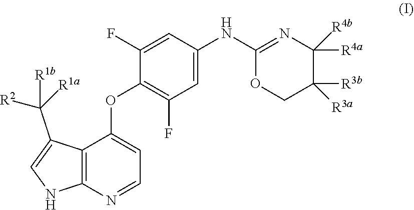

in which
R^{1a} and R^{1b} together represent a C₃-C₆-cycloalkyl ring,
R² represents a C₁-C₃-haloalkyl
R^{3a} and R^{3b} represent independently from each other hydrogen or a C₁-C₃-alkyl
R^{4a} and R^{4b} represent independently from each other hydrogen or a C₁-C₃-alkyl or
R^{3a} and R^{3b} together represent a monocyclic 3 to 6-membered cycloalkyl or 4 to 6-membered heterocycloalkyl, wherein said cycloalkyl or heterocycloalkyl is optionally substituted by fluorine, chlorine, methyl, methoxy, hydroxy, cyano,

and their polymorphs, enantiomers, diastereomers, racemates, tautomers, solvates, physiologically acceptable salts and solvates of these salts.

The compounds of formula (I) are particularly suitable for a large number of prophylactic and therapeutic applications, in particular for hyperproliferative disorders, for tumour disorders and as proteine inhibitors and further for viral infections, for neurodegenerative disorders, for inflammatory disorders, for atherosclerotic disorders and for male fertility control.

Further, it covers their use in combination with other anti cancer medications such as immunotherapeutics, targeted anti cancer agents, radiation or chemotherapy.

### Definitions

The term “substituted” means that one or more hydrogen atoms on the designated atom or group are replaced with a selection from the indicated group, provided that the designated atom's normal valency under the existing circumstances is not exceeded. Combinations of substituents and/or variables are permissible.

The term “optionally substituted” means that the number of substituents can be equal to or different from zero. Unless otherwise indicated, it is possible that optionally substituted groups are substituted with as many optional substituents as can be accommodated by replacing a hydrogen atom with a non-hydrogen substituent on any available carbon or nitrogen or . . . atom. Commonly, it is possible for the number of optional substituents, when present, to be 1, 2, 3, 4 or 5, in particular 1, 2 or 3.

As used herein, the term “one or more”, e.g. in the definition of the substituents of the compounds of general formula (I) of the present invention, means “1, 2, 3, 4 or 5, particularly 1, 2, 3 or 4, more particularly 1, 2 or 3, even more particularly 1 or 2”.

When groups in the compounds according to the invention are substituted, it is possible for said groups to be mono-substituted or poly-substituted with substituent(s), unless otherwise specified. Within the scope of the present invention, the meanings of all groups which occur repeatedly are independent from one another. It is possible that groups in the compounds according to the invention are substituted with one, two or three identical or different substituents, particularly with one substituent.

As used herein, an oxo substituent represents an oxygen atom, which is bound to a carbon atom or to a sulfur atom via a double bond.

The term “ring substituent” means a substituent attached to an aromatic or nonaromatic ring which replaces an available hydrogen atom on the ring.

The term “comprising” when used in the specification includes “consisting of”.

If within the present text any item is referred to as “as mentioned herein”, it means that it may be mentioned anywhere in the present text.

The terms as mentioned in the present text have the following meanings:

The term “halogen atom” means a fluorine, chlorine, bromine or iodine atom, particularly a fluorine, chlorine or bromine atom.

The term “C₁-C₃-alkyl” means a linear or branched, saturated, monovalent hydrocarbon group having 1, 2 or 3 carbon atoms (“C₁-C₃-alkyl”), e.g. a methyl, ethyl, n-propyl or isopropyl group.

The term “C₁-C₃-haloalkyl” means a linear or branched, saturated, monovalent hydrocarbon group in which the term “C₁-C₃-alkyl” is as defined supra, and in which one or more of the hydrogen atoms are replaced, identically or differently, with a halogen atom. Particularly, said halogen atom is a fluorine atom. Said C₁-C₃-haloalkyl group is, for example, fluoromethyl, difluoromethyl, trifluoromethyl, 2-fluoroethyl, 2,2-difluoroethyl, 2,2,2-trifluoroethyl, pentafluoroethyl, 3,3,3-trifluoropropyl or 1,3-difluoropropan-2-yl.

Preference is given to perfluorinated alkyl radicals which are named as “perfluoro-C₁-Cₓ-alkyl-” wherein x is the maximum number of carbon atoms such as trifluoromethyl.

The term “C₃-C₆-cycloalkyl” is equivalent to “3- to 6-membered cycloalkyl” and means a saturated, monovalent, monocyclic hydrocarbon ring which contains 3, 4, 5 or 6 carbon atoms (“C₃-C₆-cycloalkyl”). Said C₃-C₆-cycloalkyl group or “3- to 6-membered cycloalkyl” is a cyclopropyl, cyclobutyl, cyclopentyl or cyclohexyl group.

A C₃-C₅-cycloalkyl group or “3- to 5-membered cycloalkyl” is a cyclopropyl, cyclobutyl, or cyclopentyl group.

The terms “4- to 6-membered heterocycloalkyl” mean a monocyclic, saturated or unsaturated heterocycle with 4, 5, or 6 ring atoms in total, which contains one or two identical or different ring heteroatoms from the series N, O and S, it being possible for said heterocycloalkyl group to be attached to the rest of the molecule via any one of the carbon atoms or, if present, a nitrogen atom. A carbon atom may be substituted with an oxo group or or the sulphur atom with one or two oxo groups to form a —C═O, —S(═O)— or —S(═O)₂— group in the ring. Preferred heteroatom is O.

Said heterocycloalkyl group, without being limited thereto, can be a 4-membered ring, such as azetidinyl, oxetanyl or thietanyl, for example; or a 5-membered ring, such as tetrahydrofuranyl, 1,3-dioxolanyl, thiolanyl, pyrrolidinyl, imidazolidinyl, pyrazolidinyl, 1,1-dioxidothiolanyl, 1,2-oxazolidinyl, 1,3-oxazolidinyl or 1,3-thiazolidinyl, for example; or a 6-membered ring, such as tetrahydropyranyl, tetrahydrothiopyranyl, piperidinyl, morpholinyl, dithianyl, thiomorpholinyl, piperazinyl, 1,3-dioxanyl, 1,4-dioxanyl or 1,2-oxazinanyl.

As used herein, the term “leaving group” means an atom or a group of atoms that is displaced in a chemical reaction as stable species taking with it the bonding electrons. In particular, such a leaving group is selected from the group comprising: halide, in particular fluoride, chloride, bromide or iodide, (methylsulfonyl)oxy, [(trifluoromethyl)sulfonyl]oxy, [(nonafluorobutyl)sulfonyl]oxy, (phenylsulfonyl)oxy, [(4-methylphenyl)sulfonyl]oxy, [(4-bromophenyl)sulfonyl]oxy, [(4-nitrophenyl)sulfonyl]oxy, [(2-nitrophenyl)sulfonyl]oxy, [(4-isopropylphenyl)sulfonyl]oxy, [(2,4,6-triisopropylphenyl)sulfonyl]oxy, [(2,4,6-trimethylphenyl)sulfonyl]oxy, [(4-tert-butylphenyl)sulfonyl]oxy and [(4-methoxyphenyl)sulfonyl]oxy.

Where the plural form of the word compounds, salts, polymorphs, hydrates, solvates and the like, is used herein, this is taken to mean also a single compound, salt, polymorph, isomer, hydrate, solvate or the like.

By “stable compound” or “stable structure” is meant a compound that is sufficiently robust to survive isolation to a useful degree of purity from a reaction mixture, and formulation into an efficacious therapeutic agent.

The compounds of the present invention optionally contain one or more asymmetric centres, depending upon the location and nature of the various substituents desired. It is possible that one or more asymmetric carbon atoms are present in the (R) or (S) configuration, which can result in racemic mixtures in the case of a single asymmetric centre, and in diastereomeric mixtures in the case of multiple asymmetric centres. In certain instances, it is possible that asymmetry also be present due to restricted rotation about a given bond, for example, the central bond adjoining two substituted aromatic rings of the specified compounds.

Preferred compounds are those which produce the more desirable biological activity. Separated, pure or partially purified isomers and stereoisomers or racemic or diastereomeric mixtures of the compounds of the present invention are also included within the scope of the present invention. The purification and the separation of such materials can be accomplished by standard techniques known in the art.

Preferred isomers are those which produce the more desirable biological activity. These separated, pure or partially purified isomers or racemic mixtures of the compounds of this invention are also included within the scope of the present invention. The purification and the separation of such materials can be accomplished by standard techniques known in the art.

The optical isomers can be obtained by resolution of the racemic mixtures according to conventional processes, for example, by the formation of diastereoisomeric salts using an optically active acid or base or formation of covalent diastereomers. Examples of appropriate acids are tartaric, diacetyltartaric, ditoluoyltartaric and camphorsulfonic acid. Mixtures of diastereoisomers can be separated into their individual diastereomers on the basis of their physical and/or chemical differences by methods known in the art, for example, by chromatography or fractional crystallisation. The optically active bases or acids are then liberated from the separated diastereomeric salts. A different process for separation of optical isomers involves the use of chiral chromatography (e.g., HPLC columns using a chiral phase), with or without conventional derivatisation, optimally chosen to maximise the separation of the enantiomers. Suitable HPLC columns using a chiral phase are commercially available, such as those manufactured by Daicel, e.g., Chiracel OD and Chiracel OJ, for example, among many others, which are all routinely selectable. Enzymatic separations, with or without derivatisation, are also useful. The optically active compounds of the present invention can likewise be obtained by chiral syntheses utilizing optically active starting materials.

In order to distinguish different types of isomers from each other reference is made to IUPAC Rules Section E (Pure Appl Chem 45, 11-30, 1976).

The present invention includes all possible stereoisomers of the compounds of the present invention as single stereoisomers, or as any mixture of said stereoisomers, e.g. (R)- or (S)-isomers, in any ratio. Isolation of a single stereoisomer, e.g. a single enantiomer or a single diastereomer, of a compound of the present invention is achieved by any suitable state of the art method, such as chromatography, especially chiral chromatography, for example.

Further, the compounds of the present invention can exist as N-oxides, which are defined in that at least one nitrogen of the compounds of the present invention is oxidised. The present invention includes all such possible N-oxides.

The present invention also covers useful forms of the compounds of the present invention, such as metabolites, hydrates, solvates, prodrugs, salts, in particular pharmaceutically acceptable salts, and/or co-precipitates.

The compounds of the present invention can exist as a hydrate, or as a solvate, wherein the compounds of the present invention contain polar solvents, in particular water, methanol or ethanol for example, as structural element of the crystal lattice of the compounds. It is possible for the amount of polar solvents, in particular water, to exist in a stoichiometric or non-stoichiometric ratio. In the case of stoichiometric solvates, e.g. a hydrate, hemi-, (semi-), mono-, sesqui-, di-, tri-, tetra-, penta-etc. solvates or hydrates, respectively, are possible. The present invention includes all such hydrates or solvates.

Further, it is possible for the compounds of the present invention to exist in free form, e.g. as a free base, or as a free acid, or as a zwitterion, or to exist in the form of a salt. Said salt may be any salt, either an organic or inorganic addition salt, particularly any pharmaceutically acceptable organic or inorganic addition salt, which is customarily used in pharmacy, or which is used, for example, for isolating or purifying the compounds of the present invention.

The term “pharmaceutically acceptable salt” refers to an inorganic or organic acid addition salt of a compound of the present invention. For example, see S. M. Berge, et al. “Pharmaceutical Salts,” J. Pharm. Sci. 1977, 66, 1-19.

A suitable pharmaceutically acceptable salt of the compounds of the present invention may be, for example, an acid-addition salt of a compound of the present invention bearing a nitrogen atom, in a chain or in a ring, for example, which is sufficiently basic, such as an acid-addition salt with an inorganic acid, or “mineral acid”, such as hydrochloric, hydrobromic, hydroiodic, sulfuric, sulfamic, bisulfuric, phosphoric, or nitric acid, for example, or with an organic acid, such as formic, acetic, acetoacetic, pyruvic, trifluoroacetic, propionic, butyric, hexanoic, heptanoic, undecanoic, lauric, benzoic, salicylic, 2-(4-hydroxybenzoyl)-benzoic, camphoric, cinnamic, cyclopentanepropionic, digluconic, 3-hydroxy-2-naphthoic, nicotinic, pamoic, pectinic, 3-phenylpropionic, pivalic, 2-hydroxyethanesulfonic, itaconic, trifluoromethanesulfonic, dodecylsulfuric, ethanesulfonic, benzenesulfonic, para-toluenesulfonic, methanesulfonic, 2-naphthalenesulfonic, naphthalinedisulfonic, camphorsulfonic acid, citric, tartaric, stearic, lactic, oxalic, malonic, succinic, malic, adipic, alginic, maleic, fumaric, D-gluconic, mandelic, ascorbic, glucoheptanoic, glycerophosphoric, aspartic, sulfosalicylic, or thiocyanic acid, for example.

Further, another suitably pharmaceutically acceptable salt of a compound of the present invention which is sufficiently acidic, is an alkali metal salt, for example a sodium or potassium salt, an alkaline earth metal salt, for example a calcium, magnesium or strontium salt, or an aluminium or a zinc salt, or an ammonium salt derived from ammonia or from an organic primary, secondary or tertiary amine having 1 to 20 carbon atoms, such as ethylamine, diethylamine, triethylamine, ethyldiisopropylamine, monoethanolamine, diethanolamine, triethanolamine, dicyclohexylamine, dimethylaminoethanol, diethylaminoethanol, tris(hydroxymethyl)aminomethane, procaine, dibenzylamine, N-methylmorpholine, arginine, lysine, 1,2-ethylenediamine, N-methylpiperidine, N-methyl-glucamine, N,N-dimethyl-glucamine, N-ethyl-glucamine, 1,6-hexanediamine, glucosamine, sarcosine, serinol, 2-amino-1,3-propanediol, 3-amino-1,2-propanediol, 4-amino-1,2,3-butanetriol, or a salt with a quarternary ammonium ion having 1 to 20 carbon atoms, such as tetramethylammonium, tetraethylammonium, tetra(n-propyl)ammonium, tetra(n-butyl)ammonium, N-benzyl-N,N,N-trimethylammonium, choline or benzalkonium.

Those skilled in the art will further recognise that it is possible for acid addition salts of the claimed compounds to be prepared by reaction of the compounds with the appropriate inorganic or organic acid via any of a number of known methods. Alternatively, alkali and alkaline earth metal salts of acidic compounds of the present invention are prepared by reacting the compounds of the present invention with the appropriate base via a variety of known methods.

The present invention includes all possible salts of the compounds of the present invention as single salts, or as any mixture of said salts, in any ratio.

In the present text, in particular in the Experimental Section, for the synthesis of intermediates and of examples of the present invention, when a compound is mentioned as a salt form with the corresponding base or acid, the exact stoichiometric composition of said salt form, as obtained by the respective preparation and/or purification process, is, in most cases, unknown.

Unless specified otherwise, suffixes to chemical names or structural formulae relating to salts, such as “hydrochloride”, “trifluoroacetate”, “sodium salt”, or “x HCl”, “x CF₃COOH”, “x Na⁺”, for example, mean a salt form, the stoichiometry of which salt form not being specified.

This applies analogously to cases in which synthesis intermediates or example compounds or salts thereof have been obtained, by the preparation and/or purification processes described, as solvates, such as hydrates, with (if defined) unknown stoichiometric composition.

Furthermore, the present invention includes all possible crystalline forms, or polymorphs, of the compounds of the present invention, either as single polymorph, or as a mixture of more than one polymorph, in any ratio.

Moreover, the present invention also includes prodrugs of the compounds according to the invention. The term “prodrugs” here designates compounds which themselves can be biologically active or inactive, but are converted (for example metabolically or hydrolytically) into compounds according to the invention during their residence time in the body.

The invention further includes all possible crystallized and polymorphic forms of the inventive compounds, whereby the polymorphs are existing either as a single polymorph form or are existing as a mixture of several polymorphs in all concentrations.

The invention further includes all possible cyclodextrin clathrates, i.e. alpha-, beta-, or gamma-cyclodextrins, hydroxypropyl-beta-cyclodextrins, methylbetacyclodextrins.

Further, it is possible for the compounds of the present invention to exist as tautomers or mixtures of tautomers, namely having instead of or in addition to the substructures defined for A of compounds according to formula (I) following moieties

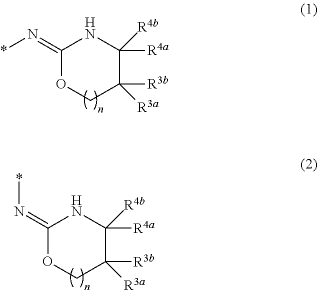

* connected to the benzene ring
The present invention includes all possible tautomers of the compounds of the present invention as single tautomers, or as any mixture of said tautomers, in any ratio

Of selected interest are those compounds of formula (I),

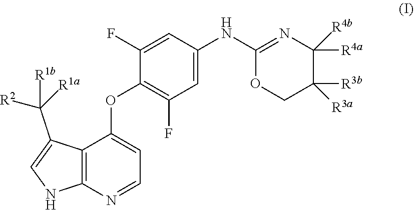

in which
R^{1a} and R^{1b} together represent a C₃-C₅-cycloalkyl ring,
R² represents a halomethyl or haloethyl group
R^{3a} and R^{3b} represent hydrogen or a methyl group
R^{4a} and R^{4b} represent hydrogen or a methyl group or
R^{3a} and R^{3b} together represent a monocyclic 3 to 5-membered cycloalkyl or 5 or 6-membered heterocycloalkyl, wherein said cycloalkyl or heterocycloalkyl is optionally substituted by fluorine, chlorine, methyl, methoxy, hydroxy, cyano,

and their polymorphs, enantiomers, diastereomers, racemates, tautomers, solvates, physiologically acceptable salts and solvates of these salts.

Of selected interest are those compounds of formula (I), in which

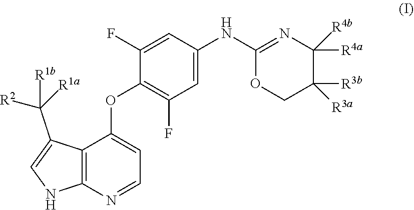

in which
R^{1a} and R^{1b} together represent a cyclopropyl or cyclobutyl ring,
R² represents a trifluoromethyl
R^{3a} and R^{3b} represent a methyl group or
R^{3a} and R^{3b} together represent a cyclopropyl or cyclobutyl ring or a 5 or 6-membered heterocycloalkyl ring having an oxygen atom as heteroatom
R^{4a} and R^{4b} represent hydrogen

and their polymorphs, enantiomers, diastereomers, racemates, tautomers, solvates, physiologically acceptable salts and solvates of these salts.

Of selected interest are those compounds of formula (I), in which

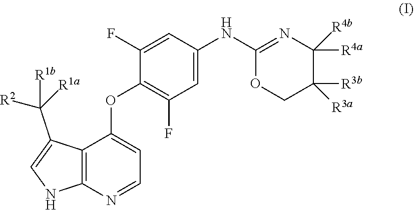

in which
R^{1a} and R^{1b} together represent a cyclobutyl ring,
R² represents a trifluoromethyl
R^{3a} and R^{3b} represent both a methyl group or
R^{3a} and R^{3b} together represent a cyclopropyl or cyclobutyl ring or a 6-membered heterocycloalkyl ring having an oxygen atom as heteroatom
R^{4a} and R^{4b} represent hydrogen

and their polymorphs, enantiomers, diastereomers, racemates, tautomers, solvates, physiologically acceptable salts and solvates of these salts.

Compounds of most interest are those as follows:
N-[3,5-difluoro-4-({3-[1-(trifluoromethyl)cyclobutyl]-1H-pyrrolo[2,3-b]pyridin-4-yl}oxy)phenyl]-6-oxa-8-azaspiro[3.5]non-7-en-7-amine
N-[3,5-difluoro-4-({3-[1-(trifluoromethyl)cyclobutyl]-1H-pyrrolo[2,3-b]pyridin-4-yl}oxy)phenyl]-2,9-dioxa-4-azaspiro[5.5]undec-3-en-3-amine
N-[3,5-difluoro-4-({3-[1-(trifluoromethyl)cyclobutyl]-1H-pyrrolo[2,3-b]pyridin-4-yl}oxy)phenyl]-5-oxa-7-azaspiro[2.5]oct-6-en-6-amine
N-[3,5-difluoro-4-({3-[1-(trifluoromethyl)cyclobutyl]-1H-pyrrolo[2,3-b]pyridin-4-yl}oxy)phenyl]-5,5-dimethyl-5,6-dihydro-4H-1,3-oxazin-2-amine
(+/−)-N-[3,5-difluoro-4-({3-[1-(trifluoromethyl)cyclopropyl]-1H-pyrrolo[2,3-b]pyridin-4-yl}oxy)phenyl]-2,7-dioxa-9-azaspiro[4.5]dec-8-en-8-amine
(5R)—N-[3,5-difluoro-4-({3-[1-(trifluoromethyl)cyclopropyl]-1H-pyrrolo[2,3-b]pyridin-4-yl}oxy)phenyl]-2,7-dioxa-9-azaspiro[4.5]dec-8-en-8-amine
(5S)—N-[3,5-difluoro-4-({3-[1-(trifluoromethyl)cyclopropyl]-1H-pyrrolo[2,3-b]pyridin-4-yl}oxy)phenyl]-2,7-dioxa-9-azaspiro[4.5]dec-8-en-8-amine
N-[3,5-difluoro-4-({3-[1-(trifluoromethyl)cyclopropyl]-1H-pyrrolo[2,3-b]pyridin-4-yl}oxy)phenyl]-2,7-dioxa-9-azaspiro[4.5]dec-8-en-8-amine (single enantiomer 1)
N-[3,5-difluoro-4-({3-[1-(trifluoromethyl)cyclopropyl]-1H-pyrrolo[2,3-b]pyridin-4-yl}oxy)phenyl]-2,7-dioxa-9-azaspiro[4.5]dec-8-en-8-amine (single enantiomer 2)
N-[3,5-difluoro-4-({3-[1-(trifluoromethyl)cyclopropyl]-1H-pyrrolo[2,3-b]pyridin-4-yl}oxy)phenyl]-2,6-dioxa-8-azaspiro[3.5]non-7-en-7-amine
N-[3,5-difluoro-4-({3-[1-(trifluoromethyl)cyclopropyl]-1H-pyrrolo[2,3-b]pyridin-4-yl}oxy)phenyl]-5,5-dimethyl-5,6-dihydro-4H-1,3-oxazin-2-amine
N-[3,5-difluoro-4-({3-[1-(trifluoromethyl)cyclopropyl]-1H-pyrrolo[2,3-b]pyridin-4-yl}oxy)phenyl]-5-oxa-7-azaspiro[2.5]oct-6-en-6-amine
N-[3,5-difluoro-4-({3-[1-(trifluoromethyl)cyclopropyl]-1H-pyrrolo[2,3-b]pyridin-4-yl}oxy)phenyl]-2,9-dioxa-4-azaspiro[5.5]undec-3-en-3-amine
N-[3,5-difluoro-4-({3-[1-(trifluoromethyl)cyclopropyl]-1H-pyrrolo[2,3-b]pyridin-4-yl}oxy)phenyl]-6-oxa-8-azaspiro[3.5]non-7-en-7-amine
(+/−)-N-[3,5-difluoro-4-({3-[1-(trifluoromethyl)cyclobutyl]-1H-pyrrolo[2,3-b]pyridin-4-yl}oxy)phenyl]-2,7-dioxa-9-azaspiro[4.5]dec-8-en-8-amine
N-[3,5-difluoro-4-({3-[1-(trifluoromethyl)cyclobutyl]-1H-pyrrolo[2,3-b]pyridin-4-yl}oxy)phenyl]-2,6-dioxa-8-azaspiro[3.5]non-7-en-7-amine

and their polymorphs, enantiomers, diastereomers, racemates, tautomers, solvates, physiologically acceptable salts and solvates of these salts.

In accordance with a further embodiment, the present invention covers compounds of general formula (I) in which

R^{1a} and R^{1b} together represent a C₃-C₆-cycloalkyl ring.

In accordance with a further embodiment, the present invention covers compounds of general formula (I) in which

R^{1a} and R^{1b} together represent a C₃-C₅-cycloalkyl ring.

In accordance with a further embodiment, the present invention covers compounds of general formula (I) in which

R^{1a} and R^{1b} together represent a cyclopropyl or cyclobutyl ring.

In accordance with a further embodiment, the present invention covers compounds of general formula (I) in which

R^{1a} and R^{1b} together represent a cyclobutyl ring.

In accordance with a further embodiment, the present invention covers compounds of general formula (I) in which

R² represents a C₁-C₃-haloalky.

In accordance with a further embodiment, the present invention covers compounds of general formula (I) in which

R² represents a halomethyl or haloethyl group.

In accordance with a further embodiment, the present invention covers compounds of general formula (I) in which

R² represents a trifluoromethyl.

In accordance with a further embodiment, the present invention covers compounds of general formula (I) in which

R^{3a} and R^{3b} represent independently from each other hydrogen or a C₁-C₃-alkyl.

In accordance with a further embodiment, the present invention covers compounds of general formula (I) in which

R^{3a} and R^{3b} together represent a monocyclic 3 to 6-membered cycloalkyl or 4 to 6-membered heterocycloalkyl, wherein said cycloalkyl or heterocycloalkyl is optionally substituted by fluorine, chlorine, methyl, methoxy, hydroxy, cyano.

In accordance with a further embodiment, the present invention covers compounds of general formula (I) in which

R^{3a} and R^{3b} represent hydrogen or a methyl group.

In accordance with a further embodiment, the present invention covers compounds of general formula (I) in which
R^{3a} and R^{3b} together represent a monocyclic 3 to 5-membered cycloalkyl or 5 or 6-membered heterocycloalkyl, wherein said cycloalkyl or heterocycloalkyl is optionally substituted by fluorine, chlorine, methyl, methoxy, hydroxy, cyano.

In accordance with a further embodiment, the present invention covers compounds of general formula (I) in which

R^{3a} and R^{3b} represent both a methyl group.

In accordance with a further embodiment, the present invention covers compounds of general formula (I) in which
R^{3a} and R^{3b} together represent a cyclopropyl or cyclobutyl ring or a 5 or 6-membered heterocycloalkyl ring having an oxygen atom as heteroatom.

In accordance with a further embodiment, the present invention covers compounds of general formula (I) in which
R^{3a} and R^{3b} together represent a cyclopropyl or cyclobutyl ring or a 6-membered heterocycloalkyl ring having an oxygen atom as heteroatom.

In accordance with a further embodiment, the present invention covers compounds of general formula (I) in which

R^{4a} and R^{4b} represent independently from each other hydrogen or a C₁-C₃-alkyl. In accordance with a further embodiment, the present invention covers compounds of general formula (I) in which

R^{4a} and R^{4b} represent hydrogen or a methyl group.

In accordance with a further embodiment, the present invention covers compounds of general formula (I) in which

R^{4a} and R^{4b} represent hydrogen.

The compounds of general formula (I) of the present invention can be converted to any salt, preferably pharmaceutically acceptable salts, as described herein, by any method which is known to the person skilled in the art. Similarly, any salt of a compound of general formula (I) of the present invention can be converted into the free compound, by any method which is known to the person skilled in the art.

Compounds of general formula (I) of the present invention demonstrate a valuable pharmacological spectrum of action, which could not have been predicted. Compounds of the present invention have surprisingly been found to effectively inhibit MAP4K1 and it is possible therefore that said compounds be used for the treatment or prophylaxis of diseases, preferably cancer or conditions with dysregulated immune responses or other disorders associated with aberrant MAP4K1 signaling, in humans and animals.

Disorders and conditions particularly suitable for treatment with an MAP4K1 inhibitor of the present invention are liquid and solid tumours, such as cancers of the breast, respiratory tract, brain, reproductive organs, digestive tract, urinary tract, eye, liver, skin, head and neck, thyroid, parathyroid and their distant metastases. Those disorders also include lymphomas, sarcomas, and leukaemias.

Examples of breast cancers include, but are not limited to, triple negative breast cancer, invasive ductal carcinoma, invasive lobular carcinoma, ductal carcinoma in situ, and lobular carcinoma in situ.

Examples of cancers of the respiratory tract include, but are not limited to, small-cell and non-small-cell lung carcinoma, as well as bronchial adenoma and pleuropulmonary blastoma.

Examples of brain cancers include, but are not limited to, brain stem and hypothalmic glioma, cerebellar and cerebral astrocytoma, glioblastoma, medulloblastoma, ependymoma, as well as neuroectodermal and pineal tumour.

Tumours of the male reproductive organs include, but are not limited to, prostate and testicular cancer.

Tumours of the female reproductive organs include, but are not limited to, endometrial, cervical, ovarian, vaginal, and vulvar cancer, as well as sarcoma of the uterus.

Examples of ovarian cancer include, but are not limited to serous tumour, endometrioid tumour, mucinous cystadenocarcinoma, granulosa cell tumour, Sertoli-Leydig cell tumour and arrhenoblastoma.

Examples of cervical cancer include, but are not limited to squamous cell carcinoma, adenocarcinoma, adenosquamous carcinoma, small cell carcinoma, neuroendocrine tumour, glassy cell carcinoma and villoglandular adenocarcinoma.

Tumours of the digestive tract include, but are not limited to, anal, colon, colorectal, esophageal, gallbladder, gastric, pancreatic, rectal, small-intestine, and salivary gland cancers.

Examples of esophageal cancer include, but are not limited to esophageal cell carcinomas and adenocarcinomas, as well as squamous cell carcinomas, leiomyosarcoma, malignant melanoma, rhabdomyosarcoma and lymphoma.

Examples of gastric cancer include, but are not limited to intestinal type and diffuse type gastric adenocarcinoma.

Examples of pancreatic cancer include, but are not limited to ductal adenocarcinoma, adenosquamous carcinomas and pancreatic endocrine tumours.

Tumours of the urinary tract include, but are not limited to, bladder, penile, kidney, renal pelvis, ureter, urethral and human papillary renal cancers.

Examples of kidney cancer include, but are not limited to renal cell carcinoma, urothelial cell carcinoma, juxtaglomerular cell tumour (reninoma), angiomyolipoma, renal oncocytoma, Bellini duct carcinoma, clear-cell sarcoma of the kidney, mesoblastic nephroma and Wilms' tumour.

Examples of bladder cancer include, but are not limited to transitional cell carcinoma, squamous cell carcinoma, adenocarcinoma, sarcoma and small cell carcinoma.

Eye cancers include, but are not limited to, intraocular melanoma and retinoblastoma.

Examples of liver cancers include, but are not limited to, hepatocellular carcinoma (liver cell carcinomas with or without fibrolamellar variant), cholangiocarcinoma (intrahepatic bile duct carcinoma), and mixed hepatocellular cholangiocarcinoma. Skin cancers include, but are not limited to, squamous cell carcinoma, Kaposi's sarcoma, malignant melanoma, Merkel cell skin cancer, and non-melanoma skin cancer.

Head-and-neck cancers include, but are not limited to, squamous cell cancer of the head and neck, laryngeal, hypopharyngeal, nasopharyngeal, oropharyngeal cancer, salivary gland cancer, lip and oral cavity cancer and squamous cell.

Lymphomas include, but are not limited to, AIDS-related lymphoma, non-Hodgkin's lymphoma, cutaneous T-cell lymphoma, Burkitt lymphoma, Hodgkin's disease, and lymphoma of the central nervous system.

Sarcomas include, but are not limited to, sarcoma of the soft tissue, osteosarcoma, malignant fibrous histiocytoma, lymphosarcoma, and rhabdomyosarcoma.

Leukemias include, but are not limited to, acute myeloid leukemia, acute lymphoblastic leukemia, chronic lymphocytic leukemia, chronic myelogenous leukemia, and hairy cell leukemia.

The term “treating” or “treatment” as stated throughout this document is used conventionally, for example the management or care of a subject for the purpose of combating, alleviating, reducing, relieving, improving the condition of a disease or disorder, such as a carcinoma.

The compounds of the present invention can be used in particular in therapy and prevention, i.e. prophylaxis, of tumour growth and metastases, especially in solid tumours of all indications and stages with or without pre-treatment of the tumour growth.

Generally, the use of chemotherapeutic agents and/or anti-cancer agents in combination with a compound or pharmaceutical composition of the present invention will serve to:
1. yield better efficacy in reducing the growth of a tumour or even eliminate the tumour as compared to administration of either agent alone,
2. provide for the administration of lesser amounts of the administered chemo-therapeutic agents,
3. provide for a chemotherapeutic treatment that is well tolerated in the patient with fewer deleterious pharmacological complications than observed with single agent chemotherapies and certain other combined therapies,
4. provide for treating a broader spectrum of different cancer types in mammals, especially humans,
5. provide for a higher response rate among treated patients,
6. provide for a longer survival time among treated patients compared to standard chemotherapy treatments,
7. provide a longer time for tumour progression, and/or
8. yield efficacy and tolerability results at least as good as those of the agents used alone, compared to known instances where other cancer agent combinations produce antagonistic effects.

In addition, the compounds of general formula (I) of the present invention can also be used in combination with radiotherapy and/or surgical intervention.

In a further embodiment of the present invention, the compounds of general formula (I) of the present invention may be used to sensitize a cell to radiation, i.e. treatment of a cell with a compound of the present invention prior to radiation treatment of the cell renders the cell more susceptible to DNA damage and cell death than the cell would be in the absence of any treatment with a compound of the present invention. In one aspect, the cell is treated with at least one compound of general formula (I) of the present invention.

Thus, the present invention also provides a method of killing a cell, wherein a cell is administered one or more compounds of the present invention in combination with conventional radiation therapy.

The present invention also provides a method of rendering a cell more susceptible to cell death, wherein the cell is treated with one or more compounds of general formula (I) of the present invention prior to the treatment of the cell to cause or induce cell death. In one aspect, after the cell is treated with one or more compounds of general formula (I) of the present invention, the cell is treated with at least one compound, or at least one method, or a combination thereof, in order to cause DNA damage for the purpose of inhibiting the function of the normal cell or killing the cell.

In other embodiments of the present invention, a cell is killed by treating the cell with at least one DNA damaging agent, i.e. after treating a cell with one or more compounds of general formula (I) of the present invention to sensitize the cell to cell death, the cell is treated with at least one DNA damaging agent to kill the cell. DNA damaging agents useful in the present invention include, but are not limited to, chemotherapeutic agents (e.g. cis platin), ionizing radiation (X-rays, ultraviolet radiation), carcinogenic agents, and mutagenic agents.

In other embodiments, a cell is killed by treating the cell with at least one method to cause or induce DNA damage. Such methods include, but are not limited to, activation of a cell signalling pathway that results in DNA damage when the pathway is activated, inhibiting of a cell signalling pathway that results in DNA damage when the pathway is inhibited, and inducing a biochemical change in a cell, wherein the change results in DNA damage. By way of a non-limiting example, a DNA repair pathway in a cell can be inhibited, thereby preventing the repair of DNA damage and resulting in an abnormal accumulation of DNA damage in a cell.

In one aspect of the invention, a compound of general formula (I) of the present invention is administered to a cell prior to the radiation or other induction of DNA damage in the cell. In another aspect of the invention, a compound of general formula (I) of the present invention is administered to a cell concomitantly with the radiation or other induction of DNA damage in the cell. In yet another aspect of the invention, a compound of general formula (I) of the present invention is administered to a cell immediately after radiation or other induction of DNA damage in the cell has begun.

In another aspect, the cell is in vitro. In another embodiment, the cell is in vivo.

The compounds of the present invention can be administered as the sole pharmaceutical agent or in combination with one or more other pharmaceutically active ingredients where the combination causes no unacceptable adverse effects. The present invention also covers such pharmaceutical combinations. For example, the compounds of the present invention can be combined with: ¹³¹I-chTNT, abarelix, abiraterone, aclarubicin, adalimumab, ado-trastuzumab emtansine, afatinib, aflibercept, aldesleukin, alectinib, alemtuzumab, alendronic acid, alitretinoin, altretamine, amifostine, aminoglutethimide, hexyl aminolevulinate, amrubicin, amsacrine, anastrozole, ancestim, anethole dithiolethione, anetumab ravtansine, angiotensin II, antithrombin III, aprepitant, arcitumomab, arglabin, arsenic trioxide, asparaginase, atezolizumab, axitinib, azacitidine, basiliximab, belotecan, bendamustine, besilesomab, belinostat, bevacizumab, bexarotene, bicalutamide, bisantrene, bleomycin, blinatumomab, bortezomib, buserelin, bosutinib, brentuximab vedotin, busulfan, cabazitaxel, cabozantinib, calcitonine, calcium folinate, calcium levofolinate, capecitabine, capromab, carbamazepine carboplatin, carboquone, carfilzomib, carmofur, carmustine, catumaxomab, celecoxib, celmoleukin, ceritinib, cetuximab, chlorambucil, chlormadinone, chlormethine, cidofovir, cinacalcet, cisplatin, cladribine, clodronic acid, clofarabine, cobimetinib, copanlisib, crisantaspase, crizotinib, cyclophosphamide, cyproterone, cytarabine, dacarbazine, dactinomycin, daratumumab, darbepoetin alfa, dabrafenib, dasatinib, daunorubicin, decitabine, degarelix, denileukin diftitox, denosumab, depreotide, deslorelin, dianhydrogalactitol, dexrazoxane, dibrospidium chloride, dianhydrogalactitol, diclofenac, dinutuximab, docetaxel, dolasetron, doxifluridine, doxorubicin, doxorubicin+estrone, dronabinol, eculizumab, edrecolomab, elliptinium acetate, elotuzumab, eltrombopag, endostatin, enocitabine, enzalutamide, epirubicin, epitiostanol, epoetin alfa, epoetin beta, epoetin zeta, eptaplatin, eribulin, erlotinib, esomeprazole, estradiol, estramustine, ethinylestradiol, etoposide, everolimus, exemestane, fadrozole, fentanyl, filgrastim, fluoxymesterone, floxuridine, fludarabine, fluorouracil, flutamide, folinic acid, formestane, fosaprepitant, fotemustine, fulvestrant, gadobutrol, gadoteridol, gadoteric acid meglumine, gadoversetamide, gadoxetic acid, gallium nitrate, ganirelix, gefitinib, gemcitabine, gemtuzumab, Glucarpidase, glutoxim, GM-CSF, goserelin, granisetron, granulocyte colony stimulating factor, histamine dihydrochloride, histrelin, hydroxycarbamide, I-125 seeds, lansoprazole, ibandronic acid, ibritumomab tiuxetan, ibrutinib, idarubicin, ifosfamide, imatinib, imiquimod, improsulfan, indisetron, incadronic acid, ingenol mebutate, interferon alfa, interferon beta, interferon gamma, iobitridol, iobenguane (¹²³I), iomeprol, ipilimumab, irinotecan, Itraconazole, ixabepilone, ixazomib, lanreotide, lansoprazole, lapatinib, lasocholine, lenalidomide, lenvatinib, lenograstim, lentinan, letrozole, leuprorelin, levamisole, levonorgestrel, levothyroxine sodium, lisuride, lobaplatin, lomustine, lonidamine, masoprocol, medroxyprogesterone, megestrol, melarsoprol, melphalan, mepitiostane, mercaptopurine, mesna, methadone, methotrexate, methoxsalen, methylaminolevulinate, methylprednisolone, methyltestosterone, metirosine, mifamurtide, miltefosine, miriplatin, mitobronitol, mitoguazone, mitolactol, mitomycin, mitotane, mitoxantrone, mogamulizumab, molgramostim, mopidamol, morphine hydrochloride, morphine sulfate, nabilone, nabiximols, nafarelin, naloxone+pentazocine, naltrexone, nartograstim, necitumumab, nedaplatin, nelarabine, neridronic acid, netupitant/palonosetron, nivolumab, pentetreotide, nilotinib, nilutamide, nimorazole, nimotuzumab, nimustine, nintedanib, nitracrine, nivolumab, obinutuzumab, octreotide, ofatumumab, olaparib, olaratumab, omacetaxine mepesuccinate, omeprazole, ondansetron, oprelvekin, orgotein, orilotimod, osimertinib, oxaliplatin, oxycodone, oxymetholone, ozogamicine, p53 gene therapy, paclitaxel, palbociclib, palifermin, palladium-103 seed, palonosetron, pamidronic acid, panitumumab, panobinostat, pantoprazole, pazopanib, pegaspargase, PEG-epoetin beta (methoxy PEG-epoetin beta), pembrolizumab, pegfilgrastim, peginterteron alfa-2b, pembrolizumab, pemetrexed, pentazocine, pentostatin, peplomycin, Perflubutane, perfosfamide, Pertuzumab, picibanil, pilocarpine, pirarubicin, pixantrone, plerixafor, plicamycin, poliglusam, polyestradiol phosphate, polyvinylpyrrolidone+sodium hyaluronate, polysaccharide-K, pomalidomide, ponatinib, porfimer sodium, pralatrexate, prednimustine, prednisone, procarbazine, procodazole, propranolol, quinagolide, rabeprazole, racotumomab, radium-223 chloride, radotinib, raloxifene, raltitrexed, ramosetron, ramucirumab, ranimustine, rasburicase, razoxane, refametinib, regorafenib, risedronic acid, rhenium-186 etidronate, rituximab, rolapitant, romidepsin, romiplostim, romurtide, roniciclib, samarium (¹⁵³Sm) lexidronam, sargramostim, satumomab, secretin, siltuximab, sipuleucel-T, sizofiran, sobuzoxane, sodium glycididazole, sonidegib, sorafenib, stanozolol, streptozocin, sunitinib, talaporfin, talimogene laherparepvec, tamibarotene, tamoxifen, tapentadol, tasonermin, teceleukin, technetium (^{99m}Tc) nofetumomab merpentan, ^{99m}Tc-HYNIC-[Tyr3]-octreotide, tegafur, tegafur+gimeracil+oteracil, temoporfin, temozolomide, temsirolimus, teniposide, testosterone, tetrofosmin, thalidomide, thiotepa, thymalfasin, thyrotropin alfa, tioguanine, tocilizumab, topotecan, toremifene, tositumomab, trabectedin, trametinib, tramadol, trastuzumab, trastuzumab emtansine, treosulfan, tretinoin, trifluridine+tipiracil, trilostane, triptorelin, trametinib, trofosfamide, thrombopoietin, tryptophan, ubenimex, valatinib, valrubicin, vandetanib, vapreotide, vemurafenib, vinblastine, vincristine, vindesine, vinflunine, vinorelbine, vismodegib, vorinostat, vorozole, yttrium-90 glass microspheres, zinostatin, zinostatin stimalamer, zoledronic acid, zorubicin.

The compounds of the invention can further be combined with other reagents targeting the immune system, such as immune checkpoint inhibitors, e.g. aPD-1/-L1 axis antagonists.

PD-1, along with its ligands PD-L1 and PD-L2, function as negative regulators of T cell activation. MAP4K1 suppresses immune cell function. PD-L1 is overexpressed in many cancers and overexpression of PD-1 often occurs concomitantly in tumor infiltrating T cells. Thus results in attenuation of T cell activation and evasion of immune surveillance, which contributes to impaired antitumor immune responses. (Keir M E et al. (2008) Annu. Rev. Immunol. 26:677).

In addition, the inventive compounds can also be used as a therapeutic in a variety of other disorders wherein MAP4K1 is involved such as, cardiovascular and lung diseases.

Accordingly, the compounds according to the invention are suitable for the treatment and/or prophylaxis in particular of cardiovascular, inflammatory and fibrotic disorders and of renal disorders, in particular of acute and chronic renal insufficiency, and also of acute and chronic renal failure.

Accordingly, the compounds according to the invention can be used in medicaments for the treatment and/or prophylaxis of cardiovascular, inflammatory and fibrotic disorders, renal disorders, in particular of acute and chronic renal insufficiency, and also of acute and chronic renal failure.

For the purpose of the present invention the term renal insufficiency comprises both acute and chronic manifestations of renal insufficiency, and also underlying or related renal disorders such as diabetic and non-diabetic nephropathies, hypertensive nephropathies, ischaemic renal disorders, renal hypoperfusion, intradialytic hypotension, obstructive uropathy, renal stenoses, glomerulopathies, glomerulonephritis (such as, for example, primary glomerulonephritides; minimal change glomerulonephritis (lipoidnephrosis); membranous glomerulonephritis; focal segmental glomerulosclerosis (FSGS); membrane-proliferative glomerulonephritis; crescentic glomerulonephritis; mesangioproliterative glomerulonephritis (IgA nephritis, Berger's disease); post-infectious glomerulonephritis; secondary glomerulonephritides: diabetes mellitus, lupus erythematosus, amyloidosis, Goodpasture syndrome, Wegener granulomatosis, Henoch-Schonlein purpura, microscopic polyangiitis, acute glomerulonephritis, pyelonephritis (for example as a result of: urolithiasis, benign prostate hyperplasia, diabetes, malformations, abuse of analgesics, Crohn's disease), glomerulosclerosis, arteriolonecrose of the kidney, tubulointerstitial diseases, nephropathic disorders such as primary and congenital or acquired renal disorder, Alport syndrome, nephritis, immunological kidney disorders such as kidney transplant rejection and immunocomplex-induced renal disorders, nephropathy induced by toxic substances, nephropathy induced by contrast agents, diabetic and non-diabetic nephropathy, renal cysts, nephrosclerosis, hypertensive nephrosclerosis and nephrotic syndrome which can be characterized diagnostically, for example by abnormally reduced creatinine and/or water excretion, abnormally elevated blood concentrations of urea, nitrogen, potassium and/or creatinine, altered activity of renal enzymes, for example glutamyl synthetase, altered urine osmolarity or urine volume, elevated microalbuminuria, macroalbuminuria, lesions on glomerulae and arterioles, tubular dilatation, hyperphosphataemia and/or the need for dialysis. The present invention also comprises the use of the compounds according to the invention for the treatment and/or prophylaxis of sequelae of renal insufficiency, for example pulmonary oedema, heart failure, uremia, anemia, electrolyte disturbances (for example hypercalemia, hyponatremia) and disturbances in bone and carbohydrate metabolism.

The present invention also comprises the use of the compounds according to the invention for the treatment and/or prevention of sequelae of renal insufficiency, for example pulmonary oedema, heart failure, uraemia, anaemia, electrolyte disturbances (for example hyperkalaemia, hyponatraemia) and disturbances in bone and carbohydrate metabolism.

The compounds according to the invention are further suitable for the treatment and/or prevention of polycystic kidney disease (PCKD) and of the syndrome of inappropriate ADH secretion (SIADH).

Furthermore, the compounds according to the invention are also suitable for the treatment and/or prophylaxis of metabolic syndrome, hypertension, resistant hypertension, acute and chronic heart failure, coronary heart disease, stable and unstable angina pectoris, peripheral and cardiac vascular disorders, arrhythmias, atrial and ventricular arrhythmias and impaired conduction, for example atrioventricular blocks degrees I-111 (AB block I-111), supraventricular tachyarrhythmia, atrial fibrillation, atrial flutter, ventricular fibrillation, ventricular flutter, ventricular tachyarrhythmia, Torsade de pointes tachycardia, atrial and ventricular extrasystoles, AV-junctional extrasystoles, sick sinus syndrome, syncopes, AV-nodal re-entry tachycardia, Wolff-Parkinson-White syndrome, of acute coronary syndrome (ACS), autoimmune cardiac disorders (pericarditis, endocarditis, valvolitis, aortitis, cardiomyopathies), shock such as cardiogenic shock, septic shock and anaphylactic shock, aneurysms, boxer cardiomyopathy (premature ventricular contraction (PVC)), for treatment and/or prophylaxis of thromboembolic disorders and ischaemias such as myocardial ischaemia, myocardial infarction, stroke, cardiac hypertrophy, transient and ischaemic attacks, preeclampsia, inflammatory cardiovascular disorders, spasms of the coronary arteries and peripheral arteries, oedema formation, for example pulmonary oedema, cerebral oedema, renal oedema or oedema caused by heart failure, peripheral circulatory disturbances, reperfusion damage, arterial and venous thromboses, myocardial insufficiency, endothelial dysfunction, to prevent restenoses, for example after thrombolysis therapies, percutaneous transluminal angioplasties (PTA), transluminal coronary angioplasties (PTCA), heart transplants and bypass operations, and also micro- and macrovascular damage (vasculitis), increased levels of fibrinogen and of low-density lipoprotein (LDL) and increased concentrations of plasminogen activator inhibitor 1 (PAI-1), and also for treatment and/or prophylaxis of erectile dysfunction and female sexual dysfunction.

In addition, the compounds according to the invention are also suitable for treatment and/or prophylaxis of asthmatic disorders, pulmonary arterial hypertension (PAH) and other forms of pulmonary hypertension (PH) including left-heart disease, HIV, sickle cell anaemia, thromboembolisms (CTEPH), sarcoidosis, COPD or pulmonary fibrosis-associated pulmonary hypertension, chronic-obstructive pulmonary disease (COPD), acute respiratory distress syndrome (ARDS), acute lung injury (ALI), alpha-1-antitrypsin deficiency (AATD), pulmonary fibrosis, pulmonary emphysema (for example pulmonary emphysema induced by cigarette smoke) and cystic fibrosis (CF).

The compounds described in the present invention are also active compounds for control of central nervous system disorders characterized by disturbances of the NO/cGMP system. They are suitable in particular for improving perception, concentration, learning or memory after cognitive impairments like those occurring in particular in association with situations/diseases/syndromes such as mild cognitive impairment, age-associated learning and memory impairments, age-associated memory losses, vascular dementia, craniocerebral trauma, stroke, dementia occurring after strokes (post stroke dementia), post-traumatic craniocerebral trauma, general concentration impairments, concentration impairments in children with learning and memory problems, Alzheimer's disease, Lewy body dementia, dementia with degeneration of the frontal lobes including Pick's syndrome, Parkinson's disease, progressive dementia with corticobasal degeneration, amyolateral sclerosis (ALS), Huntington's disease, demyelinization, multiple sclerosis, thalamic degeneration, Creutzfeld-Jacob dementia, HIV dementia, schizophrenia with dementia or Korsakoff's psychosis. They are also suitable for treatment and/or prophylaxis of central nervous system disorders such as states of anxiety, tension and depression, CNS-related sexual dysfunctions and sleep disturbances, and for controlling pathological disturbances of the intake of food, stimulants and addictive substances.

The compounds according to the invention are furthermore also suitable for controlling cerebral blood flow and thus represent effective agents for controlling migraines. They are also suitable for the prophylaxis and control of sequelae of cerebral infarction (cerebral apoplexy) such as stroke, cerebral ischaemia and craniocerebral trauma. The compounds according to the invention can likewise be used for controlling states of pain and tinnitus.

In addition, the compounds according to the invention have anti-inflammatory action and can therefore be used as anti-inflammatory agents for treatment and/or prophylaxis of sepsis (SIRS), multiple organ failure (MODS, MOF), inflammatory disorders of the kidney, chronic intestinal inflammations (IBD, Crohn's disease, UC), pancreatitis, peritonitis, rheumatoid disorders, inflammatory skin disorders and inflammatory eye disorders.

Furthermore, the compounds according to the invention can also be used for treatment and/or prophylaxis of autoimmune diseases.

The compounds according to the invention are also suitable for treatment and/or prophylaxis of fibrotic disorders of the internal organs, for example the lung, the heart, the kidney, the bone marrow and in particular the liver, and also dermatological fibroses and fibrotic eye disorders. In the context of the present invention, the term fibrotic disorders includes in particular the following terms: hepatic fibrosis, cirrhosis of the liver, pulmonary fibrosis, endomyocardial fibrosis, nephropathy, glomerulonephritis, interstitial renal fibrosis, fibrotic damage resulting from diabetes, bone marrow fibrosis and similar fibrotic disorders, scleroderma, morphea, keloids, hypertrophic scarring (also following surgical procedures), naevi, diabetic retinopathy, proliferative vitroretinopathy and disorders of the connective tissue (for example sarcoidosis).

The compounds according to the invention are also suitable for controlling postoperative scarring, for example as a result of glaucoma operations.

The compounds according to the invention can also be used cosmetically for ageing and keratinized skin.

Moreover, the compounds according to the invention are suitable for treatment and/or prophylaxis of hepatitis, neoplasms, osteoporosis, glaucoma and gastroparesis.

The present invention further provides the use of the compounds according to the invention for treatment and/or prophylaxis of disorders, especially the disorders mentioned above.

The present invention further provides the use of the compounds according to the invention for the treatment and/or prophylaxis of chronic renal disorders, acute and chronic renal insufficiency, diabetic, inflammatory or hypertensive nephropaties, fibrotic disorders, cardiac insufficiency, angina pectoris, hypertension, pulmonary hypertension, ischemias, vascular disorders, thromboembolic disorders, arteriosclerosis, sickle cell anemia, erectile dysfunction, benign prostate hyperplasia, dysuria associated with benign prostate hyperplasia, Huntington, dementia, Alzheimer and Creutzfeld-Jakob.

The present invention further provides a method for treatment and/or prophylaxis of disorders, in particular the disorders mentioned above, using an effective amount of at least one of the compounds according to the invention.

The present invention further provides a method for the treatment and/or prophylaxis of chronic renal disorders, acute and chronic renal insufficiency, diabetic, inflammatory or hypertensive nephropathies, fibrotic disorders, cardiac insufficiency, angina pectoris, hypertension, pulmonary hypertension, ischemias, vascular disorders, thromboembolic disorders, arteriosclerosis, sickle cell anemia, erectile dysfunction, benign prostate hyperplasia, dysuria associated with benign prostate hyperplasia, Huntington, dementia, Alzheimer and Creutzfeld-Jakob.

In another embodiment, the inventive compounds can also be used to treat or to prevent uterine fibroids (uterine leiomyoma or uterine myoma) in women.

Uterine fibroids are benign tumors of the myometrium, the smooth muscle layer of the uterus. Uterine fibroids grow slowly during a women's life, and their growth is dependent on the female sexual hormones estradiol and progesterone [Kawaguchi K et al. Immunohistochemical analysis of oestrogen receptors, progesterone receptors and Ki-67 in leiomyoma and myometrium during the menstrual cycle and pregnancy Virchows Arch A Pathol Anat Histopathol. 1991; 419(4):309-15.], therefore the highest prevalence of uterine fibroids with approx. 70% and >80% in white and afro-american women, respectively, is found from 35 years of age onwards to menopause, when they shrink due to reduced hormone levels [Baird D D et al. High cumulative incidence of uterine leiomyoma in black and white women: Ultrasound evidence Am J Obstet Gynecol. 2003 January; 188(1):100-7.]. Approx 30% and 45% of white and afro-american women, respectively, do show clinically relevant symptoms due to their fibroids, which are heavy menstrual bleeding and pain, which is related to the menstrual cycle [David M et al. Myoma-associated pain frequency and intensity: a retrospective evaluation of 1548 myoma patients. Eur J Obstet Gynecol Reprod Biol. 2016 April; 199:137-40]. Heavy menstrual bleeding in this respect is defined by a blood loss of more than 80 mL in a menstrual bleeding period [Fraser I S et al. The FIGO Recommendations on Terminologies and Definitions for Normal and Abnormal Uterine Bleeding, Semin Reprod Med 2011; 29(5): 383-390]. Submucosal position of the uterine fibroids, e.g. those located directly below the endometrium, seems to have an even more severe effect on uterine bleeding, which may result in anemia in affected women [Yang J H et al. Impact of submucous myoma on the severity of anemia. Fertil Steril. 2011 Apr.; 95(5): 1769-72], Furthermore, uterine fibroids, due to their symptoms, do severely affect the quality of life of affected women [Downes E et al. The burden of uterine fibroids in five European countries. Eur J Obstet Gynecol Reprod Biol. 2010 Sep.; 152(1):96-102],

Compounds of the present invention can be utilized to inhibit, block, reduce or decrease MAP4K1 activation by exogenous and/or endogenous ligands for the reduction of tumour growth and the modulation of dysregulated immune responses e.g. to block immunosuppression and increase immune cell activation and infiltration in the context of cancer and cancer immunotherapy; This method comprises administering to a mammal in need thereof, including a human, an amount of a compound of this invention, or a pharmaceutically acceptable salt, isomer, polymorph, metabolite, hydrate, solvate or ester thereof; which is effective to treat the disorder.

The present invention also provides methods of treating a variety of other disorders wherein MAP4K1 is involved such as, but not limited to, disorders with dysregulated immune responses, inflammation, vaccination for infection & cancer, viral infections, obesity and diet-induced obesity, adiposity, metabolic disorders, hepatic steatosis and uterine fibroids.

These disorders have been well characterized in humans, but also exist with a similar etiology in other mammals, and can be treated by administering pharmaceutical compositions of the present invention.

The term “treating” or “treatment” as used in the present text is used conventionally, e.g., the management or care of a subject for the purpose of combating, alleviating, reducing, relieving, improving the condition of a disease or disorder, such as liquid and solid tumours.

In accordance with a further aspect, the present invention covers compounds of general formula (I), as described supra, or stereoisomers, tautomers, N-oxides, hydrates, solvates, and salts thereof, particularly pharmaceutically acceptable salts thereof, or mixtures of same, for use in the treatment or prophylaxis of diseases, in particular cancer or conditions with dysregulated immune responses or other disorders associated with aberrant MAP4K1 signaling.

The pharmaceutical activity of the compounds according to the invention can be explained by their activity as MAP4K1 inhibitors.

In accordance with a further aspect, the present invention covers the use of compounds of general formula (I), as described supra, or stereoisomers, tautomers, N-oxides, hydrates, solvates, and salts thereof, particularly pharmaceutically acceptable salts thereof, or mixtures of same, for the treatment or prophylaxis of diseases, in particular cancer or conditions with dysregulated immune responses or other disorders associated with aberrant MAP4K1 signaling, particularly liquid and solid tumours.

In accordance with a further aspect, the present invention covers the compounds of general formula (I), as described supra, or stereoisomers, tautomers, N-oxides, hydrates, solvates, and salts thereof, particularly pharmaceutically acceptable salts thereof, or mixtures of same, for the use of treatment or prophylaxis of diseases, in particular cancer or conditions with dysregulated immune responses or other disorders associated with aberrant MAP4K1 signaling, particularly liquid and solid tumours.

In accordance with a further aspect, the present invention covers the use of compounds of general formula (I), as described supra, or stereoisomers, tautomers, N-oxides, hydrates, solvates, and salts thereof, particularly pharmaceutically acceptable salts thereof, or mixtures of same, in a method of treatment or prophylaxis of diseases, in particular cancer or conditions with dysregulated immune responses or other disorders associated with aberrant MAP4K1 signaling, particularly liquid and solid tumours.

In accordance with a further aspect, the present invention covers use of a compound of general formula (I), as described supra, or stereoisomers, tautomers, N-oxides, hydrates, solvates, and salts thereof, particularly pharmaceutically acceptable salts thereof, or mixtures of same, for the preparation of a pharmaceutical composition, preferably a medicament, for the prophylaxis or treatment of diseases, in particular cancer or conditions with dysregulated immune responses or other disorders associated with aberrant MAP4K1 signaling, particularly liquid and solid tumours.

In accordance with a further aspect, the present invention covers a method of treatment or prophylaxis of diseases, in particular cancer or conditions with dysregulated immune responses or other disorders associated with aberrant MAP4K1 signaling, particularly liquid and solid tumours, using an effective amount of a compound of general formula (I), as described supra, or stereoisomers, tautomers, N-oxides, hydrates, solvates, and salts thereof, particularly pharmaceutically acceptable salts thereof, or mixtures of same.

In accordance with a further aspect, the present invention covers pharmaceutical compositions, in particular a medicament, comprising a compound of general formula (I), as described supra, or a stereoisomer, a tautomer, an N-oxide, a hydrate, a solvate, a salt thereof, particularly a pharmaceutically acceptable salt, or a mixture of same, and one or more excipients), in particular one or more pharmaceutically acceptable excipient(s). Conventional procedures for preparing such pharmaceutical compositions in appropriate dosage forms can be utilized.

The present invention furthermore covers pharmaceutical compositions, in particular medicaments, which comprise at least one compound according to the invention, conventionally together with one or more pharmaceutically suitable excipients, and to their use for the above mentioned purposes.

It is possible for the compounds according to the invention to have systemic and/or local activity. For this purpose, they can be administered in a suitable manner, such as, for example, via the oral, parenteral, pulmonary, nasal, sublingual, lingual, buccal, rectal, vaginal, dermal, transdermal, conjunctival, otic route or as an implant or stent. For these administration routes, it is possible for the compounds according to the invention to be administered in suitable administration forms.

For oral administration, it is possible to formulate the compounds according to the invention to dosage forms known in the art that deliver the compounds of the invention rapidly and/or in a modified manner, such as, for example, tablets (uncoated or coated tablets, for example with enteric or controlled release coatings that dissolve with a delay or are insoluble), orally-disintegrating tablets, films/wafers, films/lyophylisates, capsules (for example hard or soft gelatine capsules), sugar-coated tablets, granules, pellets, powders, emulsions, suspensions, aerosols or solutions. It is possible to incorporate the compounds according to the invention in crystalline and/or amorphised and/or dissolved form into said dosage forms.

Parenteral administration can be effected with avoidance of an absorption step (for example intravenous, intraarterial, intracardial, intraspinal or intralumbal) or with inclusion of absorption (for example intramuscular, subcutaneous, intracutaneous, percutaneous or intraperitoneal). Administration forms which are suitable for parenteral administration are, inter alia, preparations for injection and infusion in the form of solutions, suspensions, emulsions, lyophylisates or sterile powders.

Examples which are suitable for other administration routes are pharmaceutical forms for inhalation [inter alia powder inhalers, nebulizers], nasal drops, nasal solutions, nasal sprays; tablets/films/wafers/capsules for lingual, sublingual or buccal administration; suppositories; eye drops, eye ointments, eye baths, ocular inserts, ear drops, ear sprays, ear powders, ear-rinses, ear tampons; vaginal capsules, aqueous suspensions (lotions, mixturae agitandae), lipophilic suspensions, emulsions, ointments, creams, transdermal therapeutic systems (such as, for example, patches), milk, pastes, foams, dusting powders, implants or stents.

The compounds according to the invention can be incorporated into the stated administration forms. This can be effected in a manner known per se by mixing with pharmaceutically suitable excipients. Pharmaceutically suitable excipients include, inter alia,
fillers and carriers (for example cellulose, microcrystalline cellulose (such as, for example, Avicel®), lactose, mannitol, starch, calcium phosphate (such as, for example, Di-Cafos®)),
ointment bases (for example petroleum jelly, paraffins, triglycerides, waxes, wool wax, wool wax alcohols, lanolin, hydrophilic ointment, polyethylene glycols),
bases for suppositories (for example polyethylene glycols, cacao butter, hard fat),
solvents (for example water, ethanol, isopropanol, glycerol, propylene glycol, medium chain-length triglycerides fatty oils, liquid polyethylene glycols, paraffins),
surfactants, emulsifiers, dispersants or wetters (for example sodium dodecyl sulfate), lecithin, phospholipids, fatty alcohols (such as, for example, Lanette®), sorbitan fatty acid esters (such as, for example, Span®), polyoxyethylene sorbitan fatty acid esters (such as, for example, Tween®), polyoxyethylene fatty acid glycerides (such as, for example, Cremophor®), polyoxethylene fatty acid esters, polyoxyethylene fatty alcohol ethers, glycerol fatty acid esters, poloxamers (such as, for example, Pluronic®),
buffers, acids and bases (for example phosphates, carbonates, citric acid, acetic acid, hydrochloric acid, sodium hydroxide solution, ammonium carbonate, trometamol, triethanolamine),
isotonicity agents (for example glucose, sodium chloride),
adsorbents (for example highly-disperse silicas),
viscosity-increasing agents, gel formers, thickeners and/or binders (for example polyvinylpyrrolidone, methylcellulose, hydroxypropylmethylcellulose, hydroxypropylcellulose, carboxymethylcellulose-sodium, starch, carbomers, polyacrylic acids (such as, for example, Carbopol®); alginates, gelatine),
disintegrants (for example modified starch, carboxymethylcellulose-sodium, sodium starch glycolate (such as, for example, Explotab®), cross-linked polyvinylpyrrolidone, croscamnellose-sodium (such as, for example, AcDiSol®)),
flow regulators, lubricants, glidants and mould release agents (for example magnesium stearate, stearic acid, talc, highly-disperse silicas (such as, for example, Aerosil®)),
coating materials (for example sugar, shellac) and film formers for films or diffusion membranes which dissolve rapidly or in a modified manner (for example polyvinylpyrrolidones (such as, for example, Kollidon®), polyvinyl alcohol, hydroxypropylmethylcellulose, hydroxypropylcellulose, ethylcellulose, hydroxypropylmethylcellulose phthalate, cellulose acetate, cellulose acetate phthalate, polyacrylates, polymethacrylates such as, for example, Eudragit®)),
capsule materials (for example gelatine, hydroxypropylmethylcellulose),
synthetic polymers (for example polylactides, polyglycolides, polyacrylates, polymethacrylates (such as, for example, Eudragit®), polyvinylpyrrolidones (such as, for example, Kollidon®), polyvinyl alcohols, polyvinyl acetates, polyethylene oxides, polyethylene glycols and their copolymers and blockcopolymers),
plasticizers (for example polyethylene glycols, propylene glycol, glycerol, triacetine, triacetyl citrate, dibutyl phthalate),
penetration enhancers,
stabilisers (for example antioxidants such as, for example, ascorbic acid, ascorbyl palmitate, sodium ascorbate, butylhydroxyanisole, butylhydroxytoluene, propyl gallate),
preservatives (for example parabens, sorbic acid, thiomersal, benzalkonium chloride, chlorhexidine acetate, sodium benzoate),
colourants (for example inorganic pigments such as, for example, iron oxides, titanium dioxide),
flavourings, sweeteners, flavour- and/or odour-masking agents.

The present invention furthermore relates to a pharmaceutical composition which comprise at least one compound according to the invention, conventionally together with one or more pharmaceutically suitable excipient(s), and to their use according to the present invention.

In accordance with another aspect, the present invention covers pharmaceutical combinations, in particular medicaments, comprising at least one compound of general formula (I) of the present invention and at least one or more further active ingredients, in particular for the treatment and/or prophylaxis of cancer or conditions with dysregulated immune responses or other disorders associated with aberrant MAP4K1 signaling generic name disorders, particularly liquid and solid tumours.

The term “combination” in the present invention is used as known to persons skilled in the art, it being possible for said combination to be a fixed combination, a non-fixed combination or a kit-of-parts.

A “fixed combination” in the present invention is used as known to persons skilled in the art and is defined as a combination wherein, for example, a first active ingredient, such as one or more compounds of general formula (I) of the present invention, and a further active ingredient are present together in one unit dosage or in one single entity.

One example of a “fixed combination” is a pharmaceutical composition wherein a first active ingredient and a further active ingredient are present in admixture for simultaneous administration, such as in a formulation. Another example of a “fixed combination” is a pharmaceutical combination wherein a first active ingredient and a further active ingredient are present in one unit without being in admixture.

A non-fixed combination or “kit-of-parts” in the present invention is used as known to persons skilled in the art and is defined as a combination wherein a first active ingredient and a further active ingredient are present in more than one unit. One example of a non-fixed combination or kit-of-parts is a combination wherein the first active ingredient and the further active ingredient are present separately. It is possible for the components of the non-fixed combination or kit-of-parts to be administered separately, sequentially, simultaneously, concurrently or chronologically staggered.

Based upon standard laboratory techniques known to evaluate compounds useful for the treatment of cancer or conditions with dysregulated immune responses or other disorders associated with aberrant MAP4K1 signaling, by standard toxicity tests and by standard pharmacological assays for the determination of treatment of the conditions identified above in mammals, and by comparison of these results with the results of known active ingredients or medicaments that are used to treat these conditions, the effective dosage of the compounds of the present invention can readily be determined for treatment of each desired indication. The amount of the active ingredient to be administered in the treatment of one of these conditions can vary widely according to such considerations as the particular compound and dosage unit employed, the mode of administration, the period of treatment, the age and sex of the patient treated, and the nature and extent of the condition treated.

The total amount of the active ingredient to be administered will generally range from about 0.001 mg/kg to about 200 mg/kg body weight per day, and preferably from about 0.01 mg/kg to about 20 mg/kg body weight per day. Clinically useful dosing schedules will range from one to three times a day dosing to once every four weeks dosing. In addition, it is possible for “drug holidays”, in which a patient is not dosed with a drug for a certain period of time, to be beneficial to the overall balance between pharmacological effect and tolerability. It is possible for a unit dosage to contain from about 0.5 mg to about 1500 mg of active ingredient, and can be administered one or more times per day or less than once a day. The average daily dosage for administration by injection, including intravenous, intramuscular, subcutaneous and parenteral injections, and use of infusion techniques will preferably be from 0.01 to 200 mg/kg of total body weight. The average daily rectal dosage regimen will preferably be from 0.01 to 200 mg/kg of total body weight. The average daily vaginal dosage regimen will preferably be from 0.01 to 200 mg/kg of total body weight. The average daily topical dosage regimen will preferably be from 0.1 to 200 mg administered between one to four times daily. The transdermal concentration will preferably be that required to maintain a daily dose of from 0.01 to 200 mg/kg. The average daily inhalation dosage regimen will preferably be from 0.01 to 100 mg/kg of total body weight.

Of course the specific initial and continuing dosage regimen for each patient will vary according to the nature and severity of the condition as determined by the attending diagnostician, the activity of the specific compound employed, the age and general condition of the patient, time of administration, route of administration, rate of excretion of the drug, drug combinations, and the like. The desired mode of treatment and number of doses of a compound of the present invention or a pharmaceutically acceptable salt or ester or composition thereof can be ascertained by those skilled in the art using conventional treatment tests.

### EXPERIMENTAL SECTION

NMR peak forms are stated as they appear in the spectra, possible higher order effects have not been considered. The multiplicities are stated according to the signal form which appears in the spectrum, NMR-spectroscopic effects of a higher order were not taken into consideration. Multiplicity of the NMR signals: s=singlet, d=doublet, t=triplet, q=quartet,

quin=quintet, br=broad signal, m=multiplet. NMR signals: shift in [ppm]. Combinations of multiplicity could be e.g. dd=doublet from doublet.

In some cases not all H atoms are found as a signal in the NMR because the signal could overlay with a solvent signal or is a very broad signal dependent on the NMR solvent used.

Chemical names were generated using the ACD/Name software from ACD/Labs. In some cases generally accepted names of commercially available reagents were used in place of ACD/Name generated names.

Table 1 lists the abbreviations used in this paragraph and in the Examples section as far as they are not explained within the text body. Other abbreviations have their meanings customary per se to the skilled person.

TABLE 1
Abbreviations

| | |
|---|---|
| PBMCs | Peripheral blood mononuclear cells |
| AUC | Area Under Curve |
| DCM | dichloromethane |
| DMF | N,N-dimethylformamide |
| DMSO | dimethylsulphoxide |
| EAE | experimental autoimmune encephalomyelitis |
| EDTA | Ethylenediaminetetraacetic acid |
| h | hour |
| FCS | fetal calf serum |
| HMDS | Hexamethyldisilazane |
| LPS | lipopolysaccharide |
| mCPBA | 3-chloroperoxybenzoic acid |
| mL | milliliter |
| μL | microliter |
| min. | minute(s) |
| MW | microwave |
| PBMC | peripheral blood mononuclear cells |
| RT or rt | room temperature |
| sat. | saturated |
| SDS | Sodium dodecyl sulfate |
| THF | tetrahydrofuran |
| TNFa | tumour necrosis factor alpha |
| uM | micromolar |
| IFNg | Interferon gamma |

The various aspects of the invention described in this application are illustrated by the following examples which are not meant to limit the invention in any way.

The example testing experiments described herein serve to illustrate the present invention and the invention is not limited to the examples given.

### Experimental Section—General Part

All reagents, for which the synthesis is not described in the experimental part, are either commercially available, or are known compounds or may be formed from known compounds by known methods by a person skilled in the art.

The compounds and intermediates produced according to the methods of the invention may require purification. Purification of organic compounds is well known to the person skilled in the art and there may be several ways of purifying the same compound. In some cases, no purification may be necessary. In some cases, the compounds may be purified by crystallization. In some cases, impurities may be stirred out using a suitable solvent. In some cases, the compounds may be purified by chromatography, particularly flash column chromatography, using for example prepacked silica gel cartridges, e.g. Biotage SNAP cartidges KP-Sil® or KP-NH® in combination with a Biotage autopurifier system (SP4® or Isolera Four®) and eluents such as gradients of hexane/ethyl acetate, DCM/methanol, or DCM/ethanol. In some cases, the compounds may be purified by preparative HPLC using for example a Waters autopurifier equipped with a diode array detector and/or on-line electrospray ionization mass spectrometer in combination with a suitable prepacked reverse phase column and eluents such as gradients of water and acetonitrile which may contain additives such as trifluoroacetic acid, formic acid or aqueous ammonia.

In some cases, purification methods as described above can provide those compounds of the present invention which possess a sufficiently basic or acidic functionality in the form of a salt, such as, in the case of a compound of the present invention which is sufficiently basic, a trifluoroacetate or formate salt for example, or, in the case of a compound of the present invention which is sufficiently acidic, an ammonium salt for example. A salt of this type can either be transformed into its free base or free acid form, respectively, by various methods known to the person skilled in the art, or be used as salts in subsequent biological assays. It is to be understood that the specific form (e.g. salt, free base etc.) of a compound of the present invention as isolated and as described herein is not necessarily the only form in which said compound can be applied to a biological assay in order to quantify the specific biological activity.

### Experimental Section—General Synthesis

The following paragraphs outline a variety of synthetic approaches suitable to prepare compounds of the general formula (I), and intermediates useful for their synthesis. In addition to the routes described below, also other routes may be used to synthesize the target compounds, in accordance with common general knowledge of a person skilled in the art of organic synthesis. The order of transformations exemplified in the following schemes is therefore not intended to be limiting, and suitable synthesis steps from various schemes can be combined to form additional synthesis sequences. In addition, interconversion of any of the substituents, in particular R¹, R^{2a}, R^{2b}, R^{3a}, R^{3b}, R^{4a} and R^{4b}, which are as defined in formula (I) supra, can be achieved before and/or after the exemplified transformations. These modifications can be, for example, the introduction of protective groups, cleavage of protective groups, reduction or oxidation of functional groups, halogenation, metallation, metal catalysed coupling reactions, exemplified by but not limited to e.g. Buchwald, Suzuki, Sonogashira and Ullmann coupling, ester saponifications, amide coupling reactions, and/or substitution or other reactions known to a person skilled in the art. These transformations include those which introduce a functionality allowing for further interconversion of substituents. Appropriate protective groups and their introduction and cleavage are well-known to a person skilled in the art (see for example T. W. Greene and P. G. M. Wuts in Protective Groups in Organic Synthesis, 4^{th} edition, Wiley 2006).

Further, it is possible that two or more successive steps may be performed without work-up being performed between said steps, e.g. a “one-pot” reaction, as it is well-known to a person skilled in the art.

Intermediates of formula (II) where R¹, R^{2a}, R^{2b}, R^{3a}, R^{3b}, R^{4a} and R^{4b} are as defined for the compounds of general formula (I) are cyclized as outlined in Scheme 1 to compounds of general formula (III) by reaction with an appropriate reagent, such as N-(3-dimethylaminopropyl)-N′-ethylcarbodiimide hydrochloride, or p-toluenesulfonyl chloride, in an appropriate solvent such as acetonitrile, methanol, ortetrahydrofuran, optionally in the presence of a base such as for example triethylamine, sodium hydroxide, or potassium hydroxide at a temperature ranging from room temperature to the boiling point of the used solvent. Preferably, for compounds of formula (II), the reaction will be performed in the presence of N-(3-dimethylaminopropyl)-N′-ethylcarbodiimide hydrochloride and trimethylamine in acetonitrile at a temperature of 40° C. Depending on the choice of protecting group PG in compounds of formula (III), which is preferentially trimethylsilylethyloxymethyl (SEM) or toluenesulfonyl (Ts), but can be any other protecting group well known to the person skilled in the art, the deprotection of compounds of general formula (III) with PG=SEM can be performed using trifluoroacetic acid in an inert solvent such as dichloromethane, within a temperature range from 0° C. to the boiling point of the used solvent. Alternatively, the deprotection of compounds of general formula (III) with PG=Ts can be performed using sodium hydroxide or potassium carbonate, in a solvent such as methanol or ethanol, within a temperature range from 0° C. to the boiling point of the used solvent. Preferably the reaction is carried out at room temperature to afford compounds of general formula (I). The deprotection in the case of trimethylsilylethyloxymethyl can be also performed using tetra-butylammonium fluoride in the presence of ethylenediamine in an inert solvent such as tetrahydrofuran within a temperature range from 0° C. to the boiling point of the used solvent to afford compounds of general formula (I). Conversely, compounds of general formula (II) can first be de-protected by the abovementioned conditions to give compounds of general formula (IV) followed by cyclization, also via the aforementioned methods to afford compounds of general formula (I).

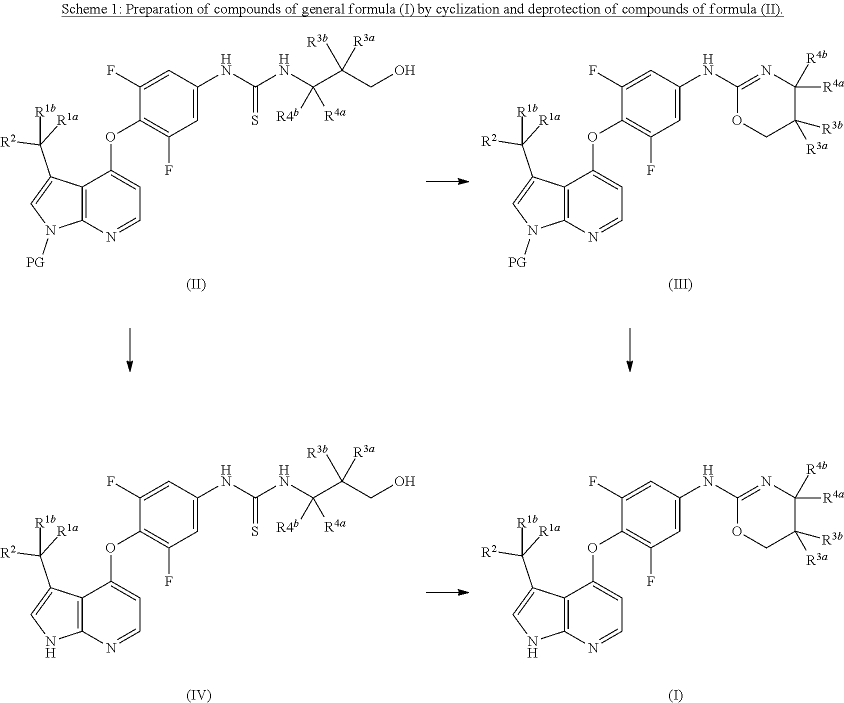

Alternatively, compounds of general formula (III) are synthesized from compounds of general formula (II) via a methylation, cyclization strategy outlined in Scheme 2. First compounds of general formula (II) are reacted with suitable methylating reagents such as iodomethane, dimethyl sulfate, methyl trifluoromethanesulfonate, methyl methanesulfonate, or trimethyloxonium tetrafluoroborate, in solvents such as dichloromethane, tetrahydrofuran, acetone, or acetonitrile, in the presence of bases such as triethylamine, N,N-diisopropylethylamine, or sodium hydride, within a temperature range from 0° C. to the boiling point of the respective solvent. Preferably the reaction is carried out with iodomethane in acetone in the presence of N,N-diisopropylethylamine at 55° C. to afford compounds of general formula (V). Next, compounds of general formula (V) are cyclized by treatment with bases such as sodium hydroxide, or sodium hydride in solvents such as tetrahydrofuran, N,N-dimethylformamide, or dimethyl sulfoxide, within a temperature range from 0° C. to the boiling point of the respective solvent. Preferably, the reaction is carried out with sodium hydroxide in tetrahydrofuran at room temperature to afford compounds of general formula (III).

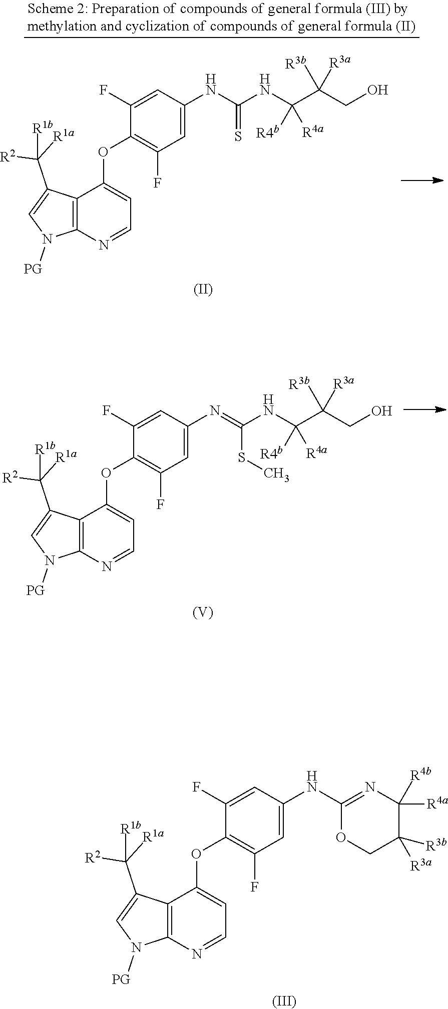

Compounds of general formula (II) can be assembled according to Scheme 3, by reaction of amine derivatives of formula (VI), in which R¹, R^{2a}, R^{2b} are as defined for the compounds of general formula (I), and a second amine derivative (VII), in which R^{3a}, R^{3b}, R^{4a} and R^{4b} are as defined for the compounds of general formula (I), by means of thiourea formation well known to a person skilled in the art. Amines of general formula (VII) are commercially available or synthesized by methods known to one skilled in the art.

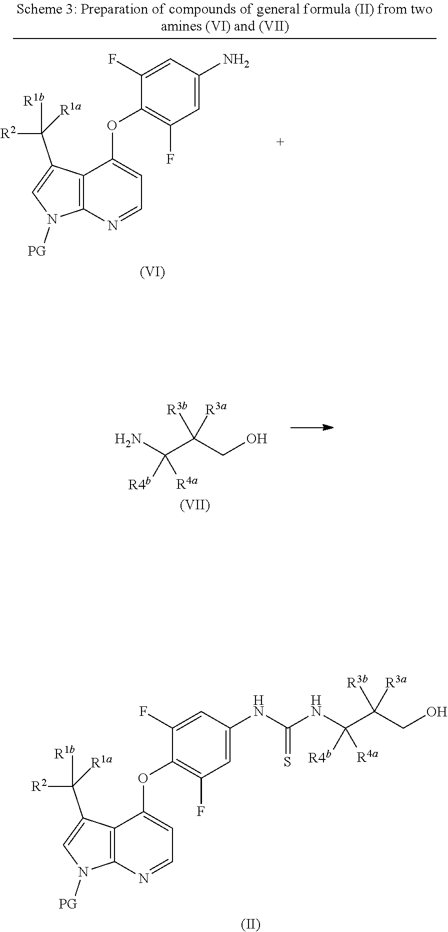

Said thiourea formation can be performed by reaction of compounds of general formula (VI) with the intermediacy of a formed and possibly isolated isothiocyanate (VIII) (Scheme 4) using a suitable reagent such as thiophosgene or 1,1′-thiocarbonylbis-1H-imidazole.

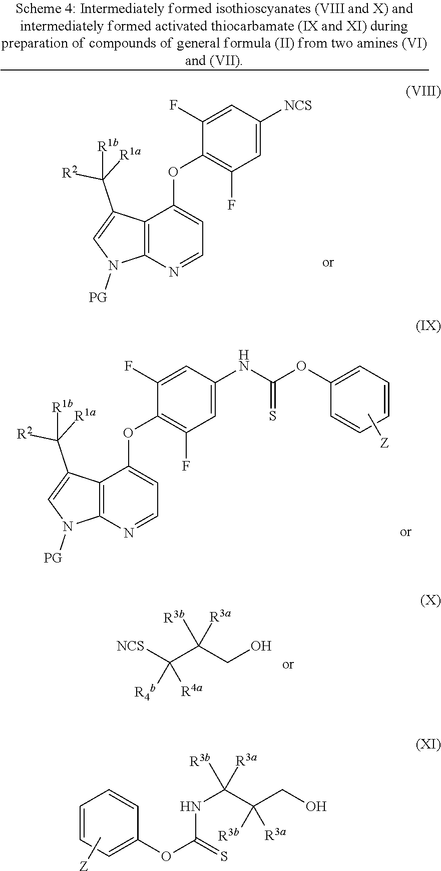

Compounds of general formula (II) can also be assembled by conversion of amine derivatives of formula (VI) to an intermediately formed and possibly isolated thiocarbamate (IX, Scheme 4) in which Z is H, NO₂, or perfluoro using a suitable reagent such as O-phenyl chlorothionoformate in an appropriate solvent such as tetrahydrofuran, dichloromethane, or ethylacetate in the presence of an appropriate base such as pyridine, sodium hydrogencarbonate, or triethylamine. This intermediate (IX) is then reacted with the second amine derivative (VII) in an appropriate solvent such as pyridine, or N,N-dimethylformamide at a temperature between room temperature and the boiling point of the respective solvent. Preferably the reaction is carried out at either 60° C. or 90° C. in N,N-dimethylformamide to afford intermediates of formula (II).

In a similar way the compounds of general formula (II) can be assembled using the amine (VII) as starting material. Using the previously described reaction sequence, amine (VII) can be reacted to the intermediately formed isothiocyanate (X, Scheme 4), if it is not commercially available, in which R^{3a}, R^{3b}, R^{4a} and R^{4b} are as defined for the compounds of general formula (I), or the thiocarbamate (XI, Scheme 4) in which Z is H, NO₂, or perfluoro using a suitable reagent such as O-phenyl chlorothionoformate. This activated intermediate is then reacted with the second amine of general formula (VI) to afford compounds of general formula (II).

The amine intermediates of formula (VI) where R^{1a}, R^{1b}, and R² are as defined for the compounds of general formula (I) are known to one skilled in the art and can be prepared according to Schemes 5 if they are not commercially available. Halo-pyridines of general formula (XII) where X═F, or Cl, are treated with bases such as n-butyllithium, or lithium diisopropylamide, in solvents such as tetrahydrofuran, at −78° C. and subsequently reacted with amides of general formula (XIII), in which R^{1a}, R^{1b}, and R² are as defined for the compounds of general formula (I) which are commercially available or synthesized by methods known to one skilled in the art as described in Org. Process Res. Dev., 2009, 13 (3), pp 576-580. Preferably the reaction is carried out by warming the reaction slowly from −78° C. to room temperature to give compounds of general formula (XIV). Compounds of general formula (XIV) are then converted into the corresponding epoxide of general formula (XV) by methods known to one skilled in the art as reported in Journal of Organic Chemistry, 2006, vol. 71, #15, p. 5538-5545. Compounds of general formula (XIV) are reacted with reagents such as trimethylsulfonium iodide, in the presence of a base such as sodium hydride or potassium tert-butoxide, in a solvent such as dimethylsulfoxide or tetrahydrofuran, at a temperature between 0° C. and the boiling point of the respective solvent. Preferably the reaction is carried out at 15° C. to give compounds of formula (XV). The epoxide function of compounds of general formula (XV) is then opened with ammonia with subsequent cyclization as reported in Journal of Organic Chemistry, 2006, vol. 71, #15, p. 5538-5545. Compounds of general formula (XV) are treated with a solution of ammonia in water, in solvents such as tetrahydrofuran, at temperatures between room temperature and the boiling point of the respective solvent. Preferably the reaction is carried out at 60° C. to give compounds of general formula (XVI). Compounds of general formula (XVI) are then dehydrated with appropriate dehydrating agents such as thionyl chloride, in the presences of a base such as pyridine, in an appropriate solvent such as dichloromethane, at a temperature between room temperature and the boiling point of the respective solvent. Preferably the reaction is carried out at room temperature to afford compounds of general formula (XVII).

Said heterocycle of the formula (XVII) can be converted to heterocycles of formula (XX) via a three-step reaction sequence. First heterocycles of formula (XVII) are reacted with oxidants such as m-chloroperoxybenzoic acid in solvents such as dichloromethane at temperatures between 0° C. and the boiling point of the respective solvents. Preferably the reaction is carried out between 0° C. and 15° C. to afford pyridine N-oxides of formula (XVIII). Compounds of formula (XVIII) are subsequently nitrated by reaction with nitric acid, in the presence of a suitable acidic solvent such a trifluoroacetic acid, concentrated hydrochloric acid or concentrated sulfuric acid, in a temperature range between 0° C. and the boiling point of the respective solvent. The reaction is preferably carried out with nitric acid in trifluoroacetic acid at 0° C. to afford nitrated derivatives of formula (XIX). Derivatives of formula (XIX) are then reacted with reagents such as trichloroacetyl chloride, or acetyl chloride, in the presence of hexamethyldisilazane in solvents such as tetrahydrofuran, in a temperature range between 0° C. and the boiling point of the respective solvent. Preferably, the reaction is carried out between 0° C. and room temperature to afford compounds of formula (XX).

Said heterocycles of formula (XX) can be transformed to the protected intermediates of the formula (XXI) using an appropriate reagent such as trimetylsilylethoxymethyl chloride, triisopropylsilyl chloride, p-toluenesulfonyl chloride, or trityl chloride or other reagents known to a person skilled in the art. Preferably, trimetylsilylethoxymethyl chloride is used, in the presence of a base such as sodium hydride, triethylamine, or N,N-diisopropylethylamine in an inert solvent such as tetrahydrofuran, dimethylsulfoxide or N,N-dimethylformamide. Protected heterocycles of the formula (XXI) are then reacted with a compound of the formula (XXII), which is commercially available or synthesized by methods known to one skilled in the art, in the presence of sodium hydride or an alkali carbonate, such as sodium carbonate, potassium carbonate, or cesium carbonate, in a suitable solvent such as dimethylsulfoxide or N,N-dimethylformamide, at a temperature between room temperature and the boiling point of the respective solvent. Ideally the reaction is carried out at 50° C. to give compounds of formula (XXIII). De-chlorination is then preferentially performed using a hydrogen atmosphere and palladium on carbon as catalyst in an inert solvent such as ethanol, ethyl acetate or dichloromethane at 20-50° C. as described in Org. Process Res. Dev. 2010, page 168-173, to give amines of formula (VI) where R^{1a}, R^{1b}, and R² are as defined for the compounds of general formula (I).

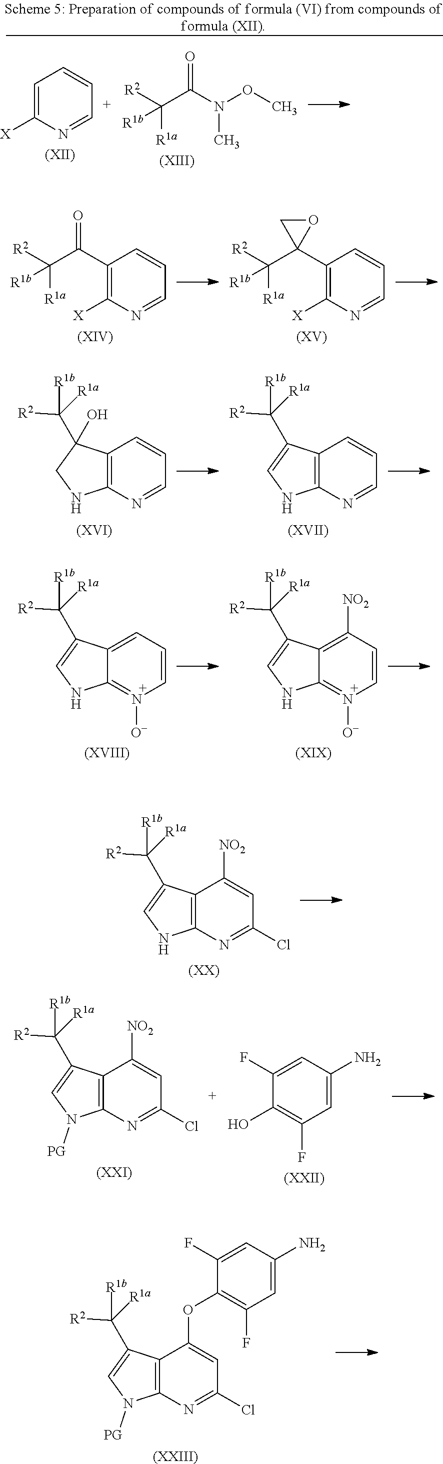

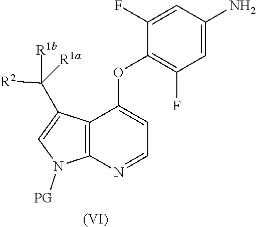

LC-MS methods:

Method 1:

Instrument: Agilent 1200\G6110A; column: Kinetex@ 5 um EVO C18 30*2.1 mm; mobile phase A: 0.0375% trifluoroacetic acid in water (v/v), mobile phase B: 0.01875% trifluoroacetic acid in Acetonitrile (v/v); gradient: 0.01 min 5% B □ 0.80 min 95% B □1.20 min 95% B □ 1.21 min 5% B □ 1.5 min 5% B; flow rate: 1.5 mL/min; oven temperature: 50° C.; UV detection: DAD (220&254 nm).

Method 2:

Instrument: SHIMADZU LCMS-2020; column: Kinetex EVO C18 2.1*30 mm, 5 um; mobile phase A: 0.0375% trifluoroacetic acid in water (v/v), mobile phase B: 0.01875% trifluoroacetic acid in Acetonitrile (v/v); gradient: 0.0 min 30% B □ 3.0 min 90% B □ 3.50 min 90% B □ 3.51 min 30% B □ 4.0 min 30% B; flow rate: 0.8 mL/min; oven temperature: 50° C.; UV detection: DAD (220&254 nm).

Method 3:

Instrument: Waters Acquity UPLCMS SingleQuad; Column: Acquity UPLC BEH C18 1.7 μm, 50×2.1 mm; eluent A: water+0.1 vol % formic acid (99%), eluent B: acetonitrile; gradient: 0-1.6 min 1-99% B, 1.6-2.0 min 99% B; flow 0.8 mL/min; temperature: 60° C.; DAD scan: 210-400 nm.

Method 4:

Instrument: Waters Acquity UPLCMS SingleQuad; Column: Acquity UPLC BEH C18 1.7 μm, 50×2.1 mm; eluent A: water+0.2 vol % aqueous ammonia (32%), eluent B: acetonitrile; gradient: 0-1.6 min 1-99% B, 1.6-2.0 min 99% B; flow 0.8 mL/min; temperature: 60° C.; DAD scan: 210-400 nm.

Method 5:

MS instrument type: Agilent 1200 LC/G1956A MSD; HPLC instrument type: Agilent ChemStation Rev.B.04.03; column: Kinetex EVO C18 2.1×30 mm, 5 μm; mobile phase A: 0.0375% TFA in Water (v/v), mobile phase B: 0.01875% TFA in Acetonitrile (v/v); gradient: 0.01 min 5% B® 0.80 min 95% B® 1.2 min 95% B® 1.21 min 5% B® 1.5 min 5% B; flow rate: 1.5 mL/min; oven temperature: 50° C.; UV detection: 220 nm & 254 nm.

Method 6:

MS instrument type: SHIMADZU LCMS-2020; HPLC instrument type: LabSolutions Version 5.72; column: Chromolith@Flash RP-18E 25-2 MM; mobile phase A: 0.0375% TFA in water (v/v), mobile phase B: 0.01875% TFA in Acetonitrile (v/v); gradient: 0.00 min 0% B® 0.80 min 60% B® 1.20 min 60% B® 1.21 min 0% B® 1.5 min 0% B; flow rate: 1.5 mL/min; oven temperature: 50° C.; UV detection: 220 nm & 254 nm.

### Intermediate 1

### N-methoxy-N-methyl-1-(trifluoromethyl)cyclobutanecarboxamide

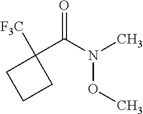

To a mixture of 1-(trifluoromethyl)cyclobutanecarboxylic acid (CAS No: 277756-45-3, 55 g, 327.16 mmol), N,O-dimethylhydroxyiamine hydrochloride (38.5 g, 394.69 mmol) and 1-[bis(dimethylamino)methylene]-1 FI-1,2,3-triazolo[4,5-B]pyridinium-3-oxid-hexafluorophosphate (156.75 g, 412.25 mmol) in dichloromethane (1 L) was added triethylamine (150 mL, 1.08 mol) at room temperature under nitrogen. The mixture was stirred at room temperature for 16 hours. The reaction mixture was concentrated by rotary evaporator under reduced pressure. The residue was diluted with tert-butyl methyl ether (1.5 L) and water (500 mL), and the two phases were separated. The organic phase was washed with 1 M hydrochloric acid (aq., 500 mL×2), sat. aq. sodium hydrogencarbonate (500 mL), brine (500 mL), dried over Sodium sulfate, filtered and concentrated under reduced pressure. The residue was purified by silica gel chromatography (tert-butyl methyl ether) to give N-methoxy-N-methyl-1-(trifluoromethyl)cyclobutanecarboxamide (58 g, 84% yield) as a light yellow oil.

¹H-NMR: (400 MHz, CDCl₃): δ [ppm]=3.68 (s, 3H), 3.24 (s, 3H), 2.70-2.67 (m, 2H), 2.50-2.48 (m, 2H), 2.10-2.07 (m, 1H), 1.82-1.79 (m, 1H).

### Intermediate 2

### (2-fluoro-3-pyridyl)-[1-(trifluoromethyl)cyclobutyl]methanone

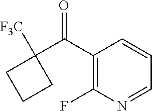 [SMILES: O=C(c1cccnc1F)C1(C(F)(F)F)CCC1]

To a solution of 2,2,6,6-tetramethylpiperidine (110 mL, 647.91 mmol) in tetrahydrofuran (250 mL) was added n-BuLi (250 mL, 2.5 M in hexane) at −70° C. under nitrogen. The mixture was stirred at −70° C. for 1 hour. Then 2-fluoropyridine (CAS No: 372-48-5, 65.56 g, 675.25 mmol) was added drop-wise at −70° C. The mixture was stirred at −70° C. for 1 hour. N-methoxy-N-methyl-1-(trifluoromethyl)cyclobutanecarboxamide (Intermediate 1, 62 g, 293.59 mmol) in tetrahydrofuran (60 mL) was added drop-wise to the mixture at −70° C. The resulting solution was stirred at −70° C. for 2 hours. The reaction mixture was quenched with sat. aq. ammonium chloride (500 mL) at −70° C. The resulting mixture was extracted with tert-butyl methyl ether (2 L). The organic phase was washed with water (400 mL×2), 1 M Sodium dihydrogen phosphate (aq., 400 mL×2), brine (500 mL), dried over sodium sulfate, filtered and concentrated under reduced pressure to give 100 g of crude product. This product was combined with another batch of crude product (20 g). The combined crude product was purified by silica gel chromatography (petroleum ether: ethyl acetate=30:1) to give (2-fluoro-3-pyridyl)-[1-(trifluoromethyl)cyclobutyl]methanone (76 g) as a yellow oil.

LC-MS (Method 1): Rt=0.82 min; m/z=248.0 [M+H]⁺.

¹H-NMR: (400 MHz, CDCl₃): δ [ppm]=8.40-8.38 (m, 1H), 8.04-8.03 (m, 1H), 7.33-7.30 (m, 1H), 2.84-2.81 (m, 2H), 2.65-2.61 (m, 2H), 2.16-1.94 (m, 1H), 1.93-1.89 (m, 1H).

### Intermediate 3

### 2-fluoro-3-[2-[1-(trifluoromethyl)cyclobutyl]oxiran-2-yl]pyridine

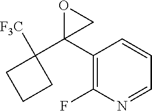 [SMILES: Fc1ncccc1C1(C2(C(F)(F)F)CCC2)CO1]

Sodium hydride (20.39 g, 509.73 mmol, 60% purity) was suspended in dimethyl sulfoxide (800 mL) at room temperature under nitrogen. The mixture was stirred at 65° C. for 1 hour. The mixture was then cooled to room temperature, and trimethylsulfoxonium iodide (117.6 g, 534.37 mmol) was added in portions to the mixture, which was stirred at room temperature for 1 hour. (2-fluoro-3-pyridyl)-[1-(trifluoromethyl)cyclobutyl]methanone (Intermediate 2, 60 g, 242.73 mmol) was added, and the reaction was allowed to proceed at room temperature for 2 hours. The reaction was quenched with ice-water (1 L). The resulting mixture was extracted with ethyl acetate (1 L×2). The combined organic phase was washed with water (500 mL), brine (500 mL), dried over sodium sulfate, filtered and concentrated under reduced pressure to give 80 g of crude product. This crude product was combined with another batch of crude product (20 g). The combined crude product was purified by silica gel chromatography (petroleum ether: ethyl acetate=10:1) to give 2-fluoro-3-[2-[1-(trifluoromethyl)cyclobutyl]oxiran-2-yl]pyridine (68 g) as a yellow oil.

LC-MS (Method 1): Rt=0.83 min; m/z=262.0 [M+H]⁺.

¹H-NMR: (400 MHz, CDCl₃): δ [ppm]=8.21-8.19 (m, 1H), 7.86-7.85 (m, 1H), 7.20-7.18 (m, 1H), 3.47-3.38 (m, 1H), 2.98-2.97 (m, 1H), 2.52-2.26 (m, 1H), 2.25-1.91 (m, 5H).

### Intermediate 4

### 3-[1-(trifluoromethyl)cyclobutyl]-1,2-dihydropyrrolo[2,3-b]pyridin-3-ol

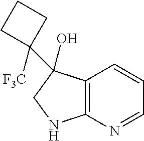 [SMILES: OC1(C2(C(F)(F)F)CCC2)CNc2ncccc21]

To a solution of 2-fluoro-3-[2-[1-(trifluoromethyl)cyclobutyl]oxiran-2-yl]pyridine (Intermediate 3, 68 g, 260.32 mmol) in tetrahydrofuran (120 mL) was added aqueous ammonia (1.5 L, 28% purity) in one portion at room temperature. The mixture was stirred at 100° C. in an autoclave for 72 hours. The reaction mixture was cooled to room temperature and extracted with ethyl acetate (2 L). The organic phase was washed with brine (500 mL), dried over sodium sulfate, filtered and concentrated under reduced pressure to give 3-[1-(trifluoromethyl)cyclobutyl]-1,2-dihydropyrrolo[2,3-b]pyridin-3-ol (69 g, crude) as a yellow oil.

¹H-NMR: (400 MHz, CDCl₃): δ [ppm]=7.89-7.87 (m, 1H), 7.55-7.53 (m, 1H), 6.59-6.54 (m, 1H), 4.62 (brs, 1H), 3.80-3.77 (m, 1H), 3.50-3.47 (m, 1H), 2.61-2.56 (m, 2H), 2.39-2.35 (m, 2H), 2.06-2.04 (m, 2H).

### Intermediate 5

### 3-[1-(trifluoromethyl)cyclobutyl]-1H-pyrrolo[2,3-b]pyridine

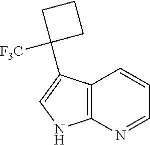 [SMILES: FC(F)(F)C1(c2c[nH]c3ncccc23)CCC1]

To a solution of 3-[1-(trifluoromethyl)cyclobutyl]-1,2-dihydropyrrolo[2,3-b]pyridin-3-ol (Intermediate 4, 69 g, crude) and pyridine (56 mL, 693.81 mmol) in dichloromethane (700 mL) was added thionyl chloride (50 mL, 689.25 mmol) at 0° C. under nitrogen. The resulting solution was stirred at room temperature for 16 hours. Ice-water (200 mL) was added to the solution slowly at 0° C. To the resulting mixture was added sat. aq. sodium hydrogencarbonate drop-wise until no bubbles appeared. The two phases were separated, and the organic phase was washed with brine (200 mL), dried over magnesium sulfate, filtered and concentrated under reduced pressure. The residue was slurried with tert-butyl methyl ether (150 mL) at room temperature for 10 min. The suspension was filtered to yield 3-[1-(trifluoromethyl)cyclobutyl]-1H-pyrrolo[2,3-b]pyridine (41 g) as a light yellow solid.

¹H-NMR: (400 MHz, DMSO-d6): δ [ppm]=11.77 (brs, 1H), 8.24-8.23 (m, 1H), 7.80 (d, 1H), 7.58 (d, 1H), 7.08-7.04 (m, 1H), 2.71-2.66 (m, 2H), 2.57-2.55 (m, 2H), 2.00-1.98 (m, 2H).

### Intermediate 6

### 3-(1-(trifluoromethyl)cyclobutyl)-1H-pyrrolo[2,3-b]pyridine 7-oxide

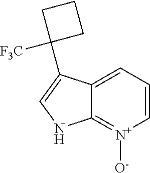 [SMILES: [O-][n+]1cccc2c(C3(C(F)(F)F)CCC3)c[nH]c21]

To a solution of 3-[1-(trifluoromethyl)cyclobutyl]-1H-pyrrolo[2,3-b]pyridine (Intermediate 5, 31 g, 129.05 mmol) in dichloromethane (500 mL) was added mCPBA (40 g, 197.03 mmol, 85% purity) at 0° C. The mixture was stirred at room temperature for 16 hours. To the reaction solution was added sat. aq. sodium sulfate (500 mL) at room temperature. The resulting mixture was stirred at room temperature for 30 min. The two phases were separated, and the organic phase was washed with sat. sodium hydrogencarbonate (300 mL×3), brine (300 mL), dried over magnesium sulfate, filtered and concentrated under reduced pressure to give 3-(1-(trifluoromethyl)cyclobutyl)-1H-pyrrolo[2,3-b]pyridine 7-oxide (48 g, crude) as a yellow solid.

LC-MS (Method 1): Rt=0.72 min; m/z=257.1 [M+H]⁺.

### Intermediate 7

### 4-nitro-3-(1-(trifluoromethyl)cyclobutyl)-1H-pyrrolo[2,3-b]pyridine 7-oxide

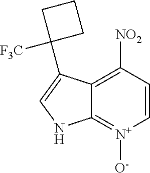 [SMILES: O=[N+]([O-])c1cc[n+]([O-])c2[nH]cc(C3(C(F)(F)F)CCC3)c12]

To a mixture of 3-(1-(trifluoromethyl)cyclobutyl)-1H-pyrrolo[2,3-b]pyridine 7-oxide (Intermediate 6, 40 g, crude) in trifluoroacetic acid (400 mL) was added nitric acid (22 mL, 317.71 mmol, 65% purity) drop-wise at 0° C. The mixture was stirred at room temperature for 16 hours. The reaction mixture was poured into ice-water (500 mL). The resulting mixture was extracted with dichloromethane (500 mL×2). The combined organic phase was washed with brine (300 mL×2), dried over magnesium sulfate, filtered and concentrated by under reduced pressure. The residue was dissolved with dichloromethane (800 mL). The resulting solution was washed with sat. aq. sodium hydrogencarbonate (500 mL), brine (500 mL), dried over magnesium sulfate, filtered and concentrated under reduced pressure to give 30 g of crude product. This crude product was combined with another batch of crude product (20 g) in dichloromethane (500 mL).

The resulting solution was concentrated under reduced pressure. The residue was dried in high vacuum to give 4-nitro-3-(1-(trifluoromethyl)cyclobutyl)-1H-pyrrolo[2,3-b]pyridine 7-oxide (38 g, crude) as a yellow solid.

LC-MS (Method 1): Rt=0.72 min; m/z=302.1 [M+H]⁺.

### Intermediate 8

### 6-chloro-4-nitro-3-[1-(trifluoromethyl)cyclobutyl]-1H-pyrrolo[2,3-b]pyridine

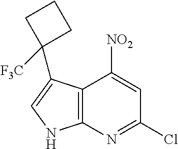 [SMILES: O=[N+]([O-])c1cc(Cl)nc2[nH]cc(C3(C(F)(F)F)CCC3)c12]

To a solution of 4-nitro-3-(1-(trifluoromethyl)cyclobutyl)-1H-pyrrolo[2,3-b]pyridine 7-oxide (Intermediate 7, 28 g, crude) in tetrahydrofuran (300 mL) was added hexamethyldisilazane (22 mL, 104.96 mmol) at 0° C. under nitrogen. Then 2,2,2-trichloroacetyl chloride (22 mL, 197.22 mmol) was added drop-wise to the solution at 0° C. The reaction solution was allowed to warm and stirred at room temperature for 3 hours. The reaction mixture was poured into ice-water (500 mL). The resulting mixture was extracted with ethyl acetate (1 L). The organic phase was washed with sat. aq. sodium hydrogencarbonate (500 mL×3), brine (500 mL), dried over sodium sulfate, filtered and concentrated under reduced pressure to give 40 g of crude product. This crude product was combined with another batch of crude product (15 g) in tert-butyl methyl ether (400 mL). The resulting solution was concentrated under reduced pressure. The residue was slurried with a mixture of petroleum ether and tert-butyl methyl ether (4:1, 300 mL) to give 6-chloro-4-nitro-3-[1-(trifluoromethyl)cyclobutyl]-1H-pyrrolo[2,3-b]pyridine (37 g, crude) as a yellow solid.

LC-MS (Method 1): Rt=1.07 min; m/z=320.0 [M+H]⁺.

¹H-NMR: (400 MHz, CDCl₃): δ [ppm]=10.91 (brs, 1H), 7.50-7.53 (m, 2H), 2.84-2.80 (m, 2H), 2.70-2.68 (m, 2H), 1.97-1.94 (m, 1H), 1.93-1.84 (m, 1H).

### Intermediate 9

### 6-chloro-4-nitro-3-(1-(trifluoromethyl)cyclobutyl)-1-((2-(trimethylsilyl)ethoxy)methyl)-1H-pyrrolo[2,3-b]pyridine

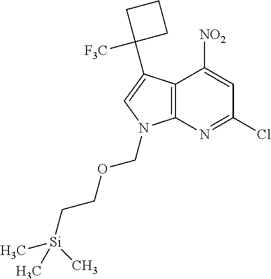 [SMILES: C[Si](C)(C)CCOCn1cc(C2(C(F)(F)F)CCC2)c2c([N+](=O)[O-])cc(Cl)nc21]

To a solution of 6-chloro-4-nitro-3-[1-(trifluoromethyl)cyclobutyl]-1H-pyrrolo[2,3-b]pyridine (Intermediate 8, 22 g, crude) in N,N-dimethylformamide (200 mL) was added N,N-diisopropylethylamine (25.3 mL, 145.25 mmol) at 0° C. The solution was stirred at 0° C. for 10 min. Then 2-(trimethylsilyl)ethoxymethyl chloride (16.50 mL, 93.23 mmol) was added drop-wise to the solution at 0° C. The resulting solution was stirred at 0° C. for 20 min. The mixture was diluted with tert-butyl methyl ether (500 mL), washed with water (100 mL×2), brine (100 mL), dried over sodium sulfate, filtered and concentrated under reduced pressure to give 32 g of crude product. The crude product was purified by silica gel chromatography (petroleum ether:etyl acetate=10:1) to give 6-chloro-4-nitro-3-(1-(trifluoromethyl)cyclobutyl)-1-((2-(trimethylsilyl)ethoxy)methyl)-1H-pyrrolo[2,3-b]pyridine (13 g, 42% yield) as a yellow oil.

¹H-NMR: (400 MHz, CDCl₃): δ [ppm]=7.53-7.52 (m, 2H), 5.69 (s, 2H), 3.61-3.56 (m, 2H), 2.84-2.79 (m, 2H), 2.70-2.67 (m, 2H), 2.29-2.13 (m, 1H), 2.02-1.88 (m, 1H), 0.96-0.92 (m, 2H), −0.03 (s, 9H).

### Intermediate 10

### 2,6-difluoro-4-nitrophenol

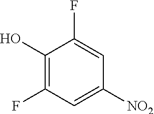 [SMILES: O=[N+]([O-])c1cc(F)c(O)c(F)c1]

To a solution of 1,2,3-trifluoro-5-nitrobenzene (CAS No: 66684-58-0, 100 g, 564.71 mmol) in t-butanol (1 L) was added potassium hydroxide (160 g, 2.85 mol). The mixture was stirred at 80° C. under nitrogen for 16 hours. The mixture was poured into ice-water (2 L), and diluted with ethyl acetate (1.5 L). Concentrated hydrochloric acid (12 M) was added to the mixture until pH 1. The two layers were separated and the aqueous layer was extracted with ethyl acetate (1.5 L). The organic layer was washed with brine (1.5 L), dried over sodium sulfate, filtered and concentrated under reduced pressure to afford 2,6-difluoro-4-nitro-phenol (100 g, crude) as a brown solid.

¹H-NMR: (400 MHz, DMSO-d6): δ [ppm]=8.06-7.96 (m, 2H).

### Intermediate 11

### 4-amino-2,6-difluorophenol

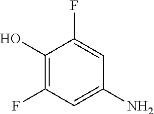

To a solution of 2,6-difluoro-4-nitro-phenol (Intermediate 10, 90 g, crude) in methanol (900 mL) was added palladium on charcoal (13.5 g, 10% purity—wet, contains 50% of water) at room temperature under nitrogen. The suspension was put under vacuum and purged with hydrogen 3 times. The mixture was stirred under a hydrogen atmosphere (15 psi). The reaction was exothermal, and the mixture was stirred at 45° C. for 3 hours and then gradually cooled to room temperature and stirred for an additional 14 hours. The mixture was filtered through a pad of celite, and the filter cake washed with MeOH (300 mL). The filtrate was concentrated by under reduced pressure. The resulting residue was put under nitrogen, suspended in dioxane (150 mL), and cooled down to 0° C. Hydrochloric acid/dioxane (4 M, 200 mL) was slowly added. The suspension was stirred at room temperature for 15 min and filtered to afford 4-amino-2,6-difluorophenol (85 g, crude, hydrochloric acid salt) as an off-white solid.

¹H-NMR: (400 MHz, DMSO-d6): δ [ppm]=9.95 (brs, 2H), 7.09-7.01 (m, 2H).

### Intermediate 12

### 4-((6-chloro-3-(1-(trifluoromethyl)cyclobutyl)-1-((2-(trimethylsilyl)ethoxy)methyl)-1H-pyrrolo[2,3-b]pyridin-4-yl)oxy)-3,5-difluoroaniline

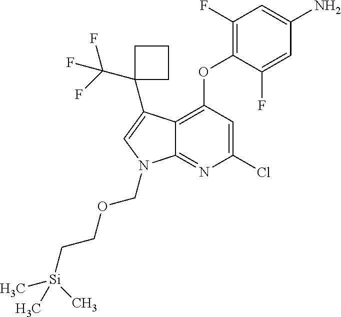 [SMILES: C[Si](C)(C)CCOCn1cc(C2(C(F)(F)F)CCC2)c2c(Oc3c(F)cc(N)cc3F)cc(Cl)nc21]

To a solution of 6-chloro-4-nitro-3-(1-(trifluoromethyl)cyclobutyl)-1-((2-(trimethylsilyl)ethoxy)methyl)-1H-pyrrolo[2,3-b]pyridine (Intermediate 9, 15.4 g, 34.23 mmol) and 4-amino-2,6-difluoro-phenol (Intermediate 11, 10 g, crude, hydrochloric acid salt) in dimethyl sulfoxide (150 mL) was added potassium carbonate (19 g, 137.48 mmol) at room temperature under nitrogen. The mixture was stirred at 50° C. for 16 hours. The reaction mixture was cooled to room temperature and filtered through a pad of celite. Ethyl acetate (500 mL) and water (100 mL) were added to the filtrate, and the two phases were separated. The organic phase was washed with water (200 mL×2), brine (100 mL), dried over sodium sulfate, filtered and concentrated under reduced pressure. The residue was purified by silica gel chromatography (petroleum ether:ethyl acetate=4:1) to give 4-((6-chloro-3-(1-(trifluoromethyl)cyclobutyl)-1-((2-(trimethylsilyl)ethoxy)methyl)-1H-pyrrolo[2,3-b]pyridin-4-yl)oxy)-3,5-difluoroaniline (16 g, 85% yield) as a yellow solid. ¹H-NMR: (400 MHz, CDCl₃): δ [ppm]=7.18 (s, 1H), 6.34-6.32 (m, 3H), 5.61 (s, 2H), 3.92 (brs, 2H), 3.61-3.57 (m, 2H), 2.81-2.76 (m, 2H), 2.68-2.66 (m, 2H), 2.16-2.13 (m, 1H), 2.00-1.95 (m, 1H), 0.94-0.90 (m, 2H), −0.04 (s, 9H).

### Intermediate 13

### 3,5-difluoro-4-((3-(1-(trifluoromethyl)cyclobutyl)-1-((2-(trimethylsilyl)ethoxy)methyl)-1H-pyrrolo[2,3-b]pyridin-4-yl)oxy)aniline

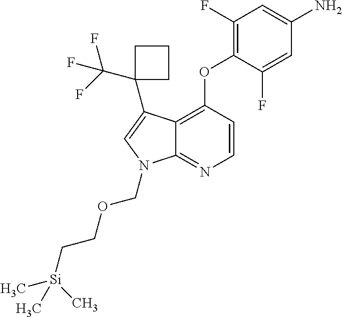 [SMILES: C[Si](C)(C)CCOCn1cc(C2(C(F)(F)F)CCC2)c2c(Oc3c(F)cc(N)cc3F)ccnc21]

To a solution of 4-((6-chloro-3-(1-(trifluoromethyl) cyclobutyl)-1-((2-(trimethylsilyl)ethoxy)methyl)-1H-pyrrolo[2,3-b]pyridin-4-yl)oxy)-3,5-difluoroaniline (Intermediate 12, 16 g, 29.20 mmol) in tetrahydrofuran (150 mL) was added triethylamine (6.2 mL, 44.54 mmol) and palladium on charcoal (3 g, 10% purity—wet, contains 50% of water) under nitrogen at room temperature. The suspension was put under vacuum and purged with hydrogen several times. The mixture was stirred under hydrogen atmosphere (15 psi) at 45° C. for 12 hours. The mixture was filtered through a pad of celite, and the filtrate concentrated under reduced pressure. The residue was re-dissolved in tetrahydrofuran (150 mL), and subjected again to the reaction conditions. This operation had to be repeated once more (a total of 3 times) to achieve reaction completion. The reaction mixture was cooled to room temperature, then filtered through a pad of celite. The filtrate was concentrated under reduced pressure. The residue was purified by silica gel chromatography (petroleum ether:ethyl acetate=5:1 to 3:1) and the resulting residue dissolved in 20 mL of acetonitrile. Upon addition of water (100 mL) a white solid precipitated. The resulting mixture was lyophilized to give 3,5-difluoro-4-((3-(1-(trifluoromethyl)cyclobutyl)-1-((2-(trimethylsilyl)ethoxy)methyl)-1H-pyrrolo[2,3-b]pyridin-4-yl)oxy)aniline (12.5 g, 83% yield) as a white solid.

LC-MS (Method 2): Rt=2.57 min; m/z=514.2 [M+H]⁺.

¹H-NMR: (400 MHz, CDCl₃): δ [ppm]=8.13 (s, 1H), 7.21 (s, 1H), 6.35-6.30 (m, 3H), 5.67 (s, 2H), 3.87 (brs, 2H), 3.62-3.57 (m, 2H), 2.83-2.80 (m, 2H), 2.78-2.70 (m, 2H), 2.16-2.14 (m, 1H), 1.99-1.97 (m, 1H), 0.94-0.90 (m, 2H), −0.04 (s, 9H).

### Intermediate 14

### O-phenyl {3,5-difluoro-4-[(3-[1-(trifluoromethyl)cyclobutyl]-1-{[2-(trimethylsilyl)ethoxy]methyl}-1H-pyrrolo[2,3-b]pyridin-4-yl)oxy]phenyl}carbamothioate

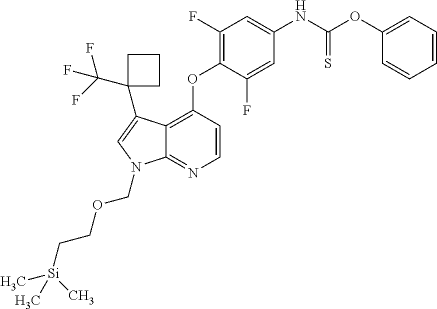 [SMILES: C[Si](C)(C)CCOCn1cc(C2(C(F)(F)F)CCC2)c2c(Oc3c(F)cc(NC(=S)Oc4ccccc4)cc3F)ccnc21]

3,5-difluoro-4-[(3-[1-(trifluoromethyl)cyclobutyl]-1-{[2-(trimethylsilyl)ethoxy]methyl}-1H-pyrrolo[2,3-b]pyridin-4-yl)oxy]aniline (Intermediate 13, 400 mg, 779 μmol) was dissolved in a mixture of THF/pyridine (6.2 mL:2.8 mL) and cooled down to 0° C. O-phenyl carbonochloridothioate (120 μl, 860 μmol) was added dropwise, and the resulting reaction stirred for 1 h at 0° C. The solvent was removed under vacuum and the product used in the next step with no further purification.

LC-MS (Method 3): Rt=1.77 min; MS (ESIpos): m/z=651 [M+H]⁺

### Intermediate 15

### N-{3,5-difluoro-4-[(3-[1-(trifluoromethyl)cyclobutyl]-1-{[2-(trimethylsilyl)ethoxy]methyl}-1H-pyrrolo[2,3-b]pyridin-4-yl)oxy]phenyl}-N′-{[1-(hydroxymethyl)cyclobutyl]methyl}thiourea

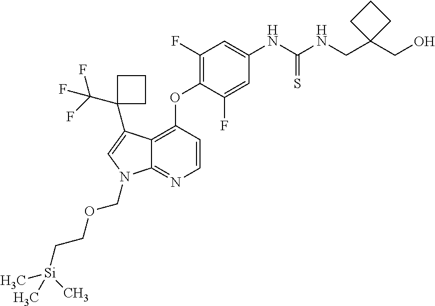 [SMILES: C[Si](C)(C)CCOCn1cc(C2(C(F)(F)F)CCC2)c2c(Oc3c(F)cc(NC(=S)NCC4(CO)CCC4)cc3F)ccnc21]

O-phenyl {3,5-difluoro-4-[(3-[1-(trifluoromethyl)cyclobutyl]-1-{[2-(trimethylsilyl)ethoxy]methyl}-1H-pyrrolo[2,3-b]pyridin-4-yl)oxy]phenyl}carbamothioate (Intermediate 14, 253 mg, 390 μmol) and [1-(aminomethyl)cyclobutyl]methanol (89.7 mg, 779 μmol, CAS No. [2041-56-7]) were dissolved in N,N-dimethylformamide (2.3 mL) and stirred for 2 h at 60° C. To the solution was added brine, and the mixture was extracted with ethyl acetate. The organic phase was dried over sodium sulfate, filtered and the solvent removed under vacuum. The crude was purified by silica gel chromatography to yield the title compound (239 mg, 82% yield).

LC-MS (Method 4): Rt=1.63 min; MS (ESIpos): m/z=671 [M+H]⁺

¹H-NMR: (400 MHz, CDCl₃): δ [ppm]=10.00 (s, 1H), 8.16 (d, 1H), 8.00 (m, 1H), 7.77 (s, 1H), 7.57 (m, 2H), 6.39 (m, 1H), 5.64 (s, 2H), 4.87 (br t, 1H), 3.67 (br d, 2H), 3.55 (t, 2H), 3.45 (br d, 2H), 2.68 (m, 4H), 2.02-1.74 (m, 8H), 8.31 (t, 2H), −0.11 (s, 9H).

### Intermediate 16

### N-[3,5-difluoro-4-({3-[1-(trifluoromethyl)cyclobutyl]-1H-pyrrolo[2,3-b]pyridin-4-yl}oxy)phenyl]-N′-{[1-(hydroxymethyl)cyclobutyl]methyl}thiourea

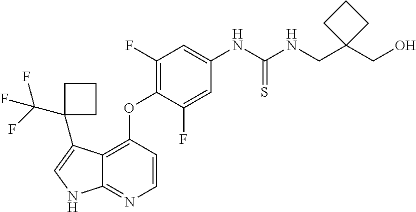 [SMILES: OCC1(CNC(=S)Nc2cc(F)c(Oc3ccnc4[nH]cc(C5(C(F)(F)F)CCC5)c34)c(F)c2)CCC1]

N-{3,5-difluoro-4-[(3-[1-(trifluoromethyl)cyclobutyl]-1-{[2-(trimethylsilyl)ethoxy]methyl}-1H-pyrrolo[2,3-b]pyridin-4-yl)oxy]phenyl}-N′-{[1-(hydroxymethyl)cyclobutyl]methyl}thiourea (Intermediate 15, 236 mg, 352 μmol) was dissolved in dichloromethane (11 mL) under argon, and trifluoroacetic acid (1.2 mL, 15 mmol) was added dropwise. The mixture was stirred at room temperature overnight. Acetonitrile (11 mL) and aqueous ammonia (33%, 6 mL) were added, and the solution was stirred for 1 h. The solvent was removed under vacuum, and the residue washed with water and dried to yield the title compound as a white solid (181 mg, 86% yield).

LC-MS (Method 3): Rt=1.27 min; MS (ESIpos): m/z=541 [M+H]⁺

¹H-NMR (400 MHz, DMSO-d6) δ [ppm]: 11.98 (d, 1H), 9.99 (s, 1H), 8.08 (d, 1H), 7.99 (m, 1H), 7.57 (br d, 2H), 7.53 (d, 1H), 6.28 (m, 1H), 4.78 (br t, 1H), 3.66 (br d, 2H), 3.44 (d, 2H), 2.67 (m, 4H), 2.02-1.74 (m, 8H).

### Intermediate 17

### N-{3,5-difluoro-4-[(3-[1-(trifluoromethyl)cyclobutyl]-1-{[2-(trimethylsilyl)ethoxy]methyl}-1H-pyrrolo[2,3-b]pyridin-4-yl)oxy]phenyl}-N′-{[4-(hydroxymethyl)oxan-4-yl]methyl}thiourea

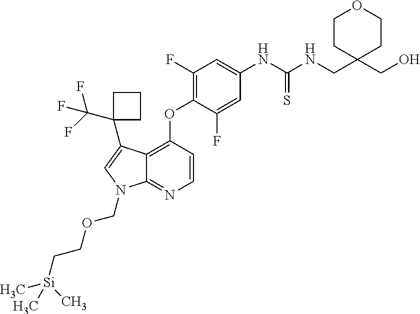 [SMILES: C[Si](C)(C)CCOCn1cc(C2(C(F)(F)F)CCC2)c2c(Oc3c(F)cc(NC(=S)NCC4(CO)CCOCC4)cc3F)ccnc21]

O-phenyl {3,5-difluoro-4-[(3-[1-(trifluoromethyl)cyclobutyl]-1-{[2-(trimethylsilyl)ethoxy]methyl}-1H-pyrrolo[2,3-b]pyridin-4-yl)oxy]phenyl}carbamothioate (Intermediate 14, 253 mg, 390 μmol) and [4-(aminomethyl)oxan-4-yl]methanol (113 mg, 779 μmol, CAS No. [959238-22-3]) were dissolved in N,N-dimethylformamide (2.3 mL) and stirred for 2 h at 60° C. To the solution was added brine, and the mixture was extracted with ethyl acetate. The organic phase was dried over sodium sulfate, filtered and the solvent removed under vacuum. The crude was purified by silica gel chromatography to yield the title compound (253 mg, 91% yield).

LC-MS (Method 4): Rₜ=1.56 min; MS (ESIpos): m/z=701 [M+H]⁺

¹H-NMR (400 MHz, DMSO-d6) δ [ppm]: 10.09 (s, 1H), 8.16 (d, 1H), 8.03 (m, 1H), 7.75 (s, 1H), 7.58 (m, 2H), 6.34 (d, 1H), 5.63 (s, 2H), 4.94 (br t, 1H), 3.70 (br d, 2H), 3.62-3.53 (m, 6H), 3.39 (d, 2H), 2.667 (m, 4H), 2.07-1.91 (m, 2H), 1.41 (m, 4H), 0.80 (t, 2H), −0.11 (s, 9H).

### Intermediate 18

### N-[3,5-difluoro-4-({3-[1-(trifluoromethyl)cyclobutyl]-1H-pyrrolo[2,3-b]pyridin-4-yl}oxy)phenyl]-N′-{[4-(hydroxymethyl)oxan-4-yl]methyl}thiourea

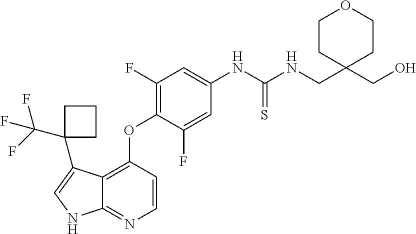 [SMILES: OCC1(CNC(=S)Nc2cc(F)c(Oc3ccnc4[nH]cc(C5(C(F)(F)F)CCC5)c34)c(F)c2)CCOCC1]

N-{3,5-difluoro-4-[(3-[1-(trifluoromethyl)cyclobutyl]-1-{[2-(trimethylsilyl)ethoxy]methyl}-1H-pyrrolo[2,3-b]pyridin-4-yl)oxy]phenyl}-N′-{[4-(hydroxymethyl)oxan-4-yl]methyl}thiourea (Intermediate 17, 250 mg, 357 μmol) was dissolved in dichloromethane (11 mL) under argon, and trifluoroacetic acid (1.2 mL, 15 mmol) was added dropwise. The mixture was stirred at room temperature overnight. Acetonitrile (11 mL) and aqueous ammonia (33%, 6 mL) were added, and the solution was stirred for 1 h. The solvent was removed under vacuum, and the residue washed with water and dried to yield the title compound as a white solid (181 mg, 84% yield).

LC-MS (Method 3): Rₜ=1.16 min; MS (ESIpos): m/z=571 [M+H]⁺

¹H-NMR (400 MHz, DMSO-d6) δ [ppm]: 11.99 (br d, 1H), 10.08 (br s, 1H), 8.08 (d, 1H), 8.01 (m, 1H), 7.58 (d, 2H), 7.53 (d, 1H), 6.28 (d, 1H), 4.93 (br t, 1H), 3.70 (br d, 2H), 3.59 (m, 4H), 3.39 (br d, 2H), 2.67 (br t, 4H), 2.05-1.91 (m, 2H), 1.39 (br t, 4H).

### Intermediate 19

### N-{3,5-difluoro-4-[(3-[1-(trifluoromethyl)cyclobutyl]-1-{[2-(trimethylsilyl)ethoxy]methyl}-1H-pyrrolo[2,3-b]pyridin-4-yl)oxy]phenyl}-N′-{[1-(hydroxymethyl)cyclopropyl]methyl}thiourea

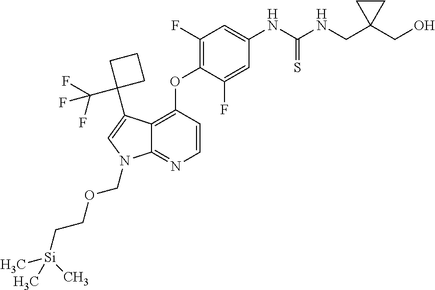 [SMILES: C[Si](C)(C)CCOCn1cc(C2(C(F)(F)F)CCC2)c2c(Oc3c(F)cc(NC(=S)NCC4(CO)CC4)cc3F)ccnc21]

O-phenyl {3,5-difluoro-4-[(3-[1-(trifluoromethyl)cyclobutyl]-1-{[2-(trimethylsilyl)ethoxy]methyl}-1H-pyrrolo[2,3-b]pyridin-4-yl)oxy]phenyl}carbamothioate (Intermediate 14, 190 mg, 292 μmol) and 1-(aminomethyl)cyclopropyl]methanol (59.2 mg, 585 μmol, CAS No. [45434-02-4]) were dissolved in N,N-dimethylformamide (1 mL) and stirred overnight at 60° C. The solvent was removed under vacuum, and the crude was purified by silica gel chromatography to yield the title compound (228 mg, quantitative).

LC-MS (Method 4): Rₜ=1.59 min; MS (ESIpos): m/z=657 [M+H]⁺

¹H-NMR (400 MHz, DMSO-d6) δ [ppm]: 10.00 (s, 1H), 8.16 (d, 1H), 8.10 (m, 1H), 7.77 (s, 1H), 7.59 (m, 2H), 6.38 (m, 1H), 5.62 (s, 2H), 4.78 (br t, 1H), 3.57-3.48 (m, 4H), 3.35 (m, 2H), 2.71-2.62 (m, 4H), 2.08-1.90 (m, 2H), 0.80 (t, 2H), 0.50 (m, 2H), 0.44 (m, 2H), −0.11 (s, 9H).

### Intermediate 20

### N-{3,5-difluoro-4-[(3-[1-(trifluoromethyl)cyclobutyl]-1-{[2-(trimethylsilyl)ethoxy]methyl}-1H-pyrrolo[2,3-b]pyridin-4-yl)oxy]phenyl}-5-oxa-7-azaspiro[2.5]oct-6-en-6-amine

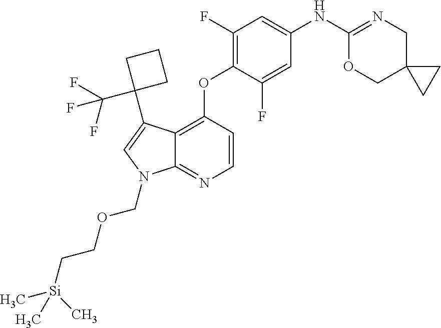 [SMILES: C[Si](C)(C)CCOCn1cc(C2(C(F)(F)F)CCC2)c2c(Oc3c(F)cc(NC4=NCC5(CC5)CO4)cc3F)ccnc21]

N-{3,5-difluoro-4-[(3-[1-(trifluoromethyl)cyclobutyl]-1-{[2-(trimethylsilyl)ethoxy]methyl}-1H-pyrrolo[2,3-b]pyridin-4-yl)oxy]phenyl}-N′-{[1-(hydroxymethyl)cyclopropyl]methyl}thiourea (Intermediate 19, 230 mg, 350 μmol), 1-(3-Dimethylaminopropyl)-3-ethylcarbodiimidhydrochloride (134 mg, 700 μM), and triethylamine (150 μl, 1.1 mmol) were stirred in acetonitrile (3 mL) at 40° C. overnight. The solvent was removed under vacuum and the crude filtered through silica to yield the title compound (119 mg, 55% yield), which was used in the following reaction with no further purification.

LC-MS (Method 4): Rₜ=1.68 min; MS (ESIpos): m/z=623 [M+H]⁺

### Intermediate 21

### N-{3,5-difluoro-4-[(3-[1-(trifluoromethyl)cyclobutyl]-1-{[2-(trimethylsilyl)ethoxy]methyl}-1H-pyrrolo[2,3-b]pyridin-4-yl)oxy]phenyl}-N′-(3-hydroxy-2,2-dimethylpropyl)thiourea

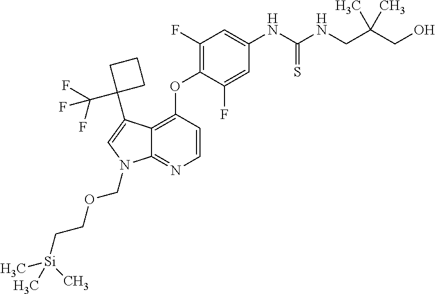 [SMILES: CC(C)(CO)CNC(=S)Nc1cc(F)c(Oc2ccnc3c2c(C2(C(F)(F)F)CCC2)cn3COCC[Si](C)(C)C)c(F)c1]

O-phenyl {3,5-difluoro-4-[(3-[1-(trifluoromethyl)cyclobutyl]-1-{[2-(trimethylsilyl)ethoxy]methyl}-1H-pyrrolo[2,3-b]pyridin-4-yl)oxy]phenyl}carbamothioate (Intermediate 14, 190 mg, 292 μmol) and 3-amino-2,2-dimethylpropan-1-ol (60.3 mg, 585 μmol, CAS No. [141-43-5]) were dissolved in N,N-dimethylformamide (1 mL) and stirred overnight at 60° C. The solvent was removed under vacuum, and the crude was filtered through silica to yield the title compound (230 mg, quantitative), that was used in the following reaction with no further purification.

LC-MS (Method 4): Rₜ=1.62 min; MS (ESIpos): m/z=659 [M+H]⁺

### Intermediate 22

### N-{3,5-difluoro-4-[(3-[1-(trifluoromethyl)cyclobutyl]-1-{[2-(trimethylsilyl)ethoxy]methyl}-1H-pyrrolo[2,3-b]pyridin-4-yl)oxy]phenyl}-5,5-dimethyl-5,6-dihydro-4H-1,3-oxazin-2-amine

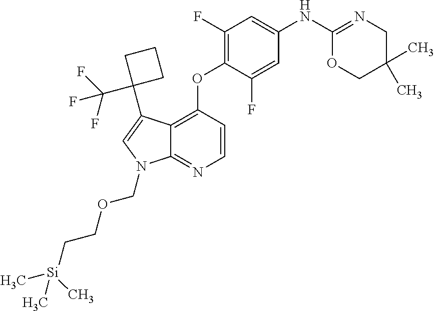

N-{3,5-difluoro-4-[(3-[1-(trifluoromethyl)cyclobutyl]-1-{[2-(trimethylsilyl)ethoxy]methyl}-1H-pyrrolo[2,3-b]pyridin-4-yl)oxy]phenyl}-N′-{[1-(hydroxymethyl)cyclopropyl]methyl}thiourea (Intermediate 21,230 mg, 349 μmol), 1-(3-Dimethylaminopropyl)-3-ethylcarbodiimidhydrochloride (134 mg, 700 μmol), and triethylamine (150 μl, 1.1 mmol) were stirred in acetonitrile (3 mL) at 40° C. overnight. The solvent was removed under vacuum and the crude filtered through silica to yield the title compound (124 mg, 57% yield), which was used in the following reaction with no further purification.

LC-MS (Method 4): Rₜ=1.70 min; MS (ESIpos): m/z=625 [M+H]⁺

### Intermediate 23

### (2-fluoropyridin-3-yl)[1-(trifluoromethyl)cyclopropyl]methanone

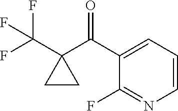 [SMILES: O=C(c1cccnc1F)C1(C(F)(F)F)CC1]

To a solution of 2-fluoropyridine (1.9 g, 19.6 mmol) in THF (40 mL) was added LDA (1 M, 25 mL, freshly prepared) drop-wise at −78° C. under N₂. The mixture was stirred at −78° C. for 1 hour. Then N-methoxy-N-methyl-1 (trifluoromethyl)cyclopropanecarboxamide (3.8 g, 19.3 mmol, prepared as described in Org. Process Res. Dev., 2009, 13 (3), pp 576-580) was added. The mixture was warmed to 15° C. and stirred for 1 hour. TLC (Petroleum ether: Ethyl acetate=5:1) indicated the reaction completed. The mixture was quenched by adding a saturated aqueous solution of ammonium chloride (50 mL). The mixture was extracted with ethyl acetate (100 mL×2). The combined organic phase was washed with brine (100 mL×2), dried over anhydrous sodium sulfate, filtered and concentrated by rotary evaporator in vacuum. The residue was purified by chromatography on silica gel (Petroleum ether: Ethyl acetate=100:1) to give (2-fluoropyridin-3-yl)(1-(trifluoromethyl)cyclopropyl)methanone (2.4 g, 53% yield) as a yellow oil.

¹H NMR (CDCl₃, 400 MHz): 5=1.65-1.54 (m, 4H), 7.31 (t, 1H), 7.87 (t, 1H), 8.38-8.37 (m, 1H).

### Intermediate 24

### 2-fluoro-3-{2-[1-(trifluoromethyl)cyclopropyl]oxiran-2-yl}pyridine

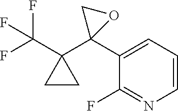 [SMILES: Fc1ncccc1C1(C2(C(F)(F)F)CC2)CO1]

NaH (900 mg, 22.50 mmol, 60% purity) was added into DMSO (40 mL) at 15° C. in one portion. The mixture was heated to 65° C. for 1 hour. Then the mixture was cooled to 15° C. and trimethylsulfoxonium iodide (4.80 g, 21.81 mmol) was added. The mixture was stirred at 15° C. for 1 hour. Then (2-fluoropyridin-3-yl)[1-(trifluoromethyl)cyclopropyl]methanone (2.4 g, 10.3 mmol, intermediate 23) was added. The mixture was stirred at 15° C. for further 13 hours. LC-MS indicated the reaction completed. The reaction mixture was quenched by water (100 mL) slowly. The suspension was extracted with ethyl acetate (100 mL×2). The combined organic phase was washed with brine (100 mL×2), dried over anhydrous sodium sulfate, filtered and concentrated by rotary evaporator in vacuum to give 2-fluoro-3-(2-(1-(trifluoromethyl)cyclopropyl)oxiran-2-yl)pyridine, which was used without further purification.

LC-MS (Method 5): Rₜ=0.82 min; MS (ESIpos): m/z=248 [M+H]⁺

### Intermediate 25

### 3-[1-(trifluoromethyl)cyclopropyl]-2,3-dihydro-1H-pyrrolo[2,3-b]pyridin-3-ol

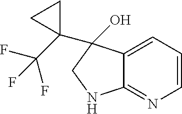 [SMILES: OC1(C2(C(F)(F)F)CC2)CNc2ncccc21]

To a solution of 2-fluoro-3-{2-[1-(trifluoromethyl)cyclopropyl]oxiran-2-yl}pyridine (2.4 g, 9.7 mmol, intermediate 24) in THF (12 mL) was added aq. ammonia solution (50 mL, 364 mmol, 28% purity) at 10° C. The mixture was stirred at 60° C. for 32 hours. LC-MS indicated the reaction completed. The mixture was poured into water (50 mL). The suspension was extracted with ethyl acetate (50 mL×3). The combined organic phase was washed with brine (50 mL×2), dried over anhydrous sodium sulfate, filtered and concentrated by rotary evaporator in vacuum. The residue was purified by prep-HPLC (column: Phenomenex Gemini C18 250*50 mm*10 um; mobile phase: [water (0.05% ammonia hydroxide v/v)-ACN]; B %: 20%-45%, 26 MIN; 78% min) to get a solution, which was concentrated to 100 mL at 30° C. by rotary evaporator in vacuum. The formed solid was collected by filtration and dried in vacuum to give the first batch of 3-(1-(trifluoromethyl)cyclopropyl)-2,3-dihydro-1H-pyrrolo[2,3-b]pyridin-3-ol (1.0 g, 42% yield) as a white solid. The filtrate was lyophilized to give the second batch of 3-(1-(trifluoromethyl)cyclopropyl)-2,3-dihydro-1H-pyrrolo[2,3-b]pyridin-3-ol (200 mg, 8% yield) as a white solid.

¹H NMR (DMSO-de, 400 MHz): 5=0.92-0.88 (m, 2H), 1.09-1.05 (m, 2H), 3.35 (d, 1H), 3.73 (d, 1H), 5.68 (s, 1H), 6.50 (dd, 1H), 6.53 (s, 1H), 7.40 (d, 1H), 7.85 (dd, 1H).

¹⁹F NMR (DMSO-de, 400 MHz): δ=−62

LC-MS (Method 6): Rₜ=0.67 min; MS (ESIpos): m/z=245 [M+H]⁺

### Intermediate 26

### 3-[1-(trifluoromethyl)cyclopropyl]-1H-pyrrolo[2,3-b]pyridine

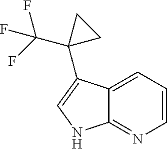 [SMILES: FC(F)(F)C1(c2c[nH]c3ncccc23)CC1]

To a mixture of 3-[1-(trifluoromethyl)cyclopropyl]-2,3-dihydro-1H-pyrrolo[2,3-b]pyridin-3-ol (45 g, 158 mmol, 86% purity, intermediate 25) and pyridine (25 mL, 310 mmol) in dichloromethane (500 mL) was added thionyl chloride (22 mL, 303 mmol) drop-wise at 0° C. under a nitrogene atmosphere. The mixture was stirred at 15° C. for 12 hours. The mixture was poured into ice-water (500 mL) and neutralized to pH=5-6 with 10% aqueous sodium hydroxide. The aqueous phase was extracted with dichloromethane (300 mL×2). The combined organic phase was washed with brine (300 mL×2), dried over sodium sulfate, filtered and concentrated by rotary evaporator in vacuum. The residue was purified by silica gel chromatography (1000 mesh silica gel, petrol ether: ethyl acetate=10:1 to 1:1) to give the title compound (35 g, 98% yield) as a yellow solid.

¹H NMR (400 MHz, DMSO-d₆): 5=1.17-1.14 (m, 2H), 1.39-1.36 (m, 2H), 7.35-7.32 (m, 1H), 7.74 (d, 1H), 9.30 (d, 1H), 8.39-8.37 (m, 1H), 12.41 (s, 1H).

### Intermediate 27

### 3-[1-(trifluoromethyl)cyclopropyl]-1H-pyrrolo[2,3-b]pyridine 7-oxide

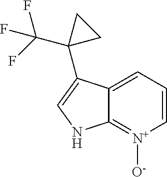 [SMILES: [O-][n+]1cccc2c(C3(C(F)(F)F)CC3)c[nH]c21]

To a solution of 3-[1-(trifluoromethyl)cyclopropyl]-1H-pyrrolo[2,3-b]pyridine (35 g, 155 mmol, intermediate 26) in dichloromethane (350 mL) was added m-chloroperoxybenzoic acid (47 g, 232 mmol, 85% purity) in portions at 0° C. The mixture was stirred at 15° C. for 12 hours. The mixture was filtered, and the filtrate was washed with saturated sodium thiosulfate solution (300 mL×2), brine (300 mL×2), dried over sodium sulfate, filtered and concentrated by rotary evaporator in vacuum. The residue was suspended in methyl tert-butylether (50 mL) and stirred for 30 min. The suspension was filtered, and the cake was washed with methyl tert-butylether (20 mL×2) and dried by in vacuum to give the desired tile compound which was used without further purification.

¹H NMR (400 MHz, DMSO-d₆): δ=1.13 (m, 2H), 1.37-1.34 (m, 2H), 7.15-7.12 (m, 1H), 7.59 (s, 1H), 7.64 (d, 1H), 8.17 (d, 1H), 12.62 (s, 1H).

### Intermediate 28

### 4-nitro-3-[1-(trifluoromethyl)cyclopropyl]-1H-pyrrolo[2,3-b]pyridine 7-oxide

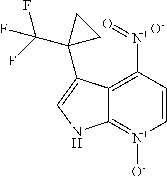

To a solution of 3-[1-(trifluoromethyl)cyclopropyl]-1H-pyrrolo[2,3-b]pyridine 7-oxide (37 g, crude, intermediate 27) in trifluoroacidic acid (400 mL) was added nitric acid (30 g, 309 mmol, 65% purity) drop-wise at 0° C. The mixture was warmed to 15° C. and stirred for 14 hours. Then additional nitric acid (14 g, 222 mmol, 65% purity) was added at 0° C., the mixture was stirred at 15° C. for another 14 hours. The mixture was poured into ice-water (800 mL) and stirred for 10 min. The aqueous phase was extracted with dichloromethane (300 mL×3). The combined organic phase was washed with brine (300 mL×2), dried over sodium sulfate, filtered and concentrated by rotary evaporator in vacuum to give the desired title compound which was used without further purification.

¹H NMR (400 MHz, DMSO-d₆): δ=1.42-1.37 (m, 4H), 7.95-7.90 (m, 2H), 8.35 (d, 1H), 13.49 (s, 1H).

### Intermediate 29

### 6-chloro-4-nitro-3-[1-(trifluoromethyl)cyclopropyl]-1H-pyrrolo[2,3-b]pyridine

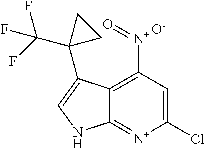

To a solution of 4-nitro-3-[1-(trifluoromethyl)cyclopropyl]-1H-pyrrolo[2,3-b]pyridine 7-oxide (60 g, crude, intermediate 28) in THF (600 mL) was added hexamethyidisilazane (25 mL, 119 mmol) in one portion at 0° C. under nitrogene atmosphere. Then 2,2,2-trichloroacetyl chloride (30 mL, 269 mmol) was added drop-wise. The mixture was warmed to 15° C. and stirred for 12 hours. The mixture was poured into ice-water (1 L) and stirred for 30 min. The aqueous phase was extracted with ethyl acetate (500 mL×2). The combined organic phase was washed with a saturated aqueous solution of sodium bicarbonate (500 mL×2) and brine (500 mL×2), dried over sodium sulfate, filtered and concentrated by rotary evaporator in vacuum. The residue was purified by silica gel chromatography (100-200 mesh silica gel, petrol ether: ethyl acetate=100:1 to 10:1) to give the desired title compound (35 g, 57% purity) as a yellow solid.

¹H NMR (400 MHz, DMSO-d₆): δ=1.39-1.30 (m, 4H), 7.88 (s, 1H), 8.09 (d, 1H).

### Intermediate 30

### 6-chloro-4-nitro-3-[1-(trifluoromethyl)cyclopropyl]-1-{[2-(trimethylsilyl)ethoxy]methyl}-1H-pyrrolo[2,3-b]pyridine

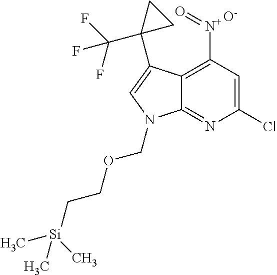

To a solution of 6-chloro-4-nitro-3-[1-(trifluoromethyl)cyclopropyl]-1H-pyrrolo[2,3-b]pyridine (35 g, 65 mmol, 57% purity, intermediate 29) in DMF (350 mL) was added N,N-diisopropyl-ethylamine (24 mL, 138 mmol) at 15° C. The mixture was stirred at 15° C. for 10 min, then 2-(trimethylsilyl)ethoxymethyl chloride (15 mL, 85 mmol) was added. The mixture was stirred at 15° C. for 20 min. The mixture was poured into ice-water (1 L). The aqueous phase was extracted with ethyl acetate (500 mL×2). The combined organic phase was washed with brine (500 mL×2), dried over sodium sulfate, filtered and concentrated by rotary evaporator in vacuum. The residue was purified by silica gel chromatography (100-200 mesh silica gel, petrol ether to petrol ether: ethyl acetate=50:1) to give the desired title compound (25 g, 46.6% yield, 53% purity) as a yellow oil.

¹H NMR (400 MHz, DMSO-d₆): δ=0.12 (s, 9H), 0.86-0.79 (m, 2H), 1.35-1.29 (m, 2H), 1.43 (m, 2H), 3.55 (d, 2H), 5.64 (s, 2H), 7.98 (s, 1H), 8.31 (s, 1H).

### Intermediate 31

### 4-[(6-chloro-3-[1-(trifluoromethyl)cyclopropyl]-1-{[2-(trimethylsilyl)ethoxy]methyl}-1H-pyrrolo[2,3-b]pyridin-4-yl)oxy]-3,5-difluoroaniline

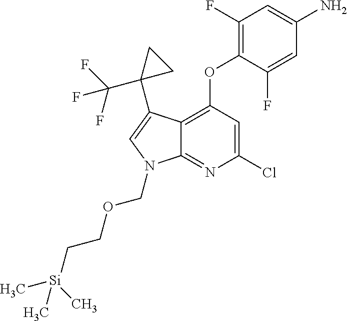 [SMILES: C[Si](C)(C)CCOCn1cc(C2(C(F)(F)F)CC2)c2c(Oc3c(F)cc(N)cc3F)cc(Cl)nc21]

6-chloro-4-nitro-3-[1-(trifluoromethyl)cyclopropyl]-1-{[2-(trimethylsilyl)ethoxy]methyl}-1H-pyrrolo[2,3-b]pyridine (20 g, 24 mmol, 53% purity, intermediate 30) and 4-amino-2,6-difluoro-phenol (5.29 g, 36.5 mmol, intermediate 11) in DMSO (200 mL) was added potassium carbonate (10.07 g, 72.86 mmol) at 15° C. under a nitrogene atmosphere. The mixture was heated to 50° C. and stirred for 2 hours. After cooling to room temperature, the reaction mixture was combined with another second identical reaction mixture using 5 g of 6-chloro-4-nitro-3-(1-(trifluoromethyl)cyclopropyl)-1-((2-(trimethylsilyl)ethoxy)methyl)-1H-pyrrolo[2,3-b]pyridine (intermediate 30). The combined reaction mixtures were poured into ice-water (500 mL). The aqueous phase was extracted with ethyl acetate (500 mL×3). The combined organic phase was washed with brine (500 mL×2), dried over sodium sulfate, filtered and concentrated. The residue was purified by silica gel chromatography (100-200 mesh silica gel, petrol ether: ethyl acetate=30:1 to 10:1) to give the desired title compound (9 g, 86% purity) as a yellow solid. Meanwhile, 6-chloro-4-nitro-3-[1-(trifluoromethyl)cyclopropyl]-1-{[2-(trimethylsilyl)ethoxy]methyl}-1H-pyrrolo[2,3-b]pyridine (5 g, 68% purity) was recovered as a yellow oil.

¹H NMR (400 MHz, DMSO-d₆): δ=−0.11 (s, 9H), 0.80 (t, 2H), 1.19-1.16 (m, 2H), 1.37-1.36 (m, 2H), 3.54 (t, 2H), 5.54 (s, 2H), 5.83 (s, 2H), 6.32 (s, 1H), 6.40 (d, 1H), 7.79 (s, 1H).

### Intermediate 32

### 3,5-difluoro-4-[(3-[1-(trifluoromethyl)cyclopropyl]-1-{[2-(trimethylsilyl)ethoxy]methyl}-1H-pyrrolo[2,3-b]pyridin-4-yl)oxy]aniline

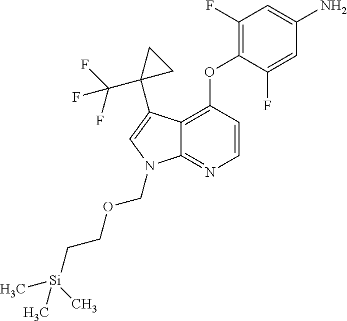 [SMILES: C[Si](C)(C)CCOCn1cc(C2(C(F)(F)F)CC2)c2c(Oc3c(F)cc(N)cc3F)ccnc21]

4-[(6-chloro-3-[1-(trifluoromethyl)cyclopropyl]-1-{[2-(trimethylsilyl)ethoxy]methyl}-1H-pyrrolo[2,3-b]pyridin-4-yl)oxy]-3,5-difluoroaniline (9 g, 86% purity and 3 g, crude, intermediate 31) in THF (200 mL) were added palladium on charcoal (2 g, 10% purity, containing 50% water) and triethylamine (10 mL, 71.9 mmol) under a nitrogen atmosphere. The suspension was degassed under vacuum and purged with hydrogen several times. The mixture was stirred under hydrogen (15 psi) at 45° C. for 36 hours. The mixture was filtered through a pad of Celite and the filtrate was concentrated in vacuum. The residue was dissolved in THF (200 mL) and palladium on charcoal (2 g, 10% purity, containing 50% water) was added. The mixture was stirred under hydrogen (15 psi) at 45° C. for 60 hours. The mixture was filtered through a pad of Celite, and the cake was washed with ethanol (100 mL×2). The filtrate was concentrated by evaporation in vacuum. The residue was purified by flash silica gel chromatography (0-10% of ethyl acetate in petroleum ether) to give the desired title compound (9 g, containing solvents residue) as brown oil. This product was combined with second batch of product (3 g, containing solvents residue) by dissolving in acetonitrile (200 mL). Water (100 mL) was added. The solution was concentrated by evaporation in vacuum to ˜150 mL. The residue was lyophilized to give the desired title compound (10.2 g) as a white solid.

¹H NMR (400 MHz, DMSO-d₆): δ=−0.11 (s, 9H), 0.82-0.78 (m, 2H), 1.17 (m, 2H), 1.38-1.35 (m, 2H), 3.54 (t, 2H), 5.59 (m, 2H), 5.76 (s, 2H), 6.42-6.34 (m, 3H), 7.73 (s, 1H), 8.11 (d, 1H).

¹⁹F NMR (400 MHz, DMSO-d₆): δ=−69, −129.

### Intermediate 33

### O-phenyl {3,5-difluoro-4-[(3-[1-(trifluoromethyl)cyclopropyl]-1-{[2-(trimethylsilyl)ethoxy]methyl}-1H-pyrrolo[2,3-b]pyridin-4-yl)oxy]phenyl}carbamothioate

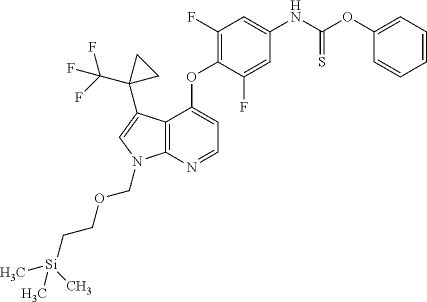 [SMILES: C[Si](C)(C)CCOCn1cc(C2(C(F)(F)F)CC2)c2c(Oc3c(F)cc(NC(=S)Oc4ccccc4)cc3F)ccnc21]

To a stirred solution of 3,5-difluoro-4-[(3-[1-(trifluoromethyl)cyclopropyl]-1-{[2-(trimethylsilyl)ethoxy]methyl}-1H-pyrrolo[2,3-b]pyridin-4-yl)oxy]aniline (500 mg, 1.00 mmol, intermediate 32) in a mixture of pyridine (750 μl, 9.3 mmol) and THF (7.5 mL) was added O-phenyl carbonochloridothioate (150 μl, 1.1 mmol, CAS No. [1005-56-7]). The reaction mixture was stirred at 0° C. for 1 h, at which time the solvent was evaporated to afford the crude material which was used in the next step without further purification.

LC-MS (Method 3): Rₜ=1.73 min; MS (ESIpos): m/z=636.5 [M+H]⁺

### Intermediate 34

### (+/−)-N-{3,5-difluoro-4-[(3-[1-(trifluoromethyl)cyclopropyl]-1-{[2-(trimethylsilyl)ethoxy]methyl}-1H-pyrrolo[2,3-b]pyridin-4-yl)oxy]phenyl}-N′-{[3-(hydroxymethyl)oxolan-3-yl]methyl}thiourea

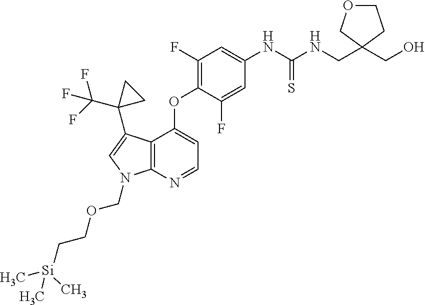 [SMILES: C[Si](C)(C)CCOCn1cc(C2(C(F)(F)F)CC2)c2c(Oc3c(F)cc(NC(=S)NCC4(CO)CCOC4)cc3F)ccnc21]

To a stirred solution of O-phenyl {3,5-difluoro-4-[(3-[1-(trifluoromethyl)cyclopropyl]-1-{[2-(trimethylsilyl)ethoxy]methyl}-1H-pyrrolo[2,3-b]pyridin-4-yl)oxy]phenyl}carbamothioate (300 mg, 472 μmol, intermediate 33) in DMF (12.0 mL) was added 3-(aminomethyl)oxolan-3-yl]methanol (124 mg, 944 μmol, CAS No. [1506738-56-2]). The resulting mixture was heated to 60° C. for 2 h at which time water and ethyl acetate were added and the layers were separated. The aqueous phase was extracted twice with ethyl acetate and the combined organic layers were washed with brine, dried over sodium sulfate, filtered and evaporated to give the crude product which was used without further purification.

LC-MS (Method 4): Rₜ=1.48 min; MS (ESIpos): m/z=674 [M+H]⁺

### Intermediate 35

### (+/−)-N-{3,5-difluoro-4-[(3-[1-(trifluoromethyl)cyclopropyl]-1-{[2-(trimethylsilyl)ethoxy]methyl}-1H-pyrrolo[2,3-b]pyridin-4-yl)oxy]phenyl}-2,7-dioxa-9-azaspiro[4.5]dec-8-en-8-amine

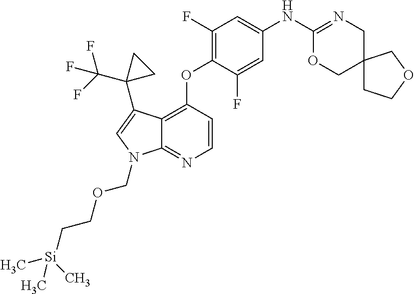 [SMILES: C[Si](C)(C)CCOCn1cc(C2(C(F)(F)F)CC2)c2c(Oc3c(F)cc(NC4=NCC5(CCOC5)CO4)cc3F)ccnc21]

To a solution of (+/−)-N-{3,5-difluoro-4-[(3-[1-(trifluoromethyl)cyclopropyl]-1-{[2-(trimethylsilyl)ethoxy]methyl}-1H-pyrrolo[2,3-b]pyridin-4-yl)oxy]phenyl}-N′-{[3-(hydroxymethyl)oxolan-3-yl]methyl}thiourea (310 mg, 461 μmol, Intermediate 34) in acetonitrile (5.0 mL) was added 1-(3-Dimethylaminopropyl)-3-ethylcarbodiimide hydrochloride (177 mg, 922 μmol) and triethylamine (190 μl, 1.4 mmol). The resulting mixture was stirred at 40° C. overnight at which time water and ethyl acetate were added and the layers separated. The aqueous phase was extracted twice with ethyl acetate and the combined organic layers were washed with brine, dried over sodium sulfate, filtered and evaporated to afford the crude product which was used without further purification.

LC-MS (Method 4): Rₜ=1.57 min; MS (ESIpos): m/z=639 [M+H]⁺

### Intermediate 36

### N-{3,5-difluoro-4-[(3-[1-(trifluoromethyl)cyclopropyl]-1-{[2-(trimethylsilyl)ethoxy]methyl}-1H-pyrrolo[2,3-b]pyridin-4-yl)oxy]phenyl}-N′-{[3-(hydroxymethyl)oxetan-3-yl]methyl}thiourea

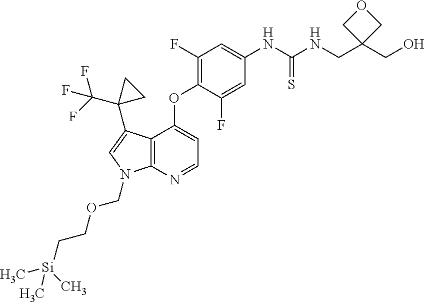 [SMILES: C[Si](C)(C)CCOCn1cc(C2(C(F)(F)F)CC2)c2c(Oc3c(F)cc(NC(=S)NCC4(CO)COC4)cc3F)ccnc21]

In analogy to Intermediate 34, O-phenyl {3,5-difluoro-4-[(3-[1-(trifluoromethyl)cyclopropyl]-1-{[2-(trimethylsilyl)ethoxy]methyl}-1H-pyrrolo[2,3-b]pyridin-4-yl)oxy]phenyl}carbamothioate (190 mg, 299 μmol, intermediate 33) and [3-(aminomethyl)oxetan-3-yl]methanol (70.0 mg, 598 μmol, CAS No. [45513-32-4]), in DMF (3.0 mL) were reacted to obtain a crude product which was used in the next step without further purification.

LC-MS (Method 4): Rₜ=1.47 min; MS (ESIpos): m/z=659 [M+H]⁺

### Intermediate 37

### methyl N′-{3,5-difluoro-4-[(3-[1-(trifluoromethyl)cyclopropyl]-1-{[2-(trimethylsilyl)ethoxy]methyl}-1H-pyrrolo[2,3-b]pyridin-4-yl)oxy]phenyl}-N-{[3-(hydroxymethyl)oxetan-3-yl]methyl}carbamimidothioate

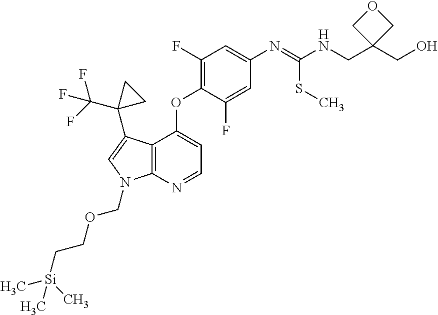 [SMILES: CS/C(=N\c1cc(F)c(Oc2ccnc3c2c(C2(C(F)(F)F)CC2)cn3COCC[Si](C)(C)C)c(F)c1)NCC1(CO)COC1]

To a stirred solution of N-{3,5-difluoro-4-[(3-[1-(trifluoromethyl)cyclopropyl]-1-{[2-(trimethylsilyl)ethoxy]methyl}-1H-pyrrolo[2,3-b]pyridin-4-yl)oxy]phenyl}-N′-{[3-(hydroxymethyl)oxetan-3-yl]methyl}thiourea (190 mg, 288 μmol, intermediate 36) in acetone (4.0 mL) was treated with N,N-diisopropylethylamine (250 μl, 1.4 mmol) followed by iodomethane (72 μl, 1.2 mmol). The reaction mixture was stirred at 55° C. for 2 h at which time the solvent was evaporated and the crude mixture used in the next step without further purification.

LC-MS (Method 4): Rₜ=1.58 min; MS (ESIpos): m/z=674 [M+H]⁺

### Intermediate 38

### N-{3,5-difluoro-4-[(3-[1-(trifluoromethyl)cyclopropyl]-1-{[2-(trimethylsilyl)ethoxy]methyl}-1H-pyrrolo[2,3-b]pyridin-4-yl)oxy]phenyl}-2,6-dioxa-8-azaspiro[3.5]non-7-en-7-amine

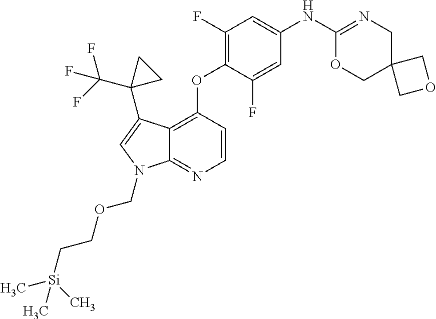 [SMILES: C[Si](C)(C)CCOCn1cc(C2(C(F)(F)F)CC2)c2c(Oc3c(F)cc(NC4=NCC5(COC5)CO4)cc3F)ccnc21]

To a stirred solution of methyl N′-{3,5-difluoro-4-[(3-[1-(trifluoromethyl)cyclopropyl]-1-{[2-(trimethylsilyl)ethoxy]methyl}-1H-pyrrolo[2,3-b]pyridin-4-yl)oxy]phenyl}-N-{[3-(hydroxymethyl)oxetan-3-yl]methyl}carbamimidothioate (190 mg, 282 μmol, intermediate 37) in tetrahydrofuran (3.0 mL) was added sodium hydroxide (28 mg, 706 μmol). The reaction mixture was stirred at room temperature for 2 hours, at which time water and ethyl acetate were added and the layers were separated. The aqueous phase was extracted 3 times with ethyl acetate and the combined organic layers were washed with brine, dried over sodium sulfate, filtered, and evaporated to afford the crude product which was used in the next step without further purification.

LC-MS (Method 4): Rₜ=1.55 min; MS (ESIpos): m/z=625 [M+H]⁺

### Intermediate 39

### N-{3,5-difluoro-4-[(3-[1-(trifluoromethyl)cyclopropyl]-1-{[2-(trimethylsilyl)ethoxy]methyl}-1H-pyrrolo[2,3-b]pyridin-4-yl)oxy]phenyl}-N′-(3-hydroxy-2,2-dimethylpropyl)thiourea

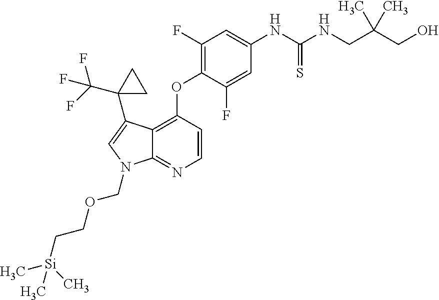 [SMILES: CC(C)(CO)CNC(=S)Nc1cc(F)c(Oc2ccnc3c2c(C2(C(F)(F)F)CC2)cn3COCC[Si](C)(C)C)c(F)c1]

In analogy to Intermediate 34, O-phenyl {3,5-difluoro-4-[(3-[1-(trifluoromethyl)cyclopropyl]-1-{[2-(trimethylsilyl)ethoxy]methyl}-1H-pyrrolo[2,3-b]pyridin-4-yl)oxy]phenyl}carbamothioate (300 mg, 472 μmol, intermediate 33) and 3-amino-2,2-dimethylpropan-1-ol (97.4 mg, 944 μmol, CAS No. [141-43-5]), in DMF (12 mL) were reacted to obtain a crude product which was used in the next step without further purification.

LC-MS (Method 4): Rₜ=1.56 min; MS (ESIpos): m/z=646 [M+H]⁺

### Intermediate 40

### N-{3,5-difluoro-4-[(3-[1-(trifluoromethyl)cyclopropyl]-1-{[2-(trimethylsilyl)ethoxy]methyl}-1H-pyrrolo[2,3-b]pyridin-4-yl)oxy]phenyl}-5,5-dimethyl-5,6-dihydro-4H-1,3-oxazin-2-amine

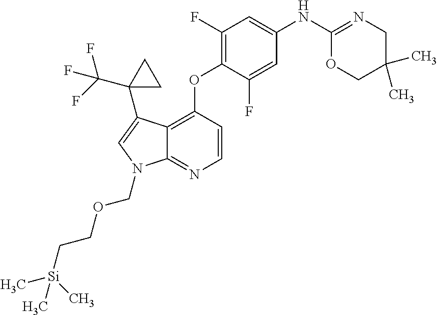

In analogy to intermediate 35, N-{3,5-difluoro-4-[(3-[1-(trifluoromethyl)cyclopropyl]-1-{[2-(trimethylsilyl)ethoxy]methyl}-1H-pyrrolo[2,3-b]pyridin-4-yl)oxy]phenyl}-N′-(3-hydroxy-2,2-dimethylpropyl)thiourea (300 mg, 465 μmol, intermediate 39) was reacted with 1-(3-Dimethylaminopropyl)-3-ethylcarbodiimide hydrochloride (178 mg, 931 μmol) and triethylamine (190 μl, 1.4 mmol) in acetonitrile (5.0 mL) to obtain a crude product which was used in the next step without further purification.

LC-MS (Method 4): Rₜ=1.67 min; MS (ESIpos): m/z=611 [M+H]⁺

### Intermediate 41

### N-{3,5-difluoro-4-[(3-[1-(trifluoromethyl)cyclopropyl]-1-{[2-(trimethylsilyl)ethoxy]methyl}-1H-pyrrolo[2,3-b]pyridin-4-yl)oxy]phenyl}-N′-{[1-(hydroxymethyl)cyclopropyl]methyl}thiourea

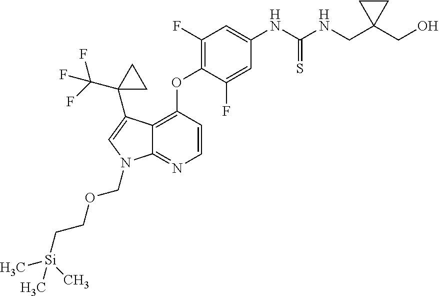 [SMILES: C[Si](C)(C)CCOCn1cc(C2(C(F)(F)F)CC2)c2c(Oc3c(F)cc(NC(=S)NCC4(CO)CC4)cc3F)ccnc21]

In analogy to Intermediate 34, O-phenyl {3,5-difluoro-4-[(3-[1-(trifluoromethyl)cyclopropyl]-1-{[2-(trimethylsilyl)ethoxy]methyl}-1H-pyrrolo[2,3-b]pyridin-4-yl)oxy]phenyl}carbamothioate (190 mg, 299 μmol, intermediate 33) and [1-(aminomethyl)cyclopropyl]methanol (60.5 mg, 598 μmol, CAS No. [45434-02-4]), in DMF (4.0 mL) were reacted to obtain a crude product which was used in the next step without further purification.

LC-MS (Method 4): Rₜ=1.55 min; MS (ESIpos): m/z=643 [M+H]⁺

### Intermediate 42

### N-{3,5-difluoro-4-[(3-[1-(trifluoromethyl)cyclopropyl]-1-{[2-(trimethylsilyl)ethoxy]methyl}-1H-pyrrolo[2,3-b]pyridin-4-yl)oxy]phenyl}-5-oxa-7-azaspiro[2.5]oct-6-en-6-amine

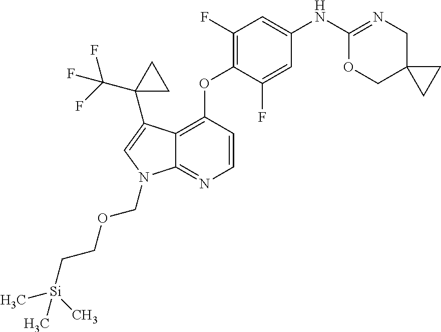 [SMILES: C[Si](C)(C)CCOCn1cc(C2(C(F)(F)F)CC2)c2c(Oc3c(F)cc(NC4=NCC5(CC5)CO4)cc3F)ccnc21]

In analogy to intermediate 35, N-{3,5-difluoro-4-[(3-[1-(trifluoromethyl)cyclopropyl]-1-{[2-(trimethylsilyl)ethoxy]methyl}-1H-pyrrolo[2,3-b]pyridin-4-yl)oxy]phenyl}-N′-{[1-(hydroxymethyl)cyclopropyl]methyl}thiourea (190 mg, 296 μmol, intermediate 41) was reacted with 1-(3-Dimethylaminopropyl)-3-ethylcarbodiimide hydrochloride (113 mg, 591 μmol) and triethylamine (120 μl, 890 μmol) in acetonitrile (4.0 mL) to obtain a crude product which was used in the next step without further purification.

LC-MS (Method 4): Rₜ=1.63 min; MS (ESIpos): m/z=610 [M+H]⁺

### Intermediate 43

### N-{3,5-difluoro-4-[(3-[1-(trifluoromethyl)cyclopropyl]-1-{[2-(trimethylsilyl)ethoxy]methyl}-1H-pyrrolo[2,3-b]pyridin-4-yl)oxy]phenyl}-N′-{[4-(hydroxymethyl)oxan-4-yl]methyl}thiourea

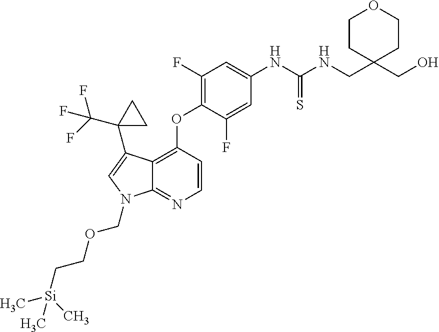 [SMILES: C[Si](C)(C)CCOCn1cc(C2(C(F)(F)F)CC2)c2c(Oc3c(F)cc(NC(=S)NCC4(CO)CCOCC4)cc3F)ccnc21]

In analogy to Intermediate 34, O-phenyl {3,5-difluoro-4-[(3-[1-(trifluoromethyl)cyclopropyl]-1-{[2-(trimethylsilyl)ethoxy]methyl}-1H-pyrrolo[2,3-b]pyridin-4-yl)oxy]phenyl}carbamothioate (190 mg, 299 μmol, intermediate 33) and [4-(aminomethyl)oxan-4-yl]methanol (87 mg, 0.60 mmol, CAS No. [959238-22-3]), in DMF (4.0 mL) were reacted to obtain a crude product which was used in the next step without further purification.

LC-MS (Method 4): Rₜ=1.50 min; MS (ESIpos): m/z=688 [M+H]⁺

### Intermediate 44

### N-{3,5-difluoro-4-[(3-[1-(trifluoromethyl)cyclopropyl]-1-{[2-(trimethylsilyl)ethoxy]methyl}-1H-pyrrolo[2,3-b]pyridin-4-yl)oxy]phenyl}-2,9-dioxa-4-azaspiro[5.5]undec-3-en-3-amine

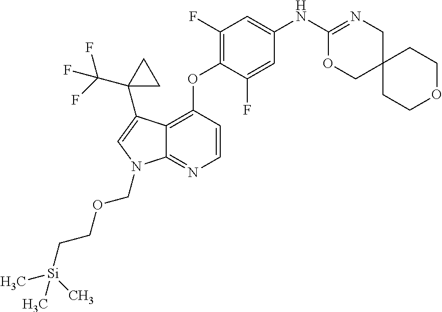 [SMILES: C[Si](C)(C)CCOCn1cc(C2(C(F)(F)F)CC2)c2c(Oc3c(F)cc(NC4=NCC5(CCOCC5)CO4)cc3F)ccnc21]

In analogy to intermediate 35, N-{3,5-difluoro-4-[(3-[1-(trifluoromethyl)cyclopropyl]-1-{[2-(trimethylsilyl)ethoxy]methyl}-1H-pyrrolo[2,3-b]pyridin-4-yl)oxy]phenyl}-N′-{[4-(hydroxymethyl)oxan-4-yl]methyl}thiourea (200 mg, 0.29 mmol, intermediate 43) was reacted with 1-(3-Dimethylaminopropyl)-3-ethylcarbodiimide hydrochloride (112 mg, 582 μmol) and triethylamine (122 μl, 726 μmol) in acetonitrile (4.0 mL) to obtain a crude product which was used in the next step without further purification.

LC-MS (Method 4): Rₜ=1.59 min; MS (ESIpos): m/z=654 [M+H]⁺

### Intermediate 45

### N-{3,5-difluoro-4-[(3-[1-(trifluoromethyl)cyclopropyl]-1-{[2-(trimethylsilyl)ethoxy]methyl}-1H-pyrrolo[2,3-b]pyridin-4-yl)oxy]phenyl}-N′-{[1-(hydroxymethyl)cyclobutyl]methyl}thiourea

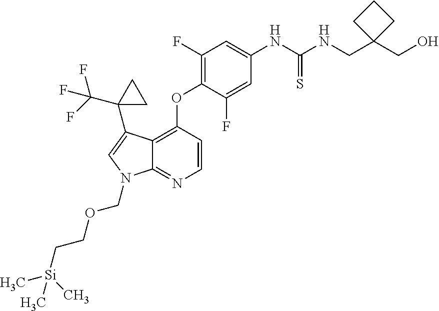 [SMILES: C[Si](C)(C)CCOCn1cc(C2(C(F)(F)F)CC2)c2c(Oc3c(F)cc(NC(=S)NCC4(CO)CCC4)cc3F)ccnc21]

In analogy to Intermediate 34, O-phenyl {3,5-difluoro-4-[(3-[1-(trifluoromethyl)cyclopropyl]-1-{[2-(trimethylsilyl)ethoxy]methyl}-1H-pyrrolo[2,3-b]pyridin-4-yl)oxy]phenyl}carbamothioate (300 mg, 472 μmol, intermediate 33) and [1-(aminomethyl)cyclobutyl]methanol (109 mg, 944 μmol, CAS No. [2041-56-7]), in DMF (12 mL) were reacted to obtain a crude product which was used in the next step without further purification.

LC-MS (Method 4): Rₜ=1.57 min; MS (ESIpos): m/z=658 [M+H]⁺

### Intermediate 46

### N-{3,5-difluoro-4-[(3-[1-(trifluoromethyl)cyclopropyl]-1-{[2-(trimethylsilyl)ethoxy]methyl}-1H-pyrrolo[2,3-b]pyridin-4-yl)oxy]phenyl}-6-oxa-8-azaspiro[3.5]non-7-en-7-amine

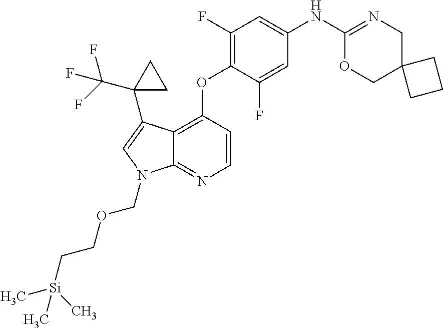 [SMILES: C[Si](C)(C)CCOCn1cc(C2(C(F)(F)F)CC2)c2c(Oc3c(F)cc(NC4=NCC5(CCC5)CO4)cc3F)ccnc21]

In analogy to intermediate 35, N-{3,5-difluoro-4-[(3-[1-(trifluoromethyl)cyclopropyl]-1-{[2-(trimethylsilyl)ethoxy]methyl}-1H-pyrrolo[2,3-b]pyridin-4-yl)oxy]phenyl}-N′-{[1-(hydroxymethyl)cyclobutyl]methyl}thiourea (300 mg, 457 μmol), intermediate 45) was reacted with 1-(3-Dimethylaminopropyl)-3-ethylcarbodiimide hydrochloride (175 mg, 914 μmol) and triethylamine (190 μl, 1.4 mmol) in acetonitrile (5.0 mL) to obtain a crude product which was used in the next step without further purification.

LC-MS (Method 4): Rₜ=1.68 min; MS (ESIpos): m/z=623 [M+H]⁺

### Intermediate 47

### (+/−)-N-{3,5-difluoro-4-[(3-[1-(trifluoromethyl)cyclobutyl]-1-{[2-(trimethylsilyl)ethoxy]methyl}-1H-pyrrolo[2,3-b]pyridin-4-yl)oxy]phenyl}-N′-{[3-(hydroxymethyl)oxolan-3-yl]methyl}thiourea

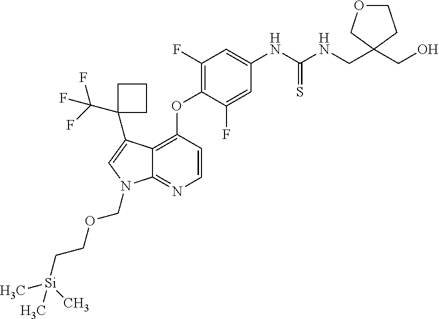 [SMILES: C[Si](C)(C)CCOCn1cc(C2(C(F)(F)F)CCC2)c2c(Oc3c(F)cc(NC(=S)NCC4(CO)CCOC4)cc3F)ccnc21]

In analogy to Intermediate 34, O-phenyl {3,5-difluoro-4-[(3-[1-(trifluoromethyl)cyclobutyl]-1-{[2-(trimethylsilyl)ethoxy]methyl}-1H-pyrrolo[2,3-b]pyridin-4-yl)oxy]phenyl}carbamothioate (100 mg, 154 μmol, intermediate 14) and 3-(aminomethyl)oxolan-3-yl]methanol (40.4 mg, 308 μmol, CAS No. [1506738-56-2]), in DMF (2.0 mL) were reacted to obtain a crude product which was used in the next step without further purification.

LC-MS (Method 4): Rₜ=1.55 min; MS (ESIpos): m/z=688 [M+H]⁺

### Intermediate 48

### (+/−)-N-{3,5-difluoro-4-[(3-[1-(trifluoromethyl)cyclobutyl]-1-{[2-(trimethylsilyl)ethoxy]methyl}-1H-pyrrolo[2,3-b]pyridin-4-yl)oxy]phenyl}-2,7-dioxa-9-azaspiro[4.5]dec-8-en-8-amine

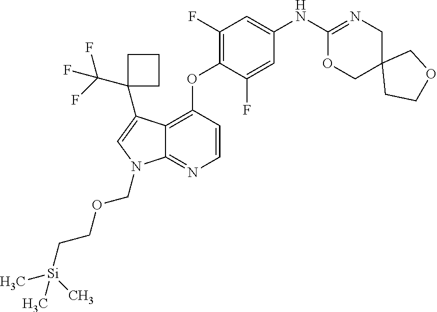 [SMILES: C[Si](C)(C)CCOCn1cc(C2(C(F)(F)F)CCC2)c2c(Oc3c(F)cc(NC4=NCC5(CCOC5)CO4)cc3F)ccnc21]

In analogy to intermediate 35, (+/−)-N-{3,5-difluoro-4-[(3-[1-(trifluoromethyl)cyclobutyl]-1-{[2-(trimethylsilyl)ethoxy]methyl}-1H-pyrrolo[2,3-b]pyridin-4-yl)oxy]phenyl}-N′-{[3-(hydroxymethyl)oxolan-3-yl]methyl}thiourea (100 mg, 146 μmol, intermediate 47) was reacted with 1-(3-Dimethylaminopropyl)-3-ethylcarbodiimide hydrochloride (55.8 mg, 291 μmol) and triethylamine (61 μl, 440 μmol) in acetonitrile (2.0 mL) to obtain a crude product which was used in the next step without further purification.

LC-MS (Method 4): Rₜ=1.62 min; MS (ESIpos): m/z=654 [M+H]⁺

### Intermediate 49

### N-{3,5-difluoro-4-[(3-[1-(trifluoromethyl)cyclobutyl]-1-{[2-(trimethylsilyl)ethoxy]methyl}-1H-pyrrolo[2,3-b]pyridin-4-yl)oxy]phenyl}-N′-{[3-(hydroxymethyl)oxetan-3-yl]methyl}thiourea

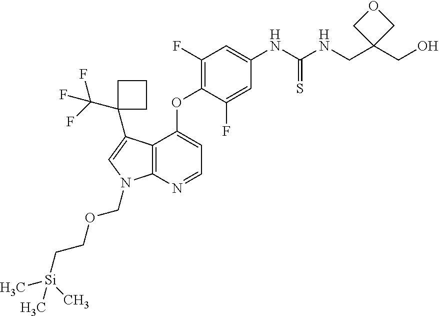 [SMILES: C[Si](C)(C)CCOCn1cc(C2(C(F)(F)F)CCC2)c2c(Oc3c(F)cc(NC(=S)NCC4(CO)COC4)cc3F)ccnc21]

In analogy to Intermediate 34, O-phenyl {3,5-difluoro-4-[(3-[1-(trifluoromethyl)cyclobutyl]-1-{[2-(trimethylsilyl)ethoxy]methyl}-1H-pyrrolo[2,3-b]pyridin-4-yl)oxy]phenyl}carbamothioate (190 mg, 292 μmol, intermediate 14) and [3-(aminomethyl)oxetan-3-yl]methanol (68.5 mg, 585 μmol, CAS No. [45513-32-4]), in DMF (3.0 mL) were reacted to obtain a crude product which was used in the next step without further purification.

LC-MS (Method 4): Rₜ=1.51 min; MS (ESIpos): m/z=674 [M+H]⁺

### Intermediate 50

### methyl N′-{3,5-difluoro-4-[(3-[1-(trifluoromethyl)cyclobutyl]-1-{[2-(trimethylsilyl)ethoxy]methyl}-1H-pyrrolo[2,3-b]pyridin-4-yl)oxy]phenyl}-N-{[3-(hydroxymethyl)oxetan-3-yl]methyl}carbamimidothioate

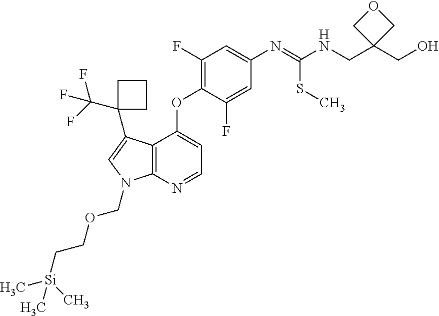 [SMILES: CS/C(=N\c1cc(F)c(Oc2ccnc3c2c(C2(C(F)(F)F)CCC2)cn3COCC[Si](C)(C)C)c(F)c1)NCC1(CO)COC1]

To a stirred solution of N-{3,5-difluoro-4-[(3-[1-(trifluoromethyl)cyclobutyl]-1-{[2-(trimethylsilyl)ethoxy]methyl}-1H-pyrrolo[2,3-b]pyridin-4-yl)oxy]phenyl}-N′-{[3-(hydroxymethyl)oxetan-3-yl]methyl}thiourea (190 mg, 282 μmol, intermediate 49) in acetone (4.0 mL) was treated with N,N-diisopropylethylamine (250 μl, 1.4 mmol) followed by iodomethane (70 μl, 1.1 mmol). The reaction mixture was stirred at 55° C. for 2 h at which time the solvent was evaporated and the crude mixture used in the next step without further purification.

LC-MS (Method 4): Rₜ=1.62 min; MS (ESIpos): m/z=688 [M+H]⁺

### Intermediate 51

### N-{3,5-difluoro-4-[(3-[1-(trifluoromethyl)cyclobutyl]-1-{[2-(trimethylsilyl)ethoxy]methyl}-1H-pyrrolo[2,3-b]pyridin-4-yl)oxy]phenyl}-2,6-dioxa-8-azaspiro[3.5]non-7-en-7-amine

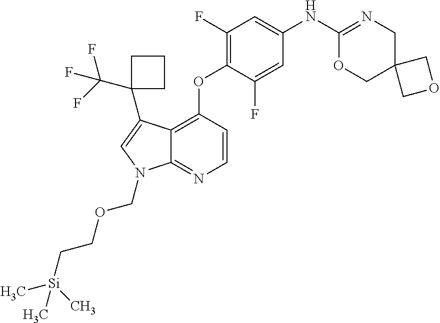 [SMILES: C[Si](C)(C)CCOCn1cc(C2(C(F)(F)F)CCC2)c2c(Oc3c(F)cc(NC4=NCC5(COC5)CO4)cc3F)ccnc21]

To a stirred solution of methyl N′-{3,5-difluoro-4-[(3-[1-(trifluoromethyl)cyclobutyl]-1-{[2-(trimethylsilyl)ethoxy]methyl}-1H-pyrrolo[2,3-b]pyridin-4-yl)oxy]phenyl}-N-{[3-(hydroxymethyl)oxetan-3-yl]methyl}carbamimidothioate (190 mg, 277 μmol, intermediate 50) in tetrahydrofuran (3.0 mL) was added sodium hydroxide (28 mg, 706 μmol). The reaction mixture was stirred at room temperature for 2 hours, at which time water and ethyl acetate were added and the layers were separated. The aqueous phase was extracted 3 times with ethyl acetate and the combined organic layers were washed with brine, dried over sodium sulfate, filtered, and evaporated to afford the crude product which was used in the next step without further purification.

LC-MS (Method 4): Rₜ=1.58 min; MS (ESIpos): m/z=639 [M+H]⁺

### EXAMPLE 1

### N-[3,5-difluoro-4-({3-[1-(trifluoromethyl)cyclobutyl]-1H-pyrrolo[2,3-b]pyridin-4-yl}oxy)phenyl]-6-oxa-8-azaspiro[3.5]non-7-en-7-amine

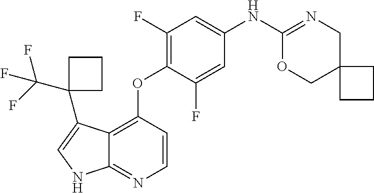 [SMILES: Fc1cc(NC2=NCC3(CCC3)CO2)cc(F)c1Oc1ccnc2[nH]cc(C3(C(F)(F)F)CCC3)c12]

N-[3,5-difluoro-4-({3-[1-(trifluoromethyl)cyclobutyl]-1H-pyrrolo[2,3-b]pyridin-4-yl}oxy)phenyl]-N′-{[1-(hydroxymethyl)cyclobutyl]methyl}thiourea (Intermediate 16, 90.0 mg, 166 μmol), 1-(3-dimethylaminopropyl)-3-ethylcarbodiimide hydrochloride (63.8 mg, 333 μmol), and trimethylamine (70 μL, 500 μmol) were dissolved in acetonitrile (1 mL) and stirred overnight at 40° C. Water was added, and the mixture extracted with ethyl acetate. The organic layers were dried over sodium sulfate, filtered and the solvent removed under vacuum. The crude product was purified by silica gel chromatography and digested in hexanes/diethyl ether to yield the title compound (52 mg, 59% yield).

LC-MS (Method 4): Rₜ=1.36 min; MS (ESIpos): m/z=507 [M+H]⁺

¹H-NMR (400 MHz, DMSO-d6) δ [ppm]: 11.93 (d, 1H), 8.98 (br s, 1H), 8.04 (d, 1H), 7.50 (d, 1H), 7.48 (br s, 2H), 6.23 (d, 1H), 4.13 (s, 2H), 3.30 (m, 2H), 2.66 (m, 4H), 2.08-1.78 (m, 8H).

### EXAMPLE 2

### N-[3,5-difluoro-4-({3-[1-(trifluoromethyl)cyclobutyl]-1H-pyrrolo[2,3-b]pyridin-4-yl}oxy)phenyl]-2,9-dioxa-4-azaspiro[5.5]undec-3-en-3-amine

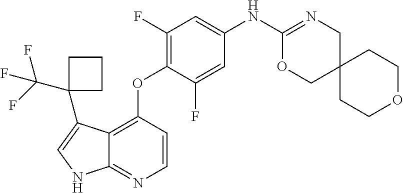 [SMILES: Fc1cc(NC2=NCC3(CCOCC3)CO2)cc(F)c1Oc1ccnc2[nH]cc(C3(C(F)(F)F)CCC3)c12]

N-[3,5-difluoro-4-({3-[1-(trifluoromethyl)cyclobutyl]-1H-pyrrolo[2,3-b]pyridin-4-yl}oxy)phenyl]-N′-{[4-(hydroxymethyl)oxan-4-yl]methyl}thiourea (Intermediate 18, 90.0 mg, 158 μmol), 1-(3-dimethylaminopropyl)-3-ethylcarbodiimide hydrochloride (60.5 mg, 315 μmol), and triethylamine (66 μl, 470 μmol) were dissolved in acetonitrile (950 μL) and stirred overnight at 40° C. Water was added, and the mixture extracted with ethyl acetate. The organic layers were dried over sodium sulfate, filtered and the solvent removed under vacuum. The crude product was purified by silica gel chromatography and digested in hexanes/diethyl ether to yield the title compound (39 mg, 44% yield).

LC-MS (Method 4): Rₜ=1.22 min; MS (ESIpos): m/z=537 [M+H]⁺

¹H-NMR (400 MHz, DMSO-d6) δ [ppm]: 11.93 (d, 1H), 9.02 (br s, 1H), 8.04 (d, 1H), 7.53 (br s, 2H), 7.50 (d, 1H), 6.25 (d, 1H), 4.06 (s, 2H), 3.60 (m, 4H), 3.26 (br s, 2H), 2.66 (br t, 4H), 2.05-1.90 (m, 2H), 1.43 (br s, 4H).

### EXAMPLE 3

### N-[3,5-difluoro-4-({3-[1-(trifluoromethyl)cyclobutyl]-1H-pyrrolo[2,3-b]pyridin-4-yl}oxy)phenyl]-5-oxa-7-azaspiro[2.5]oct-6-en-6-amine

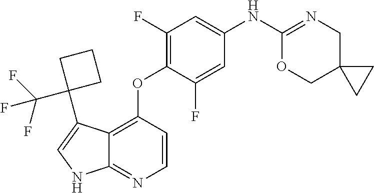 [SMILES: Fc1cc(NC2=NCC3(CC3)CO2)cc(F)c1Oc1ccnc2[nH]cc(C3(C(F)(F)F)CCC3)c12]

N-{3,5-difluoro-4-[(3-[1-(trifluoromethyl)cyclobutyl]-1-{[2-(trimethylsilyl)ethoxy]methyl}-1H-pyrrolo[2,3-b]pyridin-4-yl)oxy]phenyl}-5-oxa-7-azaspiro[2.5]oct-6-en-6-amine (Intermediate 20, 119 mg, 191 μmol) and trifluoroacetic acid (370 μl, 4.8 mmol) were dissolved in dichloromethane (2 mL) and stirred at room temperature for 5 h. Acetonitrile (2 mL) and aqueous ammonia (33%, 1 mL) were added, and the mixture stirred for 1 h. The solvent was removed under vacuum and the residue purified by preparative HPLC to yield the title compound (16 mg, 16% yield).

LC-MS (Method 4): Rₜ=1.29 min; MS (ESIpos): m/z=493 [M+H]⁺

¹H-NMR (400 MHz, DMSO-d6) δ [ppm]: 11.93 (d, 1H), 8.98 (br s, 1H), 8.05 (d, 1H), 7.55 (br s, 2H), 7.49 (d, 1H), 6.24 (d, 1H), 4.02 (s, 2H), 3.20 (br s, 2H), 2.66 (br t, 4H), 2.06-1.88 (m, 2H), 0.61 (br s, 2H), 0.56 (br s, 2H).

### EXAMPLE 4

### N-[3,5-difluoro-4-({3-[1-(trifluoromethyl)cyclobutyl]-1H-pyrrolo[2,3-b]pyridin-4-yl}oxy)phenyl]-5,5-dimethyl-5,6-dihydro-4H-1,3-oxazin-2-amine

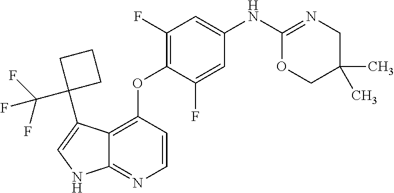 [SMILES: CC1(C)CN=C(Nc2cc(F)c(Oc3ccnc4[nH]cc(C5(C(F)(F)F)CCC5)c34)c(F)c2)OC1]

N-{3,5-difluoro-4-[(3-[1-(trifluoromethyl)cyclobutyl]-1-{[2-(trimethylsilyl)ethoxy]methyl}-1H-pyrrolo[2,3-b]pyridin-4-yl)oxy]phenyl}-5,5-dimethyl-5,6-dihydro-4H-1,3-oxazin-2-amine (Intermediate 22, 124 mg, 198 μmol) and trifluoroacetic acid (380 μl, 5.0 mmol) were dissolved in dichloromethane (2 mL) and stirred at room temperature for 5 h. Acetonitrile (2 mL) and aqueous ammonia (33%, 1 mL) were added, and the mixture stirred for 1 h. The solvent was removed under vacuum and the residue purified by preparative HPLC to yield the title compound (20 mg, 20% yield).

LC-MS (Method 4): Rₜ=1.33 min; MS (ESIpos): m/z=495 [M+H]⁺

¹H-NMR (400 MHz, DMSO-d6) δ [ppm]: 11.93 (d, 1H), 9.00 (br s, 1H), 8.05 (d, 1H), 7.55 (br s, 2H), 7.50 (d, 1H), 6.25 (d, 1H), 3.87 (br s, 2H), 3.07 (br s, 2H), 2.66 (br t, 4H), 2.05-1.90 (m, 2H), 0.96 (s, 6H).

### EXAMPLE 5

### (+/−)-N-[3,5-difluoro-4-({3-[1-(trifluoromethyl)cyclopropyl]-1H-pyrrolo[2,3-b]pyridin-4-yl}oxy)phenyl]-2,7-dioxa-9-azaspiro[4.5]dec-8-en-8-amine

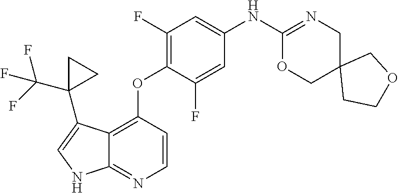 [SMILES: Fc1cc(NC2=NCC3(CCOC3)CO2)cc(F)c1Oc1ccnc2[nH]cc(C3(C(F)(F)F)CC3)c12]

To a solution of (+/−)-N-{3,5-difluoro-4-[(3-[1-(trifluoromethyl)cyclopropyl]-1-{[2-(trimethylsilyl)ethoxy]methyl}-1H-pyrrolo[2,3-b]pyridin-4-yl)oxy]phenyl}-2,7-dioxa-9-azaspiro[4.5]dec-8-en-8-amine (600 mg, 0.94 mmol, intermediate 35) in dichloromethane (4.0 mL) was added trifluoroacetic acid (4.0 mL). The reaction mixture was stirred at room temperature overnight, at which time the mixture was basificed to pH >10 with 2M sodium hydroxide and ethyl acetate was added. The layers were separated and the aqueous layer was extracted three times with ethyl acetate. The combined organic layers were washed with brine, dried over sodium sulfate, filtered, and evaporated. The crude material was dissolved in acetonitrile (10 mL) and treated with a 25% aqueous solution of ammonia (5 mL). The resulting solution was stirred for 1 hour and then purified by preparative HPLC to afford the title compound (284 mg, 59% yield).

LC-MS (Method 4): Rₜ=1.15 min; MS (ESIpos): m/z=509 [M+H]⁺

¹H NMR (400 MHz, DMSO-d₆) 5 ppm 1.18 (br s, 2H), 1.30-1.37 (m, 2H), 1.65-1.82 (m, 2H), 3.41-3.50 (m, 1H), 3.60 (d, 1H), 3.74-3.85 (m, 2H), 4.10 (q, 2H), 6.26 (d, 1H), 7.53 (d, 1H), 7.57 (br s, 1H), 8.04 (d, 1H), 9.07 (br s, 1H), 11.91 (br d, 1H)

The title compound was separated into its enantiomers by preparative chiral SFC to give stereoisomer 1 (76 mg, see example 6), stereoisomer 2 (58 mg, see example 7). For the isolation of stereoisomer 1, and stereoisomer 2 the following method was used.

Analytical chiral SFC method:

Instrument: Agilent: 1260, Aurora SFC-Module; Column: Chiralpak IC 5μ 100×4.6 mm; Eluent A: CO₂; Eluent B: Ethanol+0.1 Vol-% NH₄OH (32%); Isocratic: 20% B; Flowrate: 4 ml/min; Temperature: 37.5° C.; Pressure: 100 bar; UV: 254 nm Preparative chiral SFC method:

Instrument: Sepiatec: Prep SFC100; Column: Chiralpak IC 5μ 250×30 mm; Eluent A: CO₂; Eluent B: Ethanol+0.2 Vol-% NH₄OH (32%); Isocractic: 20% B; Flowrate: 100 ml/min; Temperature: 40° C.; Pressure: 150 bar; UV: 254 nm

### EXAMPLE 6, Example 7

### (5R)—N-[3,5-difluoro-4-({3-[1-(trifluoromethyl)cyclopropyl]-1H-pyrrolo[2,3-b]pyridin-4-yl}oxy)phenyl]-2,7-dioxa-9-azaspiro[4.5]dec-8-en-8-amine

### (5S)—N-[3,5-difluoro-4-({3-[1-(trifluoromethyl)cyclopropyl]-1H-pyrrolo[2,3-b]pyridin-4-yl}oxy)phenyl]-2,7-dioxa-9-azaspiro[4.5]dec-8-en-8-amine

### EXAMPLE 6

### N-[3,5-difluoro-4-({3-[1-(trifluoromethyl)cyclopropyl]-1H-pyrrolo[2,3-b]pyridin-4-yl}oxy)phenyl]-2,7-dioxa-9-azaspiro[4.5]dec-8-en-8-amine (single enantiomer 1)

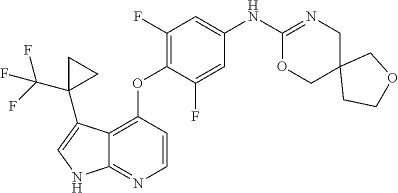 [SMILES: Fc1cc(NC2=NCC3(CCOC3)CO2)cc(F)c1Oc1ccnc2[nH]cc(C3(C(F)(F)F)CC3)c12]

For the preparation of the title compound and separation into its isomers, see example 5. Analytical chiral HPLC (method, see example 5): Rt=2.10 min, ee=97.7%

### EXAMPLE 7

### N-[3,5-difluoro-4-({3-[1-(trifluoromethyl)cyclopropyl]-1H-pyrrolo[2,3-b]pyridin-4-yl}oxy)phenyl]-2,7-dioxa-9-azaspiro[4.5]dec-8-en-8-amine (single enantiomer 2)

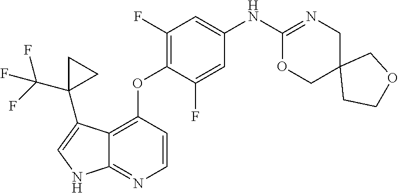 [SMILES: Fc1cc(NC2=NCC3(CCOC3)CO2)cc(F)c1Oc1ccnc2[nH]cc(C3(C(F)(F)F)CC3)c12]

For the preparation of the title compound and separation into its isomers, see example 5. Analytical chiral HPLC (method, see example 5): Rt=2.62 min, ee=96.2%

### EXAMPLE 8

### N-[3,5-difluoro-4-({3-[1-(trifluoromethyl)cyclopropyl]-1H-pyrrolo[2,3-b]pyridin-4-yl}oxy)phenyl]-2,6-dioxa-8-azaspiro[3.5]non-7-en-7-amine

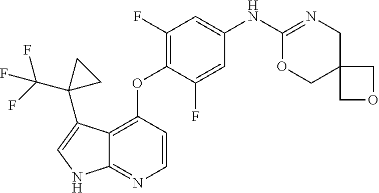 [SMILES: Fc1cc(NC2=NCC3(COC3)CO2)cc(F)c1Oc1ccnc2[nH]cc(C3(C(F)(F)F)CC3)c12]

In analogy to Example 5, N-{3,5-difluoro-4-[(3-[1-(trifluoromethyl)cyclopropyl]-1-{[2-(trimethylsilyl)ethoxy]methyl}-1H-pyrrolo[2,3-b]pyridin-4-yl)oxy]phenyl}-2,6-dioxa-8-azaspiro[3.5]non-7-en-7-amine (175 mg, 280 μmol, intermediate 38) was treated with trifluoroacetic acid (1.0 mL) in dichloromethane (2.0 mL) to afford after preparative HPLC purification the title compound (46 mg, 32% yield).

LC-MS (Method 4): Rₜ=1.10 min; MS (ESIpos): m/z=495 [M+H]⁺

¹H NMR (400 MHz, DMSO-d₆) 5 ppm 1.18 (br s, 2H), 1.31-1.36 (m, 2H), 3.60 (s, 2H), 4.39-4.45 (m, 6H), 6.24 (d, 1H), 7.41 (br s, 2H), 7.53 (s, 1H), 8.03 (d, 1H), 11.91 (br s, 1H)

### EXAMPLE 9

### N-[3,5-difluoro-4-({3-[1-(trifluoromethyl)cyclopropyl]-1H-pyrrolo[2,3-b]pyridin-4-yl}oxy)phenyl]-5,5-dimethyl-5,6-dihydro-4H-1,3-oxazin-2-amine

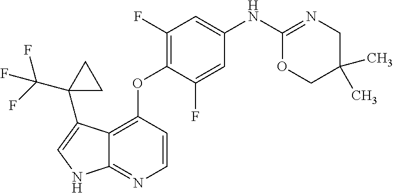 [SMILES: CC1(C)CN=C(Nc2cc(F)c(Oc3ccnc4[nH]cc(C5(C(F)(F)F)CC5)c34)c(F)c2)OC1]

In analogy to Example 5, N-{3,5-difluoro-4-[(3-[1-(trifluoromethyl)cyclopropyl]-1-{[2-(trimethylsilyl)ethoxy]methyl}-1H-pyrrolo[2,3-b]pyridin-4-yl)oxy]phenyl}-5,5-dimethyl-5,6-dihydro-4H-1,3-oxazin-2-amine (280 mg, 458 μmol, intermediate 40) was treated with trifluoroacetic acid (2.0 mL) in dichloromethane (2.0 mL) to afford after preparative HPLC purification the title compound (82 mg, 37% yield).

LC-MS (Method 4): Rₜ=1.28 min; MS (ESIpos): m/z=482 [M+H]⁺

¹H NMR (400 MHz, DMSO-d₆) 5 ppm 0.96 (br s, 6H), 1.18 (br s, 2H), 1.30-1.36 (m, 2H), 3.10 (br s, 2H), 3.87 (br s, 2H), 6.27 (d, 1H), 7.53 (d, 1H), 7.58 (br s, 1H), 8.04 (d, 1H), 9.01 (br s, 1H), 11.91 (brd, 1H)

### EXAMPLE 10

### N-[3,5-difluoro-4-({3-[1-(trifluoromethyl)cyclopropyl]-1H-pyrrolo[2,3-b]pyridin-4-yl}oxy)phenyl]-5-oxa-7-azaspiro[2.5]oct-6-en-6-amine

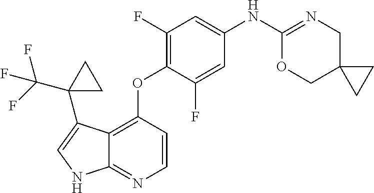 [SMILES: Fc1cc(NC2=NCC3(CC3)CO2)cc(F)c1Oc1ccnc2[nH]cc(C3(C(F)(F)F)CC3)c12]

In analogy to Example 5, N-{3,5-difluoro-4-[(3-[1-(trifluoromethyl)cyclopropyl]-1-{[2-(trimethylsilyl)ethoxy]methyl}-1H-pyrrolo[2,3-b]pyridin-4-yl)oxy]phenyl}-5-oxa-7-azaspiro[2.5]oct-6-en-6-amine (175 mg, 288 μmol, intermediate 42) was treated with trifluoroacetic acid (1.0 mL) in dichloromethane (2.0 mL) to afford after preparative HPLC purification the title compound (28 mg, 19% yield).

LC-MS (Method 4): Rₜ=1.25 min; MS (ESIpos): m/z=479.7 [M+H]⁺

¹H NMR (400 MHz, DMSO-d₆) δ ppm 0.59 (br d, 4H), 1.18 (br s, 2H), 1.31-1.37 (m, 2H), 3.21 (br s, 2H), 4.02 (s, 2H), 6.25 (d, 1H), 7.53 (s, 1H), 7.57 (br s, 1H), 8.04 (d, 1H), 8.99 (br s, 1H), 11.91 (br s, 1H)

### EXAMPLE 11

### N-[3,5-difluoro-4-({3-[1-(trifluoromethyl)cyclopropyl]-1H-pyrrolo[2,3-b]pyridin-4-yl}oxy)phenyl]-2,9-dioxa-4-azaspiro[5.5]undec-3-en-3-amine

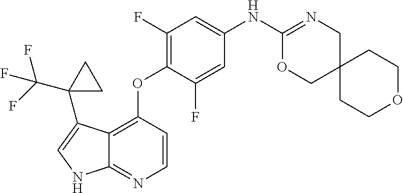 [SMILES: Fc1cc(NC2=NCC3(CCOCC3)CO2)cc(F)c1Oc1ccnc2[nH]cc(C3(C(F)(F)F)CC3)c12]

To a solution of N-{3,5-difluoro-4-[(3-[1-(trifluoromethyl)cyclopropyl]-1-{[2-(trimethylsilyl)ethoxy]methyl}-1H-pyrrolo[2,3-b]pyridin-4-yl)oxy]phenyl}-2,9-dioxa-4-azaspiro[5.5]undec-3-en-3-amine (190 mg, 291 μmol, intermediate 44) in dichloromethane (2.0 mL) was added trifluoroacetic acid (1.0 mL). The reaction mixture was stirred at room temperature overnight, at which time the mixture was basificed to pH >10 with 2M sodium hydroxide and ethyl acetate was added. The layers were separated and the aqueous layer was extracted three times with ethyl acetate. The combined organic layers were washed with brine, dried over sodium sulfate, filtered, and evaporated. The crude material was dissolved in acetonitrile (10 mL) and treated with a 25% aqueous solution of ammonia (5 mL). The resulting solution was stirred for 1 hour and then purified by preparative HPLC to afford the title compound (38 mg, 24% yield).

LC-MS (Method 4): Rₜ=1.16 min; MS (ESIpos): m/z=524 [M+H]⁺

¹H NMR (400 MHz, DMSO-d₆) δ ppm 1.18 (br s, 2H), 1.31-1.35 (m, 2H), 1.44 (br s, 4H), 3.28 (br d, 2H), 3.54-3.67 (m, 4H), 4.07 (br s, 2H), 6.26 (d, 1H), 7.53 (s, 1H), 7.57 (br s, 1H), 8.04 (d, 1H), 9.03 (br s, 1H), 11.91 (s, 1H)

### EXAMPLE 12

### N-[3,5-difluoro-4-({3-[1-(trifluoromethyl)cyclopropyl]-1H-pyrrolo[2,3-b]pyridin-4-yl}oxy)phenyl]-6-oxa-8-azaspiro[3.5]non-7-en-7-amine

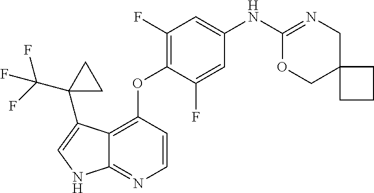 [SMILES: Fc1cc(NC2=NCC3(CCC3)CO2)cc(F)c1Oc1ccnc2[nH]cc(C3(C(F)(F)F)CC3)c12]

In analogy to Example 5, N-{3,5-difluoro-4-[(3-[1-(trifluoromethyl)cyclopropyl]-1-{[2-(trimethylsilyl)ethoxy]methyl}-1H-pyrrolo[2,3-b]pyridin-4-yl)oxy]phenyl}-6-oxa-8-azaspiro[3.5]non-7-en-7-amine (280 mg, 450 μmol, intermediate 46) was treated with trifluoroacetic acid (2.0 mL) in dichloromethane (2.0 mL) to afford after preparative HPLC purification the title compound (68 mg, 30% yield).

LC-MS (Method 4): Rₜ=1.31 min; MS (ESIpos): m/z=494 [M+H]⁺

¹H NMR (400 MHz, DMSO-d₆) δ ppm 1.18 (br s, 2H), 1.31-1.36 (m, 2H), 1.78-2.02 (m, 6H), 3.31 (br s, 2H), 4.14 (s, 2H), 6.26 (d, 1H), 7.53 (d, 1H), 8.04 (d, 1H), 9.01 (br s, 1H), 11.91 (d, 1H)

### EXAMPLE 13

### (+/−)-N-[3,5-difluoro-4-({3-[1-(trifluoromethyl)cyclobutyl]-1H-pyrrolo[2,3-b]pyridin-4-yl}oxy)phenyl]-2,7-dioxa-9-azaspiro[4.5]dec-8-en-8-amine

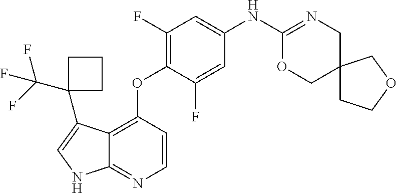 [SMILES: Fc1cc(NC2=NCC3(CCOC3)CO2)cc(F)c1Oc1ccnc2[nH]cc(C3(C(F)(F)F)CCC3)c12]

In analogy to Example 5, (+/−)-N-{3,5-difluoro-4-[(3-[1-(trifluoromethyl)cyclobutyl]-1-{[2-(trimethylsilyl)ethoxy]methyl}-1H-pyrrolo[2,3-b]pyridin-4-yl)oxy]phenyl}-2,7-dioxa-9-azaspiro[4.5]dec-8-en-8-amine (95.0 mg, 146 μmol, intermediate 48) was treated with trifluoroacetic acid (0.5 mL) in dichloromethane (1.0 mL) to afford after preparative HPLC purification the title compound (33 mg, 41% yield).

LC-MS (Method 4): Rₜ=1.20 min; MS (ESIpos): m/z=523 [M+H]⁺

¹H NMR (400 MHz, DMSO-d₆) δ ppm 1.65-1.81 (m, 2H), 1.89-2.06 (m, 2H), 2.62-2.70 (m, 4H), 3.44 (br d, 1H), 3.60 (d, 1H), 3.74-3.85 (m, 2H), 4.10 (s, 2H), 6.24 (d, 1H), 7.50 (d, 1H), 7.53 (br s, 1H), 8.05 (d, 1H), 9.07 (br s, 1H), 11.93 (br d, 1H)

### EXAMPLE 14

### N-[3,5-difluoro-4-({3-[1-(trifluoromethyl)cyclobutyl]-1H-pyrrolo[2,3-b]pyridin-4-yl}oxy)phenyl]-2,6-dioxa-8-azaspiro[3.5]non-7-en-7-amine

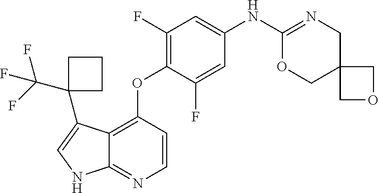 [SMILES: Fc1cc(NC2=NCC3(COC3)CO2)cc(F)c1Oc1ccnc2[nH]cc(C3(C(F)(F)F)CCC3)c12]

In analogy to Example 5, N-{3,5-difluoro-4-[(3-[1-(trifluoromethyl)cyclobutyl]-1-{[2-(trimethylsilyl)ethoxy]methyl}-1H-pyrrolo[2,3-b]pyridin-4-yl)oxy]phenyl}-2,6-dioxa-8-azaspiro[3.5]non-7-en-7-amine (175 mg, 274 μmol, intermediate 51) was treated with trifluoroacetic acid (1.0 mL) in dichloromethane (2.0 mL) to afford after preparative HPLC purification the title compound (38 mg, 26% yield).

LC-MS (Method 4): Rₜ=1.16 min; MS (ESIpos): m/z=509 [M+H]⁺

¹H NMR (400 MHz, DMSO-d₆) δ ppm 1.87-2.05 (m, 2H), 2.61-2.70 (m, 4H), 3.59 (s, 2H), 4.38-4.46 (m, 6H), 6.22 (d, 1H), 7.38 (br s, 1H), 7.50 (s, 1H), 8.04 (d, 1H), 11.94 (br s, 1H)

### Experimental Section—Biological Assays

Biological in Vitro Assays

The in vitro activity of the compounds of the present invention can be demonstrated in the following assays:

The example testing experiments described herein serve to illustrate the present invention and the invention is not limited to the examples given.

Biological Evaluation

In order that this invention may be better understood, the following examples are set forth. These examples are for the purpose of illustration only, and are not to be construed as limiting the scope of the invention in any manner. All publications mentioned herein are incorporated by reference in their entirety.

Demonstration of the activity of the compounds of the present invention may be accomplished through in vitro and in vivo assays that are well known in the art. For example, to demonstrate the efficacy of a pharmaceutical agent to inhibit and be selective against e.g. TBK1 the following assays may be used.

MAP4K1 Binding Competition Assay

The ability of the compounds of the present invention to inhibit the binding of an Alexa647-labelled ATP-competitive kinase inhibitor to a Glutathione-S-transferase-(GST-) fusion protein was quantified employing the TR-FRET-based binding competition assay as described in the following paragraphs.

A recombinant fusion protein of N-terminal GST and full-length human, expressed by baculovirus infected SF9 insect cells and purified by Glutathione Sepharose affinity chromatography, was used as GST-fusion protein. Tracer 222 from Invitrogen (catalogue no. PR9198A) was used as Alexa647-labelled ATP-competitive kinase inhibitor.

For the assay 50 nl of a 100 fold concentrated solution of the test compound in DMSO was pipetted into either a black low volume 384 well microtiter plate or a black 1536 well microtiter plate (both Greiner Bio-One, Frickenhausen, Germany), 3 μL solution of Tracer 222 (25 nM=>final concentration in 5 μL assay volume is 15 nM) in aqueous assay buffer [25 mM Tris/HCl pH 7.5, 10 mM MgCl₂, 5 mM β-glycerolphosphate, 2.5 mM dithiothreitol, 0.5 mM ethylene glycol-bis(2-aminoethylether)-N,N,N′,N′-tetraacetic acid [EGTA], 0.5 mM sodium ortho-vanadate, 0.01% (w/v) bovine serum albumin [BSA], 0.005% (w/v) Pluronic F-127 (Sigma)] were added. Then the binding competition was started by the addition of 2 μL of a solution of the GST-fusion protein (2.5 nM=>final cone, in the 5 μL assay volume is 1 nM) and of Anti-GST-Tb (1.25 nM=>final cone, in the 5 μL assay volume is 0.5 nM), a Lumi4®-Tb Cryptate-conjugated anti-GST-antibody from Cisbio Bioassays (France), in assay buffer.

The resulting mixture was incubated 30 min at 22° C. to allow the formation of a complex between the Tracer 222, the fusion protein and Anti-GST-Tb. Subsequently the amount of this complex was evaluated by measurement of the resonance energy transfer from the Tb-cryptate to the Tracer 222. Therefore, the fluorescence emissions at 620 nm and 665 nm after excitation at 350 nm were measured in a TR-FRET reader, e.g. a Pherastar (BMG Labtechnologies, Offenburg, Germany) or a Viewlux (Perkin-Elmer). The ratio of the emissions at 665 nm and at 622 nm was taken as the measure for the amount of the complex. The data were normalised (assay reaction without inhibitor=0% inhibition, all other assay components but GST-fusion protein=100% inhibition). Usually the test compounds were tested on the same microtiterplate in 11 different concentrations in the range of 20 μM to 0.07 nM (20 μM, 5.7 μM, 1.6 μM, 0.47 μM, 0.13 μM, 38 nM, 11 nM, 3.1 nM, 0.9 nM, 0.25 nM and 0.07 nM, the dilution series prepared separately before the assay on the level of the 100 fold concentrated solutions in DMSO by serial dilutions, exact concentrations may vary depending pipettors used) in duplicate values for each concentration and IC50 values were calculated using Genedata Screener™ software.

ROCK-II Kinase Assay

ROCK-II-inhibitory activity of compounds of the present invention was quantified employing the ROCK-II assay as described in the following paragraphs. In essence, the enzyme activity is measured by quantification of the adenosine-di-phosphate (ADP), which is generated as a co-product of the enzyme reaction, via the “ADP-Glo™ Kinase Assay” kit from the company Promega. This detection system works as follows: In a first step the adenosine-tri-phosphate (ATP) not consumed in the kinase reaction is quantitatively converted to cAMP employing an adenylate cyclase (“ADP-Glo-reagent”), then the adenylate cyclase is stopped and the ADP generated in the kinase reaction converted to ATP which generates in a luciferase-based reaction a glow-luminescence signal (“Kinase Detection Reagent”).

Recombinant N-terminal His6-tagged human ROCK-II (amino acids 11-552), expressed by baculovirus infected SF21 insect cells and purified via Ni²⁺-NTA-agarose affinity chromatography, was purchased from Eurofins (product no. 14-451-K) and used as enzyme. As substrate for the kinase reaction the biotinylated peptide biotin-Ahx-KEAKEKRQEQIAKRRRLSSLRASTSKSGGSQK (C-terminus in amide form) was used which can be purchased e.g. from the company Biosyntan (Berlin-Buch, Germany).

For the assay 50 nl of a 100 fold concentrated solution of the test compound in DMSO was pipetted into a white 1536 well microtiter plate (Greiner Bio-One, Frickenhausen, Germany), 2 μl of a solution of ROCK-II in aqueous assay buffer [50 mM TRIS/HCl pH 7.5, 10 mM MgCl₂, 0.1 mM EGTA, 0.001% (w/v) bovine serum albumin] were added and the mixture was incubated for 15 min at 22° C. to allow pre-binding of the test compounds to the enzyme before the start of the kinase reaction. Then the kinase reaction was started by the addition of 3 μl of a solution of ATP (16.7 μM=>final cone, in the 5 μl assay volume is 10 μM) and peptide substrate (16.7 μM=>final cone, in the pi assay volume is 10 μM) in assay buffer and the resulting mixture was incubated for a reaction time of 30 min at 22° C. The concentration of ROCK-II was adjusted depending of the activity of the enzyme lot and was chosen appropriate to have the assay in the linear range, a typical concentration is about 5 nM. The reaction was stopped by the addition of 2.5 μl of “ADP-Glo-reagent” (1:1, 5 fold diluted) and the resulting mixture was incubated at 22° C. for 1 h to convert the ATP not consumed in the kinase reaction completely to cAMP. Subsequently 2.5 μl of the “kinase detection reagent” (1.2 fold more concentrated than recommended by the producer) were added, the resulting mixture was incubated at 22° C. for 1 h and then the luminescence measured with a suitable measurement instrument (e.g. Viewlux™ from Perkin-Elmer). The amount of emitted light was taken as a measure for the amount of ADP generated and thereby for the activity of the ROCK-II.

The data were normalised (enzyme reaction without inhibitor=0% inhibition, all other assay components but no enzyme=100% inhibition). Usually the test compounds were tested on the same microtiterplate in 11 different concentrations in the range of 20 μM to 0.1 nM (20 μM, 5.7 μM, 1.6 μM, 0.47 μM, 0.13 μM, 38 nM, 11 nM, 3.1 nM, 0.9 nM, 0.25 nM and 0.07 nM, the dilution series prepared separately before the assay on the level of the 100 fold concentrated solutions in DMSO by serial dilutions, exact concentrations may vary depending pipettors used) in duplicate values for each concentration and IC₅₀ values were calculated using Genedata Screener™ software.

TABLE 1
Measured IC₅₀ values of compounds regarding MAP4K1 inhibition, ROCK-
II inhibition and the selectivity ratio between the two inhibition values.

| | MAP4K1 | ROCK-II | Ratio |
|---|---|---|---|
| Example | IC₅₀ [nM] | IC₅₀ [μM] | MAP4K1/ROCK-11 |
| 1 | 8.5 | 5.6 | 659 |
| 2 | 4.2 | 1.9 | 452 |
| 3 | 4.0 | 1.6 | 404 |
| 4 | 6.2 | 4.1 | 658 |
| 5 | 5.9 | 0.40 | 68 |
| 6 | 7.9 | 0.52 | 66 |
| 7 | 5.5 | 0.51 | 93 |
| 8 | 5.0 | 0.50 | 100 |
| 9 | 8.4 | 0.94 | 112 |
| 10 | 4.8 | 0.65 | 135 |
| 11 | 3.9 | 0.56 | 143 |
| 12 | 7.4 | 1.4 | 191 |
| 13 | 6.2 | 1.2 | 193 |
| 14 | 6.9 | 1.3 | 181 |

TBK1 High ATP Kinase Assay

TBK1-inhibitory activity of compounds of the present invention at a high ATP concentration after preincubation of enzyme and test compounds was quantified employing the TR-FRET-based TBK1 assay as described in the following paragraphs.

Recombinant full-length N-terminally His-tagged human TBK1, expressed in insect cells and purified by Ni-NTA affinity chromatography, was purchased from Life Technologies (Cat. No PR5618B) and used as enzyme. As substrate for the kinase reaction biotinylated peptide biotin-Ahx-GDEDFSSFAEPG (C-terminus in amide form) was used which can be purchased e.g. form the company Biosyntan (Berlin-Buch, Germany).

For the assay 50 nl of a 100 fold concentrated solution of the test compound in DMSO was pipetted into either a black low volume 384 well microtiter plate or a black 1536 well microtiter plate (both Greiner Bio-One, Frickenhausen, Germany), 2 μL of a solution of TBK1 in aqueous assay buffer [50 mM HEPES pH 7.0, 10 mM MgCl₂, 1.0 mM dithiothreitol, 0.05% (w/v) bovine serum albumine, 0.01% (v/v) Nonidet-P40 (Sigma), protease inhibitor mixture (“Complete w/o EDTA” from Roche, 1 tablet per 5 mL)] were added and the mixture was incubated for 15 min at 22° C. to allow pre-binding of the test compounds to the enzyme before the start of the kinase reaction. Then the kinase reaction was started by the addition of 3 μL of a solution of adenosine-tri-phosphate (ATP, 1.67 mM=>final cone, in the 5 μL assay volume is 1 mM) and substrate (1.67 μM=>final cone, in the 5 μL assay volume is 1 μM) in assay buffer and the resulting mixture was incubated for a reaction time of 30 min at 22° C. The concentration of TBK1 was adjusted depending of the activity of the enzyme lot and was chosen appropriate to have the assay in the linear range, typical concentrations were in the range of 0.002-0.004 μg/mL. The reaction was stopped by the addition of 3 μL of a solution of TR-FRET detection reagents (0.33 μM streptavidine-XL665 [Cisbio Bioassays, Codolet, France], 2.5 nM anti-phosho-Serine antibody [Merck Millipore, “STK antibody”, cat. #35-002] and 1.25 nM LANCE EU-W1024 labeled anti-mouse IgG antibody [Perkin-Elmer, product no. AD0077]) in an aqueous EDTA-solution (167 mM EDTA, 0.13% (w/v) bovine serum albumin in 100 mM HEPES/NaOH pH 7.5).

The resulting mixture was incubated 1 h at 22° C. to allow the formation of complex between the phosphorylated biotinylated peptide and the detection reagents.

Subsequently the amount of phosphorylated substrate was evaluated by measurement of the resonance energy transfer from the Eu-chelate to the streptavidine-XL. Therefore, the fluorescence emissions at 620 nm and 665 nm after excitation at 350 nm was measured in a TR-FRET reader, e.g. a Pherastar (BMG Labtechnologies, Offenburg, Germany) or a Viewlux (Perkin-Elmer). The ratio of the emissions at 665 nm and at 622 nm was taken as the measure for the amount of phosphorylated substrate. The data were normalised (enzyme reaction without inhibitor=0% inhibition, all other assay components but no enzyme=100% inhibition). Usually the test compounds were tested on the same microtiterplate in 11 different concentrations in the range of 20 μM to 0.07 nM (20 μM, 5.7 μM, 1.6 μM, 0.47 μM, 0.13 μM, 38 nM, 11 nM, 3.1 nM, 0.9 nM, 0.25 nM and 0.07 nM, the dilution series prepared separately before the assay on the level of the 100 fold concentrated solutions in DMSO by serial dilutions, exact concentrations may vary depending pipettors used) in duplicate values for each concentration and IC50 values were calculated using Genedata Screener™ software.

TABLE 2
Measured IC₅₀ values of compounds regarding
TBK1 inhibition as selectivity assay

| | TBK1 |
|---|---|
| Example | IC₅₀ [μM] |
| 1 | >20 |
| 2 | >20 |
| 3 | >20 |
| 4 | >20 |
| 5 | >20 |
| 6 | >20 |
| 7 | >20 |
| 8 | >20 |
| 9 | >20 |
| 10 | >20 |
| 11 | >20 |
| 12 | >20 |
| 13 | >20 |
| 14 | >20 |

Phosphorylation Assay in Human Cell Line

Phosphorylation assays were carried out in Jurkat E6.1 cells from American Type Culture Collection (ATCC) stably overexpressing human FLAG-tagged SLP-76 (proprietary). Cultured cells were kept in RPMI 1640 medium supplemented with 1% FCS at a cell density of 2×10e6/mL 24 h prior compound testing. Starved cells were transferred to a 384 well format plate at a cell density of 140.000 cells/well and simultaneously treated with 1 μg/mL a-CD3 antibody (clone OKT3. ebioscience #16-0037-85) and 4 μg/mL anti-IgG crosslinking antibody (Invitrogen goat anti-mouse IgG (H+L) 2 #31160) together with the test compound for 30 min at 37° C. Applied compounds were tested at either fixed concentration of 10 μmol/L and 20 μmol/L or in a 8 point dose response titration of increase compound concentration with 10 nmol/L. 50 nmol/L. 100 nmol/L. 500 nmol/L. 1 μmol/L. 5 μmol/L. 10 μmol/L and 20 μmol/L in triplicates. The cells were washed once in phosphate-buffered saline (pH 7.4). The detection of pSer376-SLP76 levels in the proprietary Jurkat cell lines was carried out utilizing an adapted protocol of the HTRF pSLP76 Assay (Cisbio #63ADK076PEG). Cells were lysed using 4 μl of the supplemented lysis buffer (Cisbio #63ADK076PEG) for 60 min at room temperature. Subsequently 4 μl of the premixed antibody solution (Cisbio #63ADK076PEG) was added and incubated over night at room temperature. Read-out and analyses was carried out using a Pherastar and the MARS software (BMG Labtechnologies, Offenburg, Germany).

As control for maximal effect (max control which represent the maximally possible inhibition of pSer376-SLP-76 by a test compound) cells with no a-CD3 (clone OKT3. ebioscience #16-0037-85) and no test compound treatment were used. Cells with a-CD3 treatment only were used as negative control (min control, which represent the minimally possible inhibition of pSer376-SLP-76 by a test compound).

TABLE 3
Measured IC₅₀ values / % amount of pSer376-SLP-76 of compound

| Example | IC₅₀ [μM] |
|---|---|
| 1 | |
| 2 | 1.3 |
| 3 | 1.2 |
| 4 | 3.0 |
| 5 | 0.94 |
| 6 | 1.3 |
| 7 | 0.89 |
| 8 | 0.33 |
| 9 | 2.7 |
| 10 | 2.0 |
| 11 | 1.1 |
| 12 | 8.5 |
| 13 | 1.5 |
| 14 | 0.51 |

Stimulation of IFNg Production from Human Primary Peripheral Blood Mononuclear Cells (PBMCs)

The effect of the compound in the activation of human T cells was tested by measuring the production of the proinflammatory cytokine IFNg in vitro. Fresh human PBMCs were isolated and activated in vitro with coated a-CD3 (clone OKT3. ebioscience #16-0037-85. plate-bound). Concentration of a-CD3 was titrated in order to obtain a sub-optimal activation of PBMCs (1×10⁶ PBMCs/mL). Cells were activated with a-CD3 and 1 μmol/L PGE2 for 22 hours in the presence of the compounds and the supernatant of the culture was isolated and tested for IFNg concentration. Applied compounds were tested at either fixed concentration of 200 nmol/L or in a 6 point dose response titration of increase compound concentration from 12 nmol/L to 3 μmol/L in triplicates. IFNg concentration was determined by ELISA (Opt EIA human IFNg ELISA BD #555142). Plate was coated with a-IFNg overnight. The plates were washed 3 times and the supernatant from the PBMCs culture was added to the wells and incubated for 2 hours. Plates were washed and detection antibody and the SAv-HRP was added for 1 h. Plates were washed and the substrate was added until the standard turns blue. The reaction is stopped by adding 50 μL 2N H2SO4. Absorbance was measured with a TECAN Reader at 450 to 570 nm. Concentration of IFNg was calculated from the absorbance using standards of known concentration.

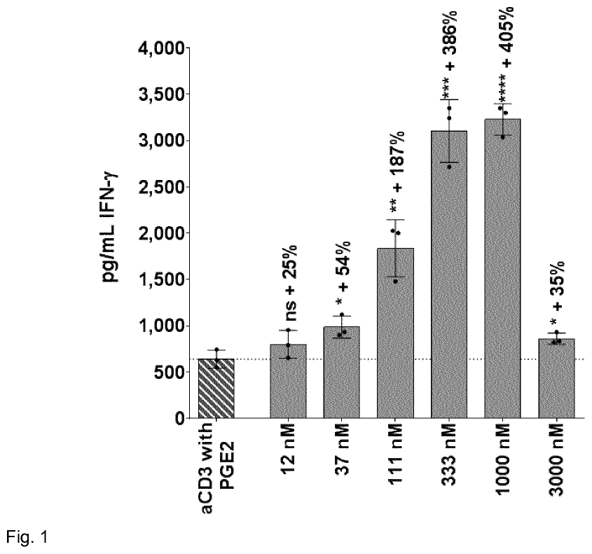

FIG. 1 shows the efficacy of a selected example in IFNγ production (Human primary peripheral blood mononuclear cells)

1. A compound of formula (I) 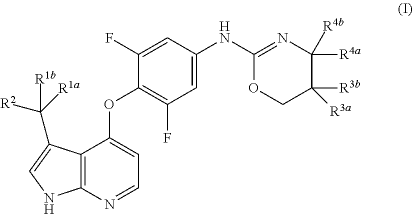
wherein
R^{1a} and R^{1b} together represent a C₃-C₆-cycloalkyl ring;
R² represents a C₁-C₃-haloalkyl;
R^{3a} and R^{3b} represent independently hydrogen or a C₁-C₃-alkyl, or
R^{3a} and R^{3b} together represent a monocyclic 3 to 6-membered cycloalkyl or 4 to 6-membered heterocycloalkyl, wherein said cycloalkyl or heterocycloalkyl is optionally substituted by fluorine, chlorine, methyl, methoxy, hydroxy, or cyano;
R^{4a} and R^{4b} represent independently hydrogen or a C₁-C₃-alkyl;
and polymorphs, enantiomers, diastereomers, racemates, tautomers, solvates, pharmaceutically acceptable salts thereof and solvates of the pharmaceutically acceptable salts.

2. The compound of general formula (I) according to claim 1, wherein
R^{1a} and R^{1b} together represent a C₃-C₅-cycloalkyl ring;
R² represents a halomethyl or haloethyl group;
R^{3a} and R^{3b} represent independently hydrogen or a methyl group, or
R^{3a} and R^{3b} together represent a monocyclic 3 to 5-membered cycloalkyl or 5 or 6-membered heterocycloalkyl, wherein said cycloalkyl or heterocycloalkyl is optionally substituted by fluorine, chlorine, methyl, methoxy, hydroxy, or cyan; and
R^{4a} and R^{4b} represent independently hydrogen or a methyl group;
and polymorphs, enantiomers, diastereomers, racemates, tautomers, solvates, pharmaceutically acceptable salts thereof, and solvates of the pharmaceutically acceptable salts.

3. The compound of general formula (I) according to claim 1, wherein
R^{1a} and R^{1b} together represent a cyclopropyl or cyclobutyl ring;
R² represents a trifluoromethyl;
R^{3a} and R^{3b} each represents a methyl group; or
R^{3a} and R^{3b} together represents a cyclopropyl or cyclobutyl ring or a 5 or 6-membered heterocycloalkyl ring having an oxygen atom as heteroatom; and
R^{4a} and R^{4b} each represents hydrogen;
and polymorphs, enantiomers, diastereomers, racemates, tautomers, solvates, pharmaceutically acceptable salts thereof, and solvates of the pharmaceutically acceptable salts.

4. The compound of general formula (I) according to claim 1, wherein
R^{1a} and R^{1b} together represent a cyclobutyl ring;
R² represents a trifluoromethyl;
R^{3a} and R^{3b} each represents a methyl group, or
R^{3a} and R^{3b} together represent a cyclopropyl or cyclobutyl ring or a 6-membered heterocycloalkyl ring having an oxygen atom as heteroatom; and
R^{4a} and R^{4b} each represents hydrogen;
and polymorphs, enantiomers, diastereomers, racemates, tautomers, solvates, pharmaceutically acceptable salts thereof, and solvates of the pharmaceutically acceptable salts.

5. The compound of general formula (I) according to claim 1, wherein
R^{1a} and R^{1b} together represent a cyclobutyl ring;
and polymorphs, enantiomers, diastereomers, racemates, tautomers, solvates, pharmaceutically acceptable salts thereof and solvates of the pharmaceutically acceptable salts.

6. The compound of general formula (I) according to claim 1, wherein
R² represents a trifluoromethyl;
and polymorphs, enantiomers, diastereomers, racemates, tautomers, solvates, pharmaceutically acceptable salts thereof and solvates of the pharmaceutically acceptable salts.

7. The compound of general formula (I) according to claim 1, wherein
R^{3a} and R^{3b} each represents a methyl group;
and polymorphs, enantiomers, diastereomers, racemates, tautomers, solvates, pharmaceutically acceptable salts thereof and solvates of the pharmaceutically acceptable salts.

8. The compound of general formula (I) according to claim 1, wherein
R^{3a} and R^{3b} together represent a cyclopropyl or cyclobutyl ring or a 6-membered heterocycloalkyl ring having an oxygen atom as hetoeroatom;
and polymorphs, enantiomers, diastereomers, racemates, tautomers, solvates, pharmaceutically acceptable salts thereof, and solvates of the pharmaceutically acceptable salts.

9. The compound of general formula (I) according to claim 1, wherein
R^{4a} and R^{4b} each represents hydrogen;
and polymorphs, enantiomers, diastereomers, racemates, tautomers, solvates, pharmaceutically acceptable salts thereof and solvates of the pharmaceutically acceptable salts.

10. The compound according to claim 1, which is selected from the group consisting of:
N-[3,5-difluoro-4-({3-[1-(trifluoromethyl)cyclobutyl]-1H-pyrrolo[2,3-b]pyridin-4-yl}oxy)phenyl]-6-oxa-8-azaspiro[3.5]non-7-en-7-amine;
N-[3,5-difluoro-4-({3-[1-(trifluoromethyl)cyclobutyl]-1H-pyrrolo[2,3-b]pyridin-4-yl}oxy)phenyl]-2,9-dioxa-4-azaspiro[5.5]undec-3-en-3-amine;
N-[3,5-difluoro-4-({3-[1-(trifluoromethyl)cyclobutyl]-1H-pyrrolo[2,3-b]pyridin-4-yl}oxy)phenyl]-5-oxa-7-azaspiro[2.5]oct-6-en-6-amine;
N-[3,5-difluoro-4-({3-[1-(trifluoromethyl)cyclobutyl]-1H-pyrrolo[2,3-b]pyridin-4-yl}oxy)phenyl]-5,5-dimethyl-5,6-dihydro-4H-1,3-oxazin-2-amine;
(+/−)-N-[3,5-difluoro-4-({3-[1-(trifluoromethyl)cyclopropyl]-1H-pyrrolo[2,3-b]pyridin-4-yl}oxy)phenyl]-2,7-dioxa-9-azaspiro[4.5]dec-8-en-8-amine;
(5R)—N-[3,5-difluoro-4-({3-[1-(trifluoromethyl)cyclopropyl]-1H-pyrrolo[2,3-b]pyridin-4-yl}oxy)phenyl]-2,7-dioxa-9-azaspiro[4.5]dec-8-en-8-amine;
(5S)—N-[3,5-difluoro-4-({3-[1-(trifluoromethyl)cyclopropyl]-1H-pyrrolo[2,3-b]pyridin-4-yl}oxy)phenyl]-2,7-dioxa-9-azaspiro[4.5]dec-8-en-8-amine;
N-[3,5-difluoro-4-({3-[1-(trifluoromethyl)cyclopropyl]-1H-pyrrolo[2,3-b]pyridin-4-yl}oxy)phenyl]-2,7-dioxa-9-azaspiro[4.5]dec-8-en-8-amine (single enantiomer 1);
N-[3,5-difluoro-4-({3-[1-(trifluoromethyl)cyclopropyl]-1H-pyrrolo[2,3-b]pyridin-4-yl}oxy)phenyl]-2,7-dioxa-9-azaspiro[4.5]dec-8-en-8-amine (single enantiomer 2);
N-[3,5-difluoro-4-({3-[1-(trifluoromethyl)cyclopropyl]-1H-pyrrolo[2,3-b]pyridin-4-yl}oxy)phenyl]-2,6-dioxa-8-azaspiro[3.5]non-7-en-7-amine;
N-[3,5-difluoro-4-({3-[1-(trifluoromethyl)cyclopropyl]-1H-pyrrolo[2,3-b]pyridin-4-yl}oxy)phenyl]-5,5-dimethyl-5,6-dihydro-4H-1,3-oxazin-2-amine;
N-[3,5-difluoro-4-({3-[1-(trifluoromethyl)cyclopropyl]-1H-pyrrolo[2,3-b]pyridin-4-yl}oxy)phenyl]-5-oxa-7-azaspiro[2.5]oct-6-en-6-amine;
N-[3,5-difluoro-4-({3-[1-(trifluoromethyl)cyclopropyl]-1H-pyrrolo[2,3-b]pyridin-4-yl}oxy)phenyl]-2,9-dioxa-4-azaspiro[5.5]undec-3-en-3-amine;
N-[3,5-difluoro-4-({3-[1-(trifluoromethyl)cyclopropyl]-1H-pyrrolo[2,3-b]pyridin-4-yl}oxy)phenyl]-6-oxa-8-azaspiro[3.5]non-7-en-7-amine;
(+/−)-N-[3,5-difluoro-4-({3-[1-(trifluoromethyl)cyclobutyl]-1H-pyrrolo[2,3-b]pyridin-4-yl}oxy)phenyl]-2,7-dioxa-9-azaspiro[4.5]dec-8-en-8-amine; and
N-[3,5-difluoro-4-({3-[1-(trifluoromethyl)cyclobutyl]-1H-pyrrolo[2,3-b]pyridin-4-yl}oxy)phenyl]-2,6-dioxa-8-azaspiro[3.5]non-7-en-7-amine.

11. (canceled)

12. (canceled)

13. A pharmaceutical composition comprising the compound of general formula (I) according to claim 1 and one or more pharmaceutically acceptable excipients.

14. A pharmaceutical combination comprising:
one or more of the compounds of general formula (I) according to claim 1, and one or more selected from the group consisting of:
one or more pharmaceutically active anti-cancer compounds, and
one or more pharmaceutically active immune checkpoint inhibitors.

15. A pharmaceutical combination according to claim 14, wherein the pharmaceutically active immune checkpoint inhibitor is an antibody.

16. A method of treating a disease, condition, or disorder comprising administering the compound of general formula (I) according to claim 1.

17. A method of preparing a pharmaceutical composition, comprising mixing the compound of general formula (I) according to claim 1 with one or more pharmaceutically acceptable excipients.

18. The method of claim 16, wherein the disease, condition, or disorder is selected from the group consisting of: cancer, a condition associated with dysregulated immune responses, a disorder associated with aberrant MAP4K1 signaling, or tumors associated with aberrant MAP4K1 signaling.

19. The method of claim 16, wherein the disease, condition, or disorder is selected from the group consisting of: benign hyperplasias, atherosclerotic disorders, sepsis, autoimmune disorders, vascular disorders, viral infections, neurodegenerative disorders, and inflammatory disorders.

20. A method of controlling male fertility comprising administering a compound of general formula (I) according to claim 1.

21. The compound of general formula (I) according to claim 2, wherein
R^{1a} and R^{1b} together represent a cyclobutyl ring;
and polymorphs, enantiomers, diastereomers, racemates, tautomers, solvates, pharmaceutically acceptable salts thereof, and solvates of the pharmaceutically acceptable salts.

22. The compound of general formula (I) according to claim 3, in which
R^{1a} and R^{1b} together represent a cyclobutyl ring;
and polymorphs, enantiomers, diastereomers, racemates, tautomers, solvates, pharmaceutically acceptable salts thereof, and solvates of the pharmaceutically acceptable salts.

23. The compound of general formula (I) according to claim 2, in which
R² represents a trifluoromethyl;
and polymorphs, enantiomers, diastereomers, racemates, tautomers, solvates, pharmaceutically acceptable salts thereof, and solvates of the pharmaceutically acceptable salts.

24. The compound of general formula (I) according to claim 2, in which
R^{3a} and R^{3b} each represents a methyl group;
and polymorphs, enantiomers, diastereomers, racemates, tautomers, solvates, pharmaceutically acceptable salts thereof, and solvates of the pharmaceutically acceptable salts.

25. The compound of general formula (I) according to claim 2, in which
R^{3a} and R^{3b} together represent a cyclopropyl or cyclobutyl ring or a 6-membered heterocycloalkyl ring having an oxygen atom as heteroatom;
and polymorphs, enantiomers, diastereomers, racemates, tautomers, solvates, pharmaceutically acceptable salts thereof, and solvates of the pharmaceutically acceptable salts.
